SEQUENCE LISTING (123 sequences)

SEQ ID NO: 1 - protein, 192 aa, Canis lupus
  misc_feature (1)..(192): Canis lupus EPO precursor form
MGACECPALFLLLSLLLLPLGLPVLGAPPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRALGAQKEAMSLPEEASPAPLRTFTVDTLCKLFRIYSNFLRGKLTLYTGEACRRGDR

SEQ ID NO: 2 - protein, 166 aa, Canis lupus
  misc_feature (1)..(166): Canis lupus EPO mature form
APPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRALGAQKEAMSLPEEASPAPLRTFTVDTLCKLFRIYSNFLRGKLTLYTGEACRRGDR

SEQ ID NO: 3 - protein, 192 aa, Equus caballus
  misc_feature (1)..(192): Equus caballus EPO precursor form
MGVRECPALLLLLSLLLPPLGLPALGAPPRLICDSRVLERYILEAREAENVTMGCAEGCSFGENVTVPDTKVNFYSWKRMEVEQQAVEVWQGLALLSEAILQGQALLANSSQPSETLRLHVDKAVSSLRSLTSLLRALGAQKEAISPPDAASAAPLRTFAVDTLCKLFRIYSNFLRGKLKLYTGEACRRGDR

SEQ ID NO: 4 - protein, 166 aa, Equus caballus
  misc_feature (1)..(166): Equus caballus EPO mature form
APPRLICDSRVLERYILEAREAENVTMGCAEGCSFGENVTVPDTKVNFYSWKRMEVEQQAVEVWQGLALLSEAILQGQALLANSSQPSETLRLHVDKAVSSLRSLTSLLRALGAQKEAISPPDAASAAPLRTFAVDTLCKLFRIYSNFLRGKLKLYTGEACRRGDR

SEQ ID NO: 5 - protein, 192 aa, Felis catus
  misc_feature (1)..(192): Felis catus EPO precursor form wild-type feline EPO G44
MGSCECPALLLLLSLLLLPLGLPVLGAPPRLICDSRVLERYILGAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 6 - protein, 166 aa, Felis catus
  misc_feature (1)..(166): Felis catus EPO mature form wild-type feline EPO G18
APPRLICDSRVLERYILGAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 7 - protein, 192 aa, Felis catus
  misc_feature (1)..(192): Felis catus EPO precursor form wild-type feline EPO E44
MGSCECPALLLLLSLLLLPLGLPVLGAPPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 8 - protein, 166 aa, Felis catus
  misc_feature (1)..(166): Felis catus EPO mature form wild-type feline EPO E18
APPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 9 - protein, 192 aa, Artificial Sequence
  Synthetic Canine EPO analog A precursor A56N G58T P113E S114N
MGACECPALFLLLSLLLLPLGLPVLGAPPRLICDSRVLERYILEAREAENVTMGCNQTCSFSENITVPDTKVNFYTWKRMDVGQQALEVWQGLALLSEAILRGQALLANASQENETPQLHVDKAVSSLRSLTSLLRALGAQKEAMSLPEEASPAPLRTFTVDTLCKLFRIYSNFLRGKLTLYTGEACRRGDR

SEQ ID NO: 10 - protein, 166 aa, Artificial Sequence
  Synthetic Canine EPO analog A mature A30N G32T P87E S88N
APPRLICDSRVLERYILEAREAENVTMGCNQTCSFSENITVPDTKVNFYTWKRMDVGQQALEVWQGLALLSEAILRGQALLANASQENETPQLHVDKAVSSLRSLTSLLRALGAQKEAMSLPEEASPAPLRTFTVDTLCKLFRIYSNFLRGKLTLYTGEACRRGDR

SEQ ID NO: 11 - protein, 192 aa, Artificial Sequence
  Synthetic Canine EPO analog B precursor A56N G58T P113V S114N
MGACECPALFLLLSLLLLPLGLPVLGAPPRLICDSRVLERYILEAREAENVTMGCNQTCSFSENITVPDTKVNFYTWKRMDVGQQALEVWQGLALLSEAILRGQALLANASQVNETPQLHVDKAVSSLRSLTSLLRALGAQKEAMSLPEEASPAPLRTFTVDTLCKLFRIYSNFLRGKLTLYTGEACRRGDR

SEQ ID NO: 12 - protein, 166 aa, Artificial Sequence
  Synthetic Canine EPO analog B mature A30N G32T P87V S88N
APPRLICDSRVLERYILEAREAENVTMGCNQTCSFSENITVPDTKVNFYTWKRMDVGQQALEVWQGLALLSEAILRGQALLANASQVNETPQLHVDKAVSSLRSLTSLLRALGAQKEAMSLPEEASPAPLRTFTVDTLCKLFRIYSNFLRGKLTLYTGEACRRGDR

SEQ ID NO: 13 - protein, 192 aa, Artificial Sequence
  Synthetic Canine EPO analog C precursor D81N G83T
MGACECPALFLLLSLLLLPLGLPVLGAPPRLICDSRVLERYILEAREAENVTMGCAQGCSFSENITVPDTKVNFYTWKRMNVTQQALEVWQGLALLSEAILRGQALLANASQPSETPQLHVDKAVSSLRSLTSLLRALGAQKEAMSLPEEASPAPLRTFTVDTLCKLFRIYSNFLRGKLTLYTGEACRRGDR

SEQ ID NO: 14 - protein, 166 aa, Artificial Sequence
  Synthetic Canine EPO analog C mature D55N G57T
APPRLICDSRVLERYILEAREAENVTMGCAQGCSFSENITVPDTKVNFYTWKRMNVTQQALEVWQGLALLSEAILRGQALLANASQPSETPQLHVDKAVSSLRSLTSLLRALGAQKEAMSLPEEASPAPLRTFTVDTLCKLFRIYSNFLRGKLTLYTGEACRRGDR

SEQ ID NO: 15 - protein, 192 aa, Artificial Sequence
  Synthetic Canine EPO analog D precursor L138N A140T
MGACECPALFLLLSLLLLPLGLPVLGAPPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRANGTQKEAMSLPEEASPAPLRTFTVDTLCKLFRIYSNFLRGKLTLYTGEACRRGDR

SEQ ID NO: 16 - protein, 166 aa, Artificial Sequence
  Synthetic Canine EPO analog D mature L112N A114T
APPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRANGTQKEAMSLPEEASPAPLRTFTVDTLCKLFRIYSNFLRGKLTLYTGEACRRGDR

SEQ ID NO: 17 - protein, 192 aa, Artificial Sequence
  Synthetic Canine EPO analog E precursor L147N P148V E149T
MGACECPALFLLLSLLLLPLGLPVLGAPPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRALGAQKEAMSNVTEASPAPLRTFTVDTLCKLFRIYSNFLRGKLTLYTGEACRRGDR

SEQ ID NO: 18 - protein, 166 aa, Artificial Sequence
  Synthetic Canine EPO analog E mature L121N P122V E123T
APPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRALGAQKEAMSNVTEASPAPLRTFTVDTLCKLFRIYSNFLRGKLTLYTGEACRRGDR

SEQ ID NO: 19 - protein, 192 aa, Artificial Sequence
  Synthetic Canine EPO analog F precursor P148E E149N A151T
MGACECPALFLLLSLLLLPLGLPVLGAPPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRALGAQKEAMSLENETSPAPLRTFTVDTLCKLFRIYSNFLRGKLTLYTGEACRRGDR

SEQ ID NO: 20 - protein, 166 aa, Artificial Sequence
  Synthetic Canine EPO analog F mature P122E E123N A125T
APPRLICDSRVLERYILEAREAENVTMGCAQGCSFSENITVPDTKVNFYTWKRMDVGQQALEVWQGLALLSEAILRGQALLANASQPSETPQLHVDKAVSSLRSLTSLLRALGAQKEAMSLENETSPAPLRTFTVDTLCKLFRIYSNFLRGKLTLYTGEACRRGDR

SEQ ID NO: 21 - protein, 192 aa, Artificial Sequence
  Synthetic Canine EPO analog G precursor A45C S172C
MGACECPALFLLLSLLLLPLGLPVLGAPPRLICDSRVLERYILECREAENVTMGCAQGCSFSENITVPDTKVNFYTWKRMDVGQQALEVWQGLALLSEAILRGQALLANASQPSETPQLHVDKAVSSLRSLTSLLRALGAQKEAMSLPEEASPAPLRTFTVDTLCKLFRIYCNFLRGKLTLYTGEACRRGDR

SEQ ID NO: 22 - protein, 166 aa, Artificial Sequence
  Synthetic Canine EPO analog G mature A19C S146C
APPRLICDSRVLERYILECREAENVTMGCAQGCSFSENITVPDTKVNFYTWKRMDVGQQALEVWQGLALLSEAILRGQALLANASQPSETPQLHVDKAVSSLRSLTSLLRALGAQKEAMSLPEEASPAPLRTFTVDTLCKLFRIYCNFLRGKLTLYTGEACRRGDR

SEQ ID NO: 23 - protein, 192 aa, Artificial Sequence
  Synthetic Canine EPO analog H precursor A48C H120C
MGACECPALFLLLSLLLLPLGLPVLGAPPRLICDSRVLERYILEARECENVTMGCAQGCSFSENITVPDTKVNFYTWKRMDVGQQALEVWQGLALLSEAILRGQALLANASQPSETPQLCVDKAVSSLRSLTSLLRALGAQKEAMSLPEEASPAPLRTFTVDTLCKLFRIYSNFLRGKLTLYTGEACRRGDR

SEQ ID NO: 24 - protein, 166 aa, Artificial Sequence
  Synthetic Canine EPO analog H mature A22C H94C
APPRLICDSRVLERYILEARECENVTMGCAQGCSFSENITVPDTKVNFYTWKRMDVGQQALEVWQGLALLSEAILRGQALLANASQPSETPQLCVDKAVSSLRSLTSLLRALGAQKEAMSLPEEASPAPLRTFTVDTLCKLFRIYSNFLRGKLTLYTGEACRRGDR

SEQ ID NO: 25 - protein, 192 aa, Artificial Sequence
  Synthetic Canine EPO analog I precursor E49C S172C
MGACECPALFLLLSLLLLPLGLPVLGAPPRLICDSRVLERYILEAREACNVTMGCAQGCSFSENITVPDTKVNFYTWKRMDVGQQALEVWQGLALLSEAILRGQALLANASQPSETPQLHVDKAVSSLRSLTSLLRALGAQKEAMSLPEEASPAPLRTFTVDTLCKLFRIYCNFLRGKLTLYTGEACRRGDR

SEQ ID NO: 26 - protein, 166 aa, Artificial Sequence
  Synthetic Canine EPO analog I mature E23C S146C
APPRLICDSRVLERYILEAREACNVTMGCAQGCSFSENITVPDTKVNFYTWKRMDVGQQALEVWQGLALLSEAILRGQALLANASQPSETPQLHVDKAVSSLRSLTSLLRALGAQKEAMSLPEEASPAPLRTFTVDTLCKLFRIYCNFLRGKLTLYTGEACRRGDR

SEQ ID NO: 27 - protein, 192 aa, Artificial Sequence
  Synthetic Canine EPO analog J precursor P68C G92C
MGACECPALFLLLSLLLLPLGLPVLGAPPRLICDSRVLERYILEAREAENVTMGCAQGCSFSENITVCDTKVNFYTWKRMDVGQQALEVWQCLALLSEAILRGQALLANASQPSETPQLHVDKAVSSLRSLTSLLRALGAQKEAMSLPEEASPAPLRTFTVDTLCKLFRIYSNFLRGKLTLYTGEACRRGDR

SEQ ID NO: 28 - protein, 166 aa, Artificial Sequence
  Synthetic Canine EPO analog J mature P42C G66C
APPRLICDSRVLERYILEAREAENVTMGCAQGCSFSENITVCDTKVNFYTWKRMDVGQQALEVWQCLALLSEAILRGQALLANASQPSETPQLHVDKAVSSLRSLTSLLRALGAQKEAMSLPEEASPAPLRTFTVDTLCKLFRIYSNFLRGKLTLYTGEACRRGDR

SEQ ID NO: 29 - protein, 192 aa, Artificial Sequence
  Synthetic Canine EPO analog K precursor W90C A144C
MGACECPALFLLLSLLLLPLGLPVLGAPPRLICDSRVLERYILEAREAENVTMGCAQGCSFSENITVPDTKVNFYTWKRMDVGQQALEVCQGLALLSEAILRGQALLANASQPSETPQLHVDKAVSSLRSLTSLLRALGAQKECMSLPEEASPAPLRTFTVDTLCKLFRIYSNFLRGKLTLYTGEACRRGDR

SEQ ID NO: 30 - protein, 166 aa, Artificial Sequence
  Synthetic Canine EPO analog K mature W64C A118C
APPRLICDSRVLERYILEAREAENVTMGCAQGCSFSENITVPDTKVNFYTWKRMDVGQQALEVCQGLALLSEAILRGQALLANASQPSETPQLHVDKAVSSLRSLTSLLRALGAQKECMSLPEEASPAPLRTFTVDTLCKLFRIYSNFLRGKLTLYTGEACRRGDR

SEQ ID NO: 31 - protein, 192 aa, Artificial Sequence
  Synthetic Canine EPO analog L precursor A86C E143C
MGACECPALFLLLSLLLLPLGLPVLGAPPRLICDSRVLERYILEAREAENVTMGCAQGCSFSENITVPDTKVNFYTWKRMDVGQQCLEVWQGLALLSEAILRGQALLANASQPSETPQLHVDKAVSSLRSLTSLLRALGAQKCAMSLPEEASPAPLRTFTVDTLCKLFRIYSNFLRGKLTLYTGEACRRGDR

SEQ ID NO: 32 - protein, 166 aa, Artificial Sequence
  Synthetic Canine EPO analog L mature A60C E117C
APPRLICDSRVLERYILEAREAENVTMGCAQGCSFSENITVPDTKVNFYTWKRMDVGQQCLEVWQGLALLSEAILRGQALLANASQPSETPQLHVDKAVSSLRSLTSLLRALGAQKCAMSLPEEASPAPLRTFTVDTLCKLFRIYSNFLRGKLTLYTGEACRRGDR

SEQ ID NO: 33 - protein, 508 aa, Canis lupus familiaris
  misc_feature (1)..(508): Canis lupus EPO receptor precursor form
MNHLWTHLWPGVGSLCLLLAGAAWASLPKPLDPKFESKAALLAARAPEELLCFTERLEDLVCFWEEAASAGVGPDNYSFFYQLEGEPWKTCSLHQAPTTRGAVRFWCSLPTADTSSFVPLELRATAVSSGALLYRRIIHINEVVLLDPPAGLLARRADEGGHVVLRWLPPPGAPVASLIRYEVNISGSVAGGSQKVEILDGRTECVLSNLRGGTRYTFMVRARMAEPSFGGFWSAWSEPASLLTASDLDPLILTLSLILVLILLLLAVLALLSHRRTLKQKIWPGIPSPESEFEGLFTTHKGNFQLWLYQNEGCLWWSPCTPLAEDPPAPLEVLSERCWGAPQAVEPGADDEGPLLEPVGSEHSQDTYLVLDKWLLPRNPSSEDVSQSGGSLDIVAMDKGSEASSCSSGLSLKPGPEGALGASFEYTILDPSSQLLCPRALPPELPPTPPHIKYLYLMVSDSGISTDYSSGGSQGAQGDSLNSPFLNPYENSLIPAPEPSPPGYVACS

SEQ ID NO: 34 - protein, 249 aa, Canis lupus familiaris
  misc_feature (1)..(249): Exemplary canine EPOR ECD
MNHLWTHLWPGVGSLCLLLAGAAWASLPKPLDPKFESKAALLAARAPEELLCFTERLEDLVCFWEEAASAGVGPDNYSFFYQLEGEPWKTCSLHQAPTTRGAVRFWCSLPTADTSSFVPLELRATAVSSGALLYRRIIHINEVVLLDPPAGLLARRADEGGHVVLRWLPPPGAPVASLIRYEVNISGSVAGGSQKVEILDGRTECVLSNLRGGTRYTFMVRARMAEPSFGGFWSAWSEPASLLTASDLD

SEQ ID NO: 35 - protein, 213 aa, Canis lupus familiaris
  misc_feature (1)..(213): Exemplary canine EPOR minimal ECD
DPKFESKAALLAARAPEELLCFTERLEDLVCFWEEAASAGVGPDNYSFFYQLEGEPWKTCSLHQAPTTRGAVRFWCSLPTADTSSFVPLELRATAVSSGALLYRRIIHINEVVLLDPPAGLLARRADEGGHVVLRWLPPPGAPVASLIRYEVNISGSVAGGSQKVEILDGRTECVLSNLRGGTRYTFMVRARMAEPSFGGFWSAWSEPASLLT

SEQ ID NO: 36 - protein, 486 aa, Artificial Sequence
  Synthetic Exemplary canine EPOR ECD  Human Fc
MNHLWTHLWPGVGSLCLLLAGAAWASLPKPLDPKFESKAALLAARAPEELLCFTERLEDLVCFWEEAASAGVGPDNYSFFYQLEGEPWKTCSLHQAPTTRGAVRFWCSLPTADTSSFVPLELRATAVSSGALLYRRIIHINEVVLLDPPAGLLARRADEGGHVVLRWLPPPGAPVASLIRYEVNISGSVAGGSQKVEILDGRTECVLSNLRGGTRYTFMVRARMAEPSFGGFWSAWSEPASLLTASDLDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 509 aa, Equus caballus
  misc_feature (1)..(509): Equus caballus EPO receptor precursor form
MNHLGAPLWPGVGSLCLLLAGAAWAPPPNSSDPRFESKAALLAARGPEELLCFTERLEDLVCFWEEAASAGVGPENYSFSYQLEGEPWKPCRLHQASTARGAVRFWCSLPTADTSSFVPLELRVTAATSGAPRYRRVIQVNEVVLLDPPAGLLARLADEGGHVLLRWLPPPGAPMASLIRYEVNISAGNAAGGAQRVEILDGRTECVLSNLRGQTRYTFAVRARMAEPSFGGFWSAWSEPASLLTASDLDPLLLTLSLILVLILLLLAVLALLSHRRALKQKIWPGIPSPESEFEGLFTTHKGNFQLWLYQNDGCLWWNPCTPFTEDPPASLEVLSERCWGVTQAVEPGAEDEGPLLEPVGSEHARDPYLVLDKWLLPRSPTSEDLPQPGGGLDTAAMDAGSEASSCSSALALKPGPEGASAASFEYTILDPSSQLLRPRALPPELPPTPPHLKYLYLVVSDSGISTDYSSGGSQGAQRGSSDGPYSNPYENSLVPAPEPSAPSYVACS

SEQ ID NO: 38 - protein, 250 aa, Equus caballus
  misc_feature (1)..(250): Exemplary equine EPOR ECD
MNHLGAPLWPGVGSLCLLLAGAAWAPPPNSSDPRFESKAALLAARGPEELLCFTERLEDLVCFWEEAASAGVGPENYSFSYQLEGEPWKPCRLHQASTARGAVRFWCSLPTADTSSFVPLELRVTAATSGAPRYRRVIQVNEVVLLDPPAGLLARLADEGGHVLLRWLPPPGAPMASLIRYEVNISAGNAAGGAQRVEILDGRTECVLSNLRGQTRYTFAVRARMAEPSFGGFWSAWSEPASLLTASDLD

SEQ ID NO: 39 - protein, 214 aa, Equus caballus
  misc_feature (1)..(214): Exemplary equine EPOR minimal ECD
DPRFESKAALLAARGPEELLCFTERLEDLVCFWEEAASAGVGPENYSFSYQLEGEPWKPCRLHQASTARGAVRFWCSLPTADTSSFVPLELRVTAATSGAPRYRRVIQVNEVVLLDPPAGLLARLADEGGHVLLRWLPPPGAPMASLIRYEVNISAGNAAGGAQRVEILDGRTECVLSNLRGQTRYTFAVRARMAEPSFGGFWSAWSEPASLLT

SEQ ID NO: 40 - protein, 487 aa, Artificial Sequence
  Synthetic Exemplary equine EPOR ECD  Human Fc
MNHLGAPLWPGVGSLCLLLAGAAWAPPPNSSDPRFESKAALLAARGPEELLCFTERLEDLVCFWEEAASAGVGPENYSFSYQLEGEPWKPCRLHQASTARGAVRFWCSLPTADTSSFVPLELRVTAATSGAPRYRRVIQVNEVVLLDPPAGLLARLADEGGHVLLRWLPPPGAPMASLIRYEVNISAGNAAGGAQRVEILDGRTECVLSNLRGQTRYTFAVRARMAEPSFGGFWSAWSEPASLLTASDLDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 512 aa, Felis catus
  misc_feature (1)..(512): Felis catus  EPO receptor Sequence EPOR201
MDHLWAPLWPGVGSLCLLLAGAAWAPPPNPLDPKFESKVNMVCMRAPEASACGSSERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLDPLILTLSLILVLILLLLAVLALLSHRRFTRTLKQKIWPGIPSPESEFEGLFTTHKGNFQLWLYQNEGCLWWSPCAPFAEDPPSPLEVLSERCWGATQAAEPGAEEGPLLEPLGSEHTQDTYLVLDKWLLPRNPPSEDLPRPDGSLDMVAMHKGSEASSCSSALSLKPGPEGALGASFEYTILDPSSQLLRPRALPPELPPTPPHIKYLYLMVSDSGISTDYSSGGSQEAQGDSSTGPYLNPYENSLIPATETSPPSYVACS

SEQ ID NO: 42 - protein, 226 aa, Felis catus
  misc_feature (1)..(226): Exemplary Feline EPOR201 ECD
PPPNPLDPKFESKVNMVCMRAPEASACGSSERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLD

SEQ ID NO: 43 - protein, 215 aa, Felis catus
  misc_feature (1)..(215): Exemplary Feline EPOR201 minimal ECD
DPKFESKVNMVCMRAPEASACGSSERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLT

SEQ ID NO: 44 - protein, 511 aa, Felis catus
  misc_feature (1)..(511): Felis catus  EPO receptor Sequence EPOR202
MDHLWAPLWPGVGSLCLLLAGAAWAPPPNPLDPKFESKGKDGSVCRPPQWFLEGNAEERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLDPLILTLSLILVLILLLLAVLALLSHRRTLKQKIWPGIPSPESEFEGLFTTHKGNFQLWLYQNEGCLWWSPCAPFAEDPPSPLEVLSERCWGATQAAEPGAEEGPLLEPLGSEHTQDTYLVLDKWLLPRNPPSEDLPRPDGSLDMVAMHKGSEASSCSSALSLKPGPEGALGASFEYTILDPSSQLLRPRALPPELPPTPPHIKYLYLMVSDSGISTDYSSGGSQEAQGDSSTGPYLNPYENSLIPATETSPPSYVACS

SEQ ID NO: 45 - protein, 228 aa, Felis catus
  misc_feature (1)..(228): Exemplary feline EPOR202 ECD
PPPNPLDPKFESKGKDGSVCRPPQWFLEGNAEERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLD

SEQ ID NO: 46 - protein, 217 aa, Felis catus
  misc_feature (1)..(217): Exemplary feline EPOR202 minimal ECD
DPKFESKGKDGSVCRPPQWFLEGNAEERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLT

SEQ ID NO: 47 - protein, 508 aa, Felis catus
  misc_feature (1)..(508): Felis catus  EPO receptor Sequence EPOR203
  misc_feature (39)..(39): Xaa can be any naturally occurring amino acid
MDHLWAPLWPGVGSLCLLLAGAAWAPPPNPLDPKFESKXALLAARGPEELLCFTERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLDPLILTLSLILVLILLLLAVLALLSHRRTLKQKIWPGIPSPESEFEGLFTTHKGNFQLWLYQNEGCLWWSPCAPFAEDPPSPLEVLSERCWGATQAAEPGAEEGPLLEPLGSEHTQDTYLVLDKWLLPRNPPSEDLPRPDGSLDMVAMHKGSEASSCSSALSLKPGPEGALGASFEYTILDPSSQLLRPRALPPELPPTPPHIKYLYLMVSDSGISTDYSSGGSQEAQGDSSTGPYLNPYENSLIPATETSPPSYVACS

SEQ ID NO: 48 - protein, 226 aa, Felis catus
  misc_feature (1)..(226): Exemplary feline EPOR203 ECD
  misc_feature (14)..(14): Xaa can be any naturally occurring amino acid
PPPNPLDPKFESKXALLAARGPEELLCFTERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLDP

SEQ ID NO: 49 - protein, 214 aa, Felis catus
  misc_feature (1)..(214): Exemplary feline EPOR203_39A minimal ECD
  misc_feature (8)..(8): Xaa can be any naturally occurring amino acid
DPKFESKXALLAARGPEELLCFTERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLT

SEQ ID NO: 50 - protein, 508 aa, Felis catus
  misc_feature (1)..(508): Exemplary feline EPO receptor EPOR203_39A
MDHLWAPLWPGVGSLCLLLAGAAWAPPPNPLDPKFESKAALLAARGPEELLCFTERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLDPLILTLSLILVLILLLLAVLALLSHRRTLKQKIWPGIPSPESEFEGLFTTHKGNFQLWLYQNEGCLWWSPCAPFAEDPPSPLEVLSERCWGATQAAEPGAEEGPLLEPLGSEHTQDTYLVLDKWLLPRNPPSEDLPRPDGSLDMVAMHKGSEASSCSSALSLKPGPEGALGASFEYTILDPSSQLLRPRALPPELPPTPPHIKYLYLMVSDSGISTDYSSGGSQEAQGDSSTGPYLNPYENSLIPATETSPPSYVACS

SEQ ID NO: 51 - protein, 226 aa, Felis catus
  misc_feature (1)..(226): Exemplary feline EPOR203_39A ECD
PPPNPLDPKFESKAALLAARGPEELLCFTERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLTASDLDP

SEQ ID NO: 52 - protein, 214 aa, Felis catus
  misc_feature (1)..(214): Exemplary feline EPOR203_39A minimal ECD
DPKFESKAALLAARGPEELLCFTERLEDLVCFWEEAASAGVGPDNYSFFYQLEGEPWKPCSLHQAPTARGAVRFWCSLPTADASSFVPLELRVTAVSSGAPRYHRIIHINEVVLLDPPAGLLARRADEGGHVVLRWLPPPGAPVASLIRYEVNISSGNVAGGAQKVEILDGRTECALSNLRGRTRYTFMVRARMAEPSFGGFWSAWSEPASLLT

SEQ ID NO: 53 - protein, 221 aa, Artificial Sequence
  Synthetic Exemplary wild-type canine IgG-A Fc Protein A  C1q CD16
PVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 54 - protein, 220 aa, Artificial Sequence
  Synthetic Exemplary wild-type canine IgG-B Fc Protein A + C1q + CD16 +
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 55 - protein, 237 aa, Artificial Sequence
  Synthetic Exemplary wild-type canine IgG-B Fc with hinge Protein A + C1q + CD16 +
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 56 - protein, 220 aa, Artificial Sequence
  Synthetic Exemplary wild-type canine IgG-C Fc Protein A C1q + CD16 +
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 57 - protein, 235 aa, Artificial Sequence
  Synthetic Exemplary wild-type canine IgG-C Fc with hinge Protein A C1q + CD16 +
AKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 58 - protein, 221 aa, Artificial Sequence
  Synthetic Exemplary wild-type canine IgG-D Fc Protein A C1q CD16
PVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 59 - protein, 221 aa, Artificial Sequence
  Synthetic Exemplary variant canine IgG-A Fc C1q Protein A + I(21)T R(23)L T(25)A E(80)G T(205)A Q(207)H
PVPEPLGGPSVLIFPPKPKDTLLIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 60 - protein, 220 aa, Artificial Sequence
  Synthetic Exemplary variant canine IgG-C Fc C1q + Protein A + I(21)T V(23)L T(24)I
PGCGLLGGPSVFIFPPKPKDTLLIARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 61 - protein, 221 aa, Artificial Sequence
  Synthetic Exemplary variant canine IgG-D Fc C1q Protein A + I(21)T R(23)L T(25)A E(80)G Q(207)H
PVPESLGGPSVFIFPPKPKDTLLIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 62 - protein, 221 aa, Artificial Sequence
  Synthetic Exemplary variant canine IgG-A Fc C1q  Protein A + I(21)T Q(207)H
PVPEPLGGPSVLIFPPKPKDTLRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLHNHYTDLSLSHSPGK

SEQ ID NO: 63 - protein, 220 aa, Artificial Sequence
  Synthetic Exemplary variant canine IgG-C Fc C1q + Protein A + I(21)T
PGCGLLGGPSVFIFPPKPKDTLVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 64 - protein, 221 aa, Artificial Sequence
  Synthetic Exemplary variant canine IgG-D Fc C1q  Protein A + I(21)T Q(207)H
PVPESLGGPSVFIFPPKPKDTLRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 65 - protein, 220 aa, Artificial Sequence
  Synthetic Exemplary variant canine IgG-B Fc Protein A + C1q K(93)R
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 66 - protein, 220 aa, Artificial Sequence
  Synthetic Exemplary variant canine IgG-C Fc Protein A  C1q CD16 + K(93)R
PGCGLLGGPSVFIFPPKPKDILVTARTPTVTCVVVDLDPENPEVQISWFVDSKQVQTANTQPREEQSNGTYRVVSVLPIGHQDWLSGKQFKCRVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 67 - protein, 220 aa, Artificial Sequence
  Synthetic Exemplary variant canine IgG-B Fc Protein A + C1q + CD16   M(5)P
PAPEPLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 68 - protein, 220 aa, Artificial Sequence
  Synthetic Exemplary variant canine IgG-B Fc Protein A + C1q + CD16   P(39)R
PAPEMLGGPSVFIFPPKPKDTLLIARTPEVTCVVVDLDREDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 69 - protein, 220 aa, Artificial Sequence
  Synthetic Exemplary variant canine IgG-B Fc Protein A + C1q + CD16 D(38)G
PAPEMLGGPSVFIFPPKPKDTLLIARTPEVTCVVVDLGPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 70 - protein, 220 aa, Artificial Sequence
  Synthetic Exemplary variant canine IgG-B Fc Protein A + C1q + CD16 D(38)G P(39)R
PAPEMLGGPSVFIFPPKPKDTLLIARTPEVTCVVVDLGREDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 71 - protein, 220 aa, Artificial Sequence
  Synthetic Exemplary variant canine IgG-B Fc Protein A + C1q + CD16 K(97)I
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNIALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 72 - protein, 220 aa, Artificial Sequence
  Synthetic Exemplary variant canine IgG-B Fc Protein A + C1q + CD16 A(98)G
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 73 - protein, 220 aa, Artificial Sequence
  Synthetic Exemplary variant canine IgG-B Fc Protein A + C1q + CD16  D(38)G  K(97)I A(98)G
PAPEMLGGPSVFIFPPKPKDTLLIARTPEVTCVVVDLGPEDPEVQISWFVDGKQMQTAKTQPREEQFNGTYRVVSVLPIGHQDWLKGKQFTCKVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 74 - protein, 220 aa, Artificial Sequence
  Synthetic Exemplary variant canine IgG-B Fc Protein A + C1q + CD16 M(5)P P(39)R
PAPEPLGGPSVFIFPPKPKDTLLIARTPEVTCVVVDLDREDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 75 - protein, 220 aa, Artificial Sequence
  Synthetic Exemplary variant canine IgG-C Fc Protein A  C1q + CD16 L(5)P
PGCGPLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 76 - protein, 220 aa, Artificial Sequence
  Synthetic Exemplary variant canine IgG-C Fc Protein A  C1q + CD16 P(39)R
PGCGLLGGPSVFIFPPKPKDILVTARTPTVTCVVVDLDRENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 77 - protein, 220 aa, Artificial Sequence
  Synthetic Exemplary variant canine IgG-C Fc Protein A  C1q + CD16 D(38)G
PGCGLLGGPSVFIFPPKPKDILVTARTPTVTCVVVDLGPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 78 - protein, 220 aa, Artificial Sequence
  Synthetic Exemplary variant canine IgG-C Fc Protein A  C1q + CD16 K(97)I
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNIALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 79 - protein, 220 aa, Artificial Sequence
  Synthetic Exemplary variant canine IgG-C Fc Protein A  C1q + CD16 A(98)G
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKGLPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 80 - protein, 220 aa, Artificial Sequence
  Synthetic Exemplary variant canine IgG-C Fc Protein A C1q + CD16 L(5)P P(39)R
PGCGPLGGPSVFIFPPKPKDILVTARTPTVTCVVVDLDRENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 81 - protein, 220 aa, Artificial Sequence
  Synthetic Exemplary variant canine IgG-C Fc Protein A  C1q + CD16 D(38)G K(97)I A(98)G
PGCGLLGGPSVFIFPPKPKDILVTARTPTVTCVVVDLGPENPEVQISWFVDSKQVQTANTQPREEQSNGTYRVVSVLPIGHQDWLSGKQFKCKVNNIGLPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 82 - protein, 220 aa, Artificial Sequence
  Synthetic Exemplary variant canine IgG-C Fc C1q K(93)R Protein A + I(21)T V(23)L T(24)I
PGCGLLGGPSVFIFPPKPKDTLLIARTPTVTCVVVDLDPENPEVQISWFVDSKQVQTANTQPREEQSNGTYRVVSVLPIGHQDWLSGKQFKCRVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 83 - protein, 220 aa, Artificial Sequence
  Synthetic Exemplary variant canine IgG-B Fc Protein A + C1q CD16  D(38)G  K(93)R K(97)I A(98)G
PAPEMLGGPSVFIFPPKPKDTLLIARTPEVTCVVVDLGPEDPEVQISWFVDGKQMQTAKTQPREEQFNGTYRVVSVLPIGHQDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 84 - protein, 220 aa, Artificial Sequence
  Synthetic Exemplary variant canine IgG-B Fc Protein A + C1q CD16  M(5)P P(39)R K(93)R
PAPEPLGGPSVFIFPPKPKDTLLIARTPEVTCVVVDLDREDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 85 - protein, 220 aa, Artificial Sequence
  Synthetic Exemplary variant canine IgG-C Fc Protein A  C1q CD16 D(38)G K(93)R K(97)I A(98)G
PGCGLLGGPSVFIFPPKPKDILVTARTPTVTCVVVDLGPENPEVQISWFVDSKQVQTANTQPREEQSNGTYRVVSVLPIGHQDWLSGKQFKCRVNNIGLPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 86 - protein, 220 aa, Artificial Sequence
  Synthetic Exemplary variant canine IgG-C Fc Protein A C1q CD16 M(5)P P(39)R K(93)R
PGCGPLGGPSVFIFPPKPKDILVTARTPTVTCVVVDLDRENPEVQISWFVDSKQVQTANTQPREEQSNGTYRVVSVLPIGHQDWLSGKQFKCRVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 87 - protein, 214 aa, Artificial Sequence
  Synthetic Exemplary wild-type equine IgG1 Fc Protein A + C1q +
GGPSVFLFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDESKKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 88 - protein, 214 aa, Artificial Sequence
  Synthetic Exemplary wild-type equine IgG2 Fc Protein A C1q
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 89 - protein, 244 aa, Artificial Sequence
  Synthetic Exemplary wild-type equine IgG2 Fc with hinge Protein A C1q
PPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 90 - protein, 214 aa, Artificial Sequence
  Synthetic Exemplary wild-type equine IgG3 Fc Protein A + C1q+
GGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFYPPDITVEWQSNEHPEPEGKYRTTEAQKDSDGSYFLYSKLTVEKDRWQQGTTFTCVVMHEALHNHVMQKNISKNPGK

SEQ ID NO: 91 - protein, 214 aa, Artificial Sequence
  Synthetic Exemplary wild-type equine IgG4 Fc Protein A + C1q +
VGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISAPTGQPREPQVYVLAPHRDELSKNKVSVTCLVKDFYPPDIDIEWKSNGQPEPETKYSTTPAQLDSDGSYFLYSKLTVETNRWQQGTTFTCAVMHEALHNHYTEKSVSKSPGK

SEQ ID NO: 92 - protein, 214 aa, Artificial Sequence
  Synthetic Exemplary wild-type equine IgG5 Fc Protein A  C1q
GGPSVFIFPPKPKDVLMISRKPEVTCVVVDLGHDDPDVQFTWFVDGVETHTATTEPKEEQFNSTYRVVSVLPIQHQDWLSGKEFKCSVTSKALPAPVERTISKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 93 - protein, 214 aa, Artificial Sequence
  Synthetic Exemplary wild-type equine IgG6 Fc Protein A  C1q
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPQVYILAPHPDEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 94 - protein, 214 aa, Artificial Sequence
  Synthetic Exemplary wild-type equine IgG7 Fc Protein A + C1q +
VGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHPDELSKNKVSVTCLVKDFYPPDIDIEWKSNGQPEPETKYSTTPAQLDGDGSYFLYSKLTVETNRWQQGTTFTCAVMHEALHNHYTEKSVSKSPGK

SEQ ID NO: 95 - protein, 214 aa, Artificial Sequence
  Synthetic Exemplary variant equine IgG2 Fc C1q Protein A + F(203)Y
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQGESFTCAVMHEALHNHYTKTDISESLGK

SEQ ID NO: 96 - protein, 214 aa, Artificial Sequence
  Synthetic Exemplary variant equine IgG2 Fc C1q  Protein A + A(15)T F(203)Y
GGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 97 - protein, 214 aa, Artificial Sequence
  Synthetic Exemplary variant equine IgG5 Fc C1q Protein A + V(199)L E(200)H
GGPSVFIFPPKPKDVLMISRKPEVTCVVVDLGHDDPDVQFTWFVDGVETHTATTEPKEEQFNSTYRVVSVLPIQHQDWLSGKEFKCSVTSKALPAPVERTISKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEALHNHYTQKNVSHSPGK

SEQ ID NO: 98 - protein, 214 aa, Artificial Sequence
  Synthetic Exemplary variant equine IgG6 Fc C1q  Protein A + I(199)L R(200)H H(201)N T(202)H
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPQVYILAPHPDEVTKNTVSVTCLVKDFYPPDINVEWQSNEEPEPEVKYSTTPAQLDGDGSYFLYSKLTVETDRWEQGESFTCVVMHEALHNHYRQKSITNFPGK

SEQ ID NO: 99 - protein, 214 aa, Artificial Sequence
  Synthetic Exemplary variant equine IgG1 Fc Protein A + C1q K(87)S
GGPSVFLFPPNPKDTLMITRTPEVTCVVVDVSQENPDVKFNWYMDGVEVRTATTRPKEEQFNSTYRVVSVLRIQHQDWLSGKEFKCSVNNQALPQPIERTITKTKGRSQEPQVYVLAPHPDESKKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 100 - protein, 214 aa, Artificial Sequence
  Synthetic Exemplary variant equine IgG3 Fc Protein A + C1q K(87)S
GGPSVFIFPPKPKDVLMITRMPEVTCLVVDVSHDSSDVLFTWYVDGTEVKTAKTMPNEEQNNSTYRVVSVLRIQHQDWLNGKKFKCSVNNQALPAPVERTISKATGQTRVPQVYVLAPHPDELSKNKVSVTCLVKDFYPPDITVEWQSNEHPEPEGKYRTTEAQKDSDGSYFLYSKLTVEKDRWQQGTTFTCVVMHEALHNHVMQKNISKNPGK

SEQ ID NO: 101 - protein, 214 aa, Artificial Sequence
  Synthetic Exemplary variant equine IgG4 Fc Protein A + C1q K(87)S
VGPSVFIFPPKPKDVLMISRTPTVTCVVVDVGHDFPDVQFNWYVDGVETHTATTEPKQEQFNSTYRVVSVLPIQHKDWLSGKEFKCSVNNKALPAPVERTISAPTGQPREPQVYVLAPHRDELSKNKVSVTCLVKDFYPPDIDIEWKSNGQPEPETKYSTTPAQLDSDGSYFLYSKLTVETNRWQQGTTFTCAVMHEALHNHYTEKSVSKSPGK

SEQ ID NO: 102 - protein, 214 aa, Artificial Sequence
  Synthetic Exemplary variant equine IgG7 Fc Protein A + C1q K(87)S
VGPSVFIFPPKPKDVLMISRTPTVTCVVVDVGHDFPDVQFNWYVDGVETHTATTEPKQEQNNSTYRVVSILAIQHKDWLSGKEFKCSVNNQALPAPVQKTISKPTGQPREPQVYVLAPHPDELSKNKVSVTCLVKDFYPPDIDIEWKSNGQPEPETKYSTTPAQLDGDGSYFLYSKLTVETNRWQQGTTFTCAVMHEALHNHYTEKSVSKSPGK

SEQ ID NO: 103 - protein, 237 aa, Artificial Sequence
  Synthetic Exemplary wild-type feline IgG1a Fc Protein A + C1q +
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 104 - protein, 237 aa, Artificial Sequence
  Synthetic Exemplary wild-type feline IgG1a Fc Protein A + C1q +
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 105 - protein, 237 aa, Artificial Sequence
  Synthetic Exemplary wild-type feline IgG1b Fc Protein A + C1q +
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 106 - protein, 237 aa, Artificial Sequence
  Synthetic Exemplary wild-type feline IgG1b Fc Protein A + C1q +
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 107 - protein, 237 aa, Artificial Sequence
  Synthetic Exemplary wild-type feline IgG2 Fc Protein A + C1q
PKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 108 - protein, 237 aa, Artificial Sequence; identical to SEQ ID NO: 104
  Synthetic Exemplary variant feline IgG1a Fc Protein A + C1q P(198)A

SEQ ID NO: 109 - protein, 237 aa, Artificial Sequence; identical to SEQ ID NO: 103
  Synthetic Exemplary variant feline IgG1a Fc Protein A + C1q P(198)A

SEQ ID NO: 110 - protein, 237 aa, Artificial Sequence; identical to SEQ ID NO: 106
  Synthetic Exemplary variant feline IgG1b Fc Protein A + C1q P(198)A

SEQ ID NO: 111 - protein, 237 aa, Artificial Sequence; identical to SEQ ID NO: 105
  Synthetic Exemplary variant feline IgG1b Fc Protein A + C1q P(198)A

SEQ ID NO: 112 - protein, 192 aa, Artificial Sequence
  Synthetic Exemplary feline EPO Analog 6-30 EV Precursor
MGSCECPALLLLLSLLLLPLGLPVLGAPPRLICDSRVLERYILEAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLCKLFRIYSNFLRGKLTLYTGEACRRGDR

SEQ ID NO: 113 - protein, 166 aa, Artificial Sequence
  Synthetic Exemplary feline EPO Analog 6-30 EV Mature
APPRLICDSRVLERYILEAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLCKLFRIYSNFLRGKLTLYTGEACRRGDR

SEQ ID NO: 114 - protein, 192 aa, Artificial Sequence
  Synthetic Exemplary feline EPO analog 14precursor D81N G83T
MGSCECPALLLLLSLLLLPLGLPVLGAPPRLICDSRVLERYILEAREAENVTMGCAEGCSFSENITVPDTKVNFYTWKRMNVTQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 115 - protein, 166 aa, Artificial Sequence
  Synthetic Exemplary feline EPO analog 14 mature D55N G57T
APPRLICDSRVLERYILEAREAENVTMGCAEGCSFSENITVPDTKVNFYTWKRMNVTQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 116 - protein, 192 aa, Artificial Sequence
  Synthetic Exemplary feline EPO analog 40 precursor L138N A140T
MGSCECPALLLLLSLLLLPLGLPVLGAPPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRANGTQKEATSLPEATSAAPLRTFTVDTLCKLFRIYSNFLRGKLTLYTGEACRRGDR

SEQ ID NO: 117 - protein, 166 aa, Artificial Sequence
  Synthetic Exemplary feline EPO analog 40 mature L112N A114T
APPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRANGTQKEATSLPEATSAAPLRTFTVDTLCKLFRIYSNFLRGKLTLYTGEACRRGDR

SEQ ID NO: 118 - protein, 192 aa, Artificial Sequence
  Synthetic Exemplary feline EPO analog 56 precursor L147N P148V E149T
MGSCECPALLLLLSLLLLPLGLPVLGAPPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSNVTATSAAPLRTFTVDTLCKLFRIYSNFLRGKLTLYTGEACRRGDR

SEQ ID NO: 119 - protein, 166 aa, Artificial Sequence
  Synthetic Exemplary feline EPO analog 56 mature L121N P122V E123T
APPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSNVTATSAAPLRTFTVDTLCKLFRIYSNFLRGKLTLYTGEACRRGDR

SEQ ID NO: 120 - protein, 192 aa, Artificial Sequence
  Synthetic Exemplary feline EPO analog 71 precursor P148E E149N
MGSCECPALLLLLSLLLLPLGLPVLGAPPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLENATSAAPLRTFTVDTLCKLFRIYSNFLRGKLTLYTGEACRRGDR

SEQ ID NO: 121 - protein, 166 aa, Artificial Sequence
  Synthetic Exemplary feline EPO analog 71 mature P122E E123N
APPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLENATSAAPLRTFTVDTLCKLFRIYSNFLRGKLTLYTGEACRRGDR

SEQ ID NO: 122 - protein, 192 aa, Artificial Sequence
  Synthetic Exemplary feline EPO analog precursor C165X
  misc_feature (165)..(165): wherein X may be any amino acid other than C, such as S, T, or A
MGSCECPALLLLLSLLLLPLGLPVLGAPPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLXKLFRIYSNFLRGKLTLYTGEACRRGDR

SEQ ID NO: 123 - protein, 166 aa, Artificial Sequence
  Synthetic Exemplary feline EPO analog mature C139X
  misc_feature (139)..(139): X may be any amino acid other than C, such as S, T, or A
APPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLXKLFRIYSNFLRGKLTLYTGEACRRGDR

(19) United States
(12) Patent Application Publication
(10) Pub. No.: US 2022/0025005 A1
(43) Pub. Date: Jan. 27, 2022

(54) Erythropoietin Analogs For Veterinary Use

(71) Applicant: Kindred Biosciences, Inc., Burlingame, CA (US)

(72) Inventors: Hangjun Zhan, Foster City, CA (US); Lam Nguyen, Union City, CA (US); Qingyi Chu, Burlingame, CA (US); Shyr Jiann Li, Millbrae, CA (US)

(73) Assignee: Kindred Biosciences, Inc., Burlingame, CA (US)

(21) Appl. No.: 17/299,841

(22) PCT Filed: Dec. 12, 2019
(86) PCT No.: PCT/US2019/066052

§ 371 Date: Jun. 4, 2021

Related U.S. Application Data
(60) Provisional application No. 62/778,849, filed on Dec. 12, 2018.
(60) Provisional application No. 62/779,332, filed on Dec. 13, 2018.
(60) Provisional application No. 62/785,691, filed on Dec. 27, 2018.

(51) Int. Cl. C07K 14/505; C07K 14/71; C07K 16/00

(52) U.S. Cl. CPC C07K 14/505; C07K 14/71; C07K 16/00; C07K 2317/524; A61K 38/1709; C07K 2317/71; C07K 2317/732; C07K 2317/734; C07K 2319/30; C07K 2317/526

(57) ABSTRACT

Provided are various embodiments relating to erythropoietin (EPO) polypeptide analogs with one or more additional glycosylation sites and/or additional cysteine residues and methods of producing and using the same to treat anemia in companion animals. Also provided are various embodiments relating to polypeptides comprising an extracellular domain of EPO receptor and methods of using the same for treating polycythemia in mammals.

### CROSS-REFERENCE TO RELATED APPLICATIONS

This application claims the benefit of priority to US Provisional Application Nos. 62/778,849, filed Dec. 12, 2018; 62/779,332, filed on Dec. 13, 2018; and 62/785,691, filed on Dec. 27, 2018, each of which is incorporated by reference herein in its entirety for any purpose.

### FIELD

This present disclosure relates to erythropoietin (EPO) polypeptide analogs having enhanced pharmacokinetics and methods of producing and using the same, for example, for treating anemia or hypoxia-related symptoms, such as chronic kidney disease (CDK), in companion animals, such as canines, felines, and equines. The present disclosure relates to nucleic acids, vectors, and expression systems encoding EPO polypeptides and methods of using the same (e.g., gene therapy methods), for example for controlled or induced expression of EPO polypeptides. This present disclosure further relates to formulations for the EPO polypeptides described herein. The present disclosure also relates to polypeptides comprising an extracellular domain of EPO receptor (EPOR) and methods of using the same, for example, for treating overproduction of EPO in companion animals.

### BACKGROUND

Erythropoietin (EPO), also known as hematopoietin or hemopoietin, is a glycoprotein hormone that can stimulate erythropoiesis (i.e., red blood cell production). EPO is used for treating anemia resulting from chronic kidney disease, inflammatory bowel disease (Crohn's disease and ulcer colitis) and myelodysplasia resulting from chemotherapy and radiation therapy. These human disorders are sometimes treated with a recombinant EPO molecule (e.g., Darbepoetin (Aranesp™ and Epogen™, Amgen) and Dynepo™ (Shire).

Companion animals suffer from many diseases that are similar to human diseases, including autoimmune diseases and cancer. While human proteins have been used to treat companion animal diseases, it is understood that proteins having significant human-derived amino acid sequence content can be immunogenic to the treated animals. If a human drug elicits an immune response in a companion animal, it may not be effective. See Mauldin et al., August 2010, 21(4):373-382.

Anemia in companion animals is currently treated by administering human erythropoietin drugs, such as Epogen™ or Aranesp™. However, it is likely that human EPO drugs could illicit an immunogenic response when administered to companion animals. In addition, human EPO drugs may not bind companion animal EPO receptor in a manner that provides an equally beneficial therapeutic effect in the companion animal as it does in humans.

There remains an unmet need, therefore, for methods and compounds that can be used to treat anemia (e.g., non-refractory anemia) in companion animals, including cats, dogs, and horses. Ideally, the compounds would bind specifically to EPO receptor and have a half-life in plasma sufficiently long to be practicable for therapy, but would be species specific and not be highly immunogenic. EPO polypeptides having enhanced pharmacokinetics and methods of administering those EPO polypeptides or nucleic acids encoding those EPO polypeptides for the treatment of anemia in companion animals are described herein.

Over production of EPO is also an issue. For example, polycythemia may be caused by overproduction and/or secretion of EPO from a tumor (e.g., a kidney tumor), by non-activating mutations in JAK2, or by a genetically-inherited dysregulation resulting in overproduction of EPO. Polypeptides comprising an extracellular domain of an EPOR and methods of administering those polypeptides or nucleic acids encoding those EPOR polypeptides for the treatment of polycythemia in companion animals.

### SUMMARY

Embodiment 1. An erythropoietin (EPO) polypeptide comprising the amino acid sequence of SEQ ID NO: 1, SEQ ID NO: 2, SEQ ID NO: 3, SEQ ID NO: 4, SEQ ID NO: 7, or SEQ ID NO: 8, except for the presence of at least one N-linked glycosylation site not present in SEQ ID NO: 1, SEQ ID NO: 2, SEQ ID NO: 3, SEQ ID NO: 4, SEQ ID NO: 7, or SEQ ID NO: 8, wherein the N-linked glycosylation site comprises the sequence asparagine-xaa-serine or asparagine-xaa-threonine, wherein xaa is any amino acid except proline, and wherein one N-linked glycosylation site does not overlap with another N-linked glycosylation site.

Embodiment 2. The EPO polypeptide of embodiment 1, wherein each of the at least one N-linked glycosylation sites is present at:
a) a position selected from position 47-49, 55-57, 56-58, 60-62, 61-63, 79-81, 81-83, 82-84, 91-93, 92-94, 97-99, 98-100, 99-101, 112-114, 113-115, 114-116, 115-117, 116-118, 137-139, 138-140, 140-142, 141-143, 142-144, 143-145, 144-146, 145-147, 146-148, 147-149, 148-150, 149-151, 150-152, 161-163, 162-164, 184-186, and 186-188 of SEQ ID NO: 1, SEQ ID NO: 3, or SEQ ID NO: 7; or
b) a position selected from position 21-23, 29-31, 30-32, 34-36, 35-37, 53-55, 55-57, 56-58, 65-67, 66-68, 71-73, 72-74, 73-75, 86-88, 87-89, 88-90, 89-91, 90-92, 111-113, 112-114, 114-116, 115-117, 116-118, 117-119, 118-120, 119-121, 120-122, 121-123, 122-124, 123-125, 124-126, 135-137, 136-138, 158-160, and 162-164 of SEQ ID NO: 2, or SEQ ID NO: 4, or SEQ ID NO: 8.

Embodiment 3. The EPO polypeptide of embodiment 1 or embodiment 2 comprising an amino acid except proline at a position corresponding to position 113 or position 148 of SEQ ID NO: 1, SEQ ID NO: 3, or SEQ ID NO: 7, or at a position corresponding to position 87 or position 122 of SEQ ID NO: 2, SEQ ID NO: 4, or SEQ ID NO: 8.

Embodiment 4. The EPO polypeptide of any one of embodiments 1 to 3 comprising valine or glutamic acid at a position corresponding to position 113 or position 148 of SEQ ID NO: 1, SEQ ID NO: 3, or SEQ ID NO: 7, or at a position corresponding to position 87 or position 122 of SEQ ID NO: 2, SEQ ID NO: 4, or SEQ ID NO: 8.

Embodiment 5. The EPO polypeptide of any one of embodiments 1 to 4 comprising:
a) asparagine at a position corresponding to position 47 of SEQ ID NO: 1, SEQ ID NO: 3, or SEQ ID NO: 7, any amino acid except proline at a position corresponding to position 48 of SEQ ID NO: 1, SEQ ID NO: 3, or SEQ ID NO: 7, and serine or threonine at a position corresponding to position 49 of SEQ ID NO: 1, SEQ ID NO: 3, or SEQ ID NO: 7; or
b) asparagine at a position corresponding to position 21 of SEQ ID NO: 2, SEQ ID NO: 4, or SEQ ID NO: 8, any amino acid except proline at a position corresponding to position 22 of SEQ ID NO: 2, SEQ ID NO: 4, or SEQ ID NO: 8, and serine or threonine at a position corresponding to position 23 of SEQ ID NO: 2, SEQ ID NO: 4, or SEQ ID NO: 8.

Embodiment 6. The EPO polypeptide of any one of embodiments 1 to 5 comprising:
a) asparagine at a position corresponding to position 55 of SEQ ID NO: 1, SEQ ID NO: 3, or SEQ ID NO: 7, any amino acid except proline at a position corresponding to position 56 of SEQ ID NO: 1, SEQ ID NO: 3, or SEQ ID NO: 7, and serine or threonine at a position corresponding to position 57 of SEQ ID NO: 1, SEQ ID NO: 3, or SEQ ID NO: 7; or
b) asparagine at a position corresponding to position 29 of SEQ ID NO: 2, SEQ ID NO: 4, or SEQ ID NO: 8, any amino acid except proline at a position corresponding to position 30 of SEQ ID NO: 2, SEQ ID NO: 4, or SEQ ID NO: 8, and serine or threonine at a position corresponding to position 31 of SEQ ID NO: 2, SEQ ID NO: 4, or SEQ ID NO: 8.

Embodiment 7. The EPO polypeptide of any one of embodiments 1 to 6 comprising:
a) asparagine at a position corresponding to position 56 of SEQ ID NO: 1, SEQ ID NO: 3, or SEQ ID NO: 7, any amino acid except proline at a position corresponding to position 57 of SEQ ID NO: 1, SEQ ID NO: 3, or SEQ ID NO: 7, and serine or threonine at a position corresponding to position 58 of SEQ ID NO: 1, SEQ ID NO: 3, or SEQ ID NO: 7; or
b) asparagine at a position corresponding to position 30 of SEQ ID NO: 2, SEQ ID NO: 4, or SEQ ID NO: 8, any amino acid except proline at a position corresponding to position 31 of SEQ ID NO: 2, SEQ ID NO: 4, or SEQ ID NO: 8, and serine or threonine at a position corresponding to position 32 of SEQ ID NO: 2, SEQ ID NO: 4, or SEQ ID NO: 8.

Embodiment 8. The EPO polypeptide of any one of embodiments 1 to 7 comprising:
a) asparagine at a position corresponding to position 60 of SEQ ID NO: 1, SEQ ID NO: 3, or SEQ ID NO: 7, any amino acid except proline at a position corresponding to position 61 of SEQ ID NO: 1, SEQ ID NO: 3, or SEQ ID NO: 7, and serine or threonine at a position corresponding to position 62 of SEQ ID NO: 1, SEQ ID NO: 3, or SEQ ID NO: 7; or
b) asparagine at a position corresponding to position 34 of SEQ ID NO: 2, SEQ ID NO: 4, or SEQ ID NO: 8, any amino acid except proline at a position corresponding to position 35 of SEQ ID NO: 2, SEQ ID NO: 4, or SEQ ID NO: 8, and serine or threonine at a position corresponding to position 36 of SEQ ID NO: 2, SEQ ID NO: 4, or SEQ ID NO: 8.

Embodiment 9. The EPO polypeptide of any one of embodiments 1 to 8 comprising:
a) asparagine at a position corresponding to position 61 of SEQ ID NO: 1, SEQ ID NO: 3, or SEQ ID NO: 7, any amino acid except proline at a position corresponding to position 62 of SEQ ID NO: 1, SEQ ID NO: 3, or SEQ ID NO: 7, and serine or threonine at a position corresponding to position 63 of SEQ ID NO: 1, or SEQ ID NO: 3, or SEQ ID NO: 7; or
b) asparagine at a position corresponding to position 35 of SEQ ID NO: 2, SEQ ID NO: 4, or SEQ ID NO: 8, any amino acid except proline at a position corresponding to position 36 of SEQ ID NO: 2, SEQ ID NO: 4 or SEQ ID NO: 8, and serine or threonine at a position corresponding to position 37 of SEQ ID NO: 2, SEQ ID NO: 4, or SEQ ID NO: 8.

Embodiment 10. The EPO polypeptide of any one of embodiments 1 to 9 comprising:
a) asparagine at a position corresponding to position 79 of SEQ ID NO: 1, SEQ ID NO: 3, or SEQ ID NO: 7, any amino acid except proline at a position corresponding to position 80 of SEQ ID NO: 1, SEQ ID NO: 3, or SEQ ID NO: 7, and serine or threonine at a position corresponding to position 81 of SEQ ID NO: 1, SEQ ID NO: 3, or SEQ ID NO: 7; or
b) asparagine at a position corresponding to position 53 of SEQ ID NO: 2, SEQ ID NO: 4, or SEQ ID NO: 8, any amino acid except proline at a position corresponding to position 54 of SEQ ID NO: 2, SEQ ID NO: 4, or SEQ ID NO: 8, and serine or threonine at a position corresponding to position 55 of SEQ ID NO: 2, SEQ ID NO: 4, or SEQ ID NO: 8.

Embodiment 11. The EPO polypeptide of any one of embodiments 1 to 10 comprising:
a) asparagine at a position corresponding to position 81 of SEQ ID NO: 1, SEQ ID NO: 3 or SEQ ID NO: 7, any amino acid except proline at a position corresponding to position 82 of SEQ ID NO: 1, SEQ ID NO: 3, or SEQ ID NO: 7, and serine or threonine at a position corresponding to position 83 of SEQ ID NO: 1, SEQ ID NO: 3, or SEQ ID NO: 7; or
b) asparagine at a position corresponding to position 55 of SEQ ID NO: 2, SEQ ID NO: 4, or SEQ ID NO: 8, any amino acid except proline at a position corresponding to position 56 of SEQ ID NO: 2, SEQ ID NO: 4, or SEQ ID NO: 8, and serine or threonine at a position corresponding to position 57 of SEQ ID NO: 2, SEQ ID NO: 4, or SEQ ID NO: 8.

Embodiment 12. The EPO polypeptide of any one of embodiments 1 to 11 comprising:
a) asparagine at a position corresponding to position 82 of SEQ ID NO: 1, SEQ ID NO: 3, or SEQ ID NO: 7, any amino acid except proline at a position corresponding to position 83 of SEQ ID NO: 1, SEQ ID NO: 3, or SEQ ID NO: 7, and serine or threonine at a position corresponding to position 84 of SEQ ID NO: 1, SEQ ID NO: 3, or SEQ ID NO: 7; or
b) asparagine at a position corresponding to position 56 of SEQ ID NO: 2, SEQ ID NO: 4, or SEQ ID NO: 8, any amino acid except proline at a position corresponding to position 57 of SEQ ID NO: 2, SEQ ID NO: 4, or SEQ ID NO: 8, and serine or threonine at a position corresponding to position 58 of SEQ ID NO: 2, SEQ ID NO: 4, or SEQ ID NO: 8.

Embodiment 13. The EPO polypeptide of any one of embodiments 1 to 12 comprising:
a) asparagine at a position corresponding to position 91 of SEQ ID NO: 1, SEQ ID NO: 3, or SEQ ID NO: 7, any amino acid except proline at a position corresponding to position 92 of SEQ ID NO: 1, SEQ ID NO: 3, or SEQ ID NO: 7, and serine or threonine at a position corresponding to position 93 of SEQ ID NO: 1, SEQ ID NO: 3, or SEQ ID NO: 8; or
b) asparagine at a position corresponding to position 65 of SEQ ID NO: 2, SEQ ID NO: 4, or SEQ ID NO: 8, any amino acid except proline at a position corresponding to position 66 of SEQ ID NO: 2, SEQ ID NO: 4, or SEQ ID NO: 8, and serine or threonine at a position corresponding to position 67 of SEQ ID NO: 2, SEQ ID NO: 4, or SEQ ID NO: 8.

Embodiment 14. The EPO polypeptide of any one of embodiments 1 to 13 comprising:
a) asparagine at a position corresponding to position 92 of SEQ ID NO: 1, SEQ ID NO: 3, or SEQ ID NO: 7, any amino acid except proline at a position corresponding to position 93 of SEQ ID NO: 1, SEQ ID NO: 3, or SEQ ID NO: 7, and serine or threonine at a position corresponding to position 94 of SEQ ID NO: 1, SEQ ID NO: 3, or SEQ ID NO: 7; or
b) asparagine at a position corresponding to position 66 of SEQ ID NO: 2, SEQ ID NO: 4, or SEQ ID NO: 8, any amino acid except proline at a position corresponding to position 67 of SEQ ID NO: 2, SEQ ID NO: 4, or SEQ ID NO: 8, and serine or threonine at a position corresponding to position 68 of SEQ ID NO: 2, SEQ ID NO: 4, or SEQ ID NO: 8.

Embodiment 15. The EPO polypeptide of any one of embodiments 1 to 14 comprising:
a) asparagine at a position corresponding to position 97 of SEQ ID NO: 1, SEQ ID NO: 3, or SEQ ID NO: 7, any amino acid except proline at a position corresponding to position 98 of SEQ ID NO: 1, SEQ ID NO: 3, or SEQ ID NO: 7, and serine or threonine at a position corresponding to position 99 of SEQ ID NO: 1, SEQ ID NO: 3, or SEQ ID NO: 7; or
b) asparagine at a position corresponding to position 71 of SEQ ID NO: 2, SEQ ID NO: 4, or SEQ ID NO: 8, any amino acid except proline at a position corresponding to position 92 of SEQ ID NO: 2, SEQ ID NO: 4, or SEQ ID NO: 8, and serine or threonine at a position corresponding to position 73 of SEQ ID NO: 2, SEQ ID NO: 4, or SEQ ID NO: 8.

Embodiment 16. The EPO polypeptide of any one of embodiments 1 to 15 comprising:
a) asparagine at a position corresponding to position 98 of SEQ ID NO: 1, SEQ ID NO: 3, or SEQ ID NO: 7, any amino acid except proline at a position corresponding to position 99 of SEQ ID NO: 1, SEQ ID NO: 3, or SEQ ID NO: 7, and serine or threonine at a position corresponding to position 100 of SEQ ID NO: 1, SEQ ID NO: 3, or SEQ ID NO: 7; or
b) asparagine at a position corresponding to position 72 of SEQ ID NO: 2, SEQ ID NO: 4, or SEQ ID NO: 8, any amino acid except proline at a position corresponding to position 73 of SEQ ID NO: 2, SEQ ID NO: 4, or SEQ ID NO: 8, and serine or threonine at a position corresponding to position 74 of SEQ ID NO: 2, SEQ ID NO: 4, or SEQ ID NO: 8.

Embodiment 17. The EPO polypeptide of any one of embodiments 1 to 16 comprising:
a) asparagine at a position corresponding to position 99 of SEQ ID NO: 1, SEQ ID NO: 3, or SEQ ID NO: 7, any amino acid except proline at a position corresponding to position 100 of SEQ ID NO: 1, SEQ ID NO: 3, or SEQ ID NO: 7, and serine or threonine at a position corresponding to position 101 of SEQ ID NO: 1, SEQ ID NO: 3, or SEQ ID NO: 7; or
b) asparagine at a position corresponding to position 73 of SEQ ID NO: 2, SEQ ID NO: 4, or SEQ ID NO: 8, any amino acid except proline at a position corresponding to position 74 of SEQ ID NO: 2, SEQ ID NO: 4, or SEQ ID NO: 8, and serine or threonine at a position corresponding to position 75 of SEQ ID NO: 2, SEQ ID NO: 4, or SEQ ID NO: 8.

Embodiment 18. The EPO polypeptide of any one of embodiments 1 to 17 comprising:
a) asparagine at a position corresponding to position 112 of SEQ ID NO: 1, SEQ ID NO: 3, or SEQ ID NO: 7, any amino acid except proline at a position corresponding to position 113 of SEQ ID NO: 1, SEQ ID NO: 3, or SEQ ID NO: 7, and serine or threonine at a position corresponding to position 114 of SEQ ID NO: 1, SEQ ID NO: 3, or SEQ ID NO: 7; or
b) asparagine at a position corresponding to position 86 of SEQ ID NO: 2, SEQ ID NO: 4, or SEQ ID NO: 8, any amino acid except proline at a position corresponding to position 87 of SEQ ID NO: 2, SEQ ID NO: 4, or SEQ ID NO: 8, and serine or threonine at a position corresponding to position 88 of SEQ ID NO: 2, SEQ ID NO: 4, or SEQ ID NO: 8.

Embodiment 19. The EPO polypeptide of any one of embodiments 1 to 18 comprising:
a) asparagine at a position corresponding to position 113 of SEQ ID NO: 1, SEQ ID NO: 3, or SEQ ID NO: 7, any amino acid except proline at a position corresponding to position 114 of SEQ ID NO: 1, SEQ ID NO: 3, or SEQ ID NO: 7, and serine or threonine at a position corresponding to position 115 of SEQ ID NO: 1, SEQ ID NO: 3, or SEQ ID NO: 7; or
b) asparagine at a position corresponding to position 87 of SEQ ID NO: 2, SEQ ID NO: 4, or SEQ ID NO: 8, any amino acid except proline at a position corresponding to position 88 of SEQ ID NO: 2, SEQ ID NO: 4, or SEQ ID NO: 8, and serine or threonine at a position corresponding to position 89 of SEQ ID NO: 2, SEQ ID NO: 4, or SEQ ID NO: 8.

Embodiment 20. The EPO polypeptide of any one of embodiments 1 to 19 comprising:
a) an asparagine at a position corresponding to position 114 of SEQ ID NO: 1, SEQ ID NO: 3, or SEQ ID NO: 7, any amino acid except proline at a position corresponding to position 115 of SEQ ID NO: 1, SEQ ID NO: 3, or SEQ ID NO: 7, and serine or threonine at a position corresponding to position 116 of SEQ ID NO: 1, SEQ ID NO: 3, or SEQ ID NO: 7, and optionally any amino acid except proline at a position corresponding to position 113 of SEQ ID NO: 1, SEQ ID NO: 3, or SEQ ID NO: 7; or
b) an asparagine at a position corresponding to position 88 of SEQ ID NO: 2, SEQ ID NO: 4, or SEQ ID NO: 8, any amino acid except proline at a position corresponding to position 89 of SEQ ID NO: 2, SEQ ID NO: 4, or SEQ ID NO: 8, and serine or threonine at a position corresponding to position 90 of SEQ ID NO: 2, SEQ ID NO: 4, or SEQ ID NO: 8, and optionally any amino acid except proline at a position corresponding to position 87 of SEQ ID NO: 2, SEQ ID NO: 4, or SEQ ID NO: 8.

Embodiment 21. The EPO polypeptide of any one of embodiments 1 to 20 comprising:
a) asparagine at a position corresponding to position 115 of SEQ ID NO: 1, SEQ ID NO: 3, SEQ ID NO: 7, any amino acid except proline at a position corresponding to position 116 of SEQ ID NO: 1, SEQ ID NO: 3, or SEQ ID NO: 7, and serine or threonine at a position corresponding to position 117 of SEQ ID NO: 1, SEQ ID NO: 3, or SEQ ID NO: 7; or
b) asparagine at a position corresponding to position 89 of SEQ ID NO: 2, SEQ ID NO: 4, or SEQ ID NO: 8, any amino acid except proline at a position corresponding to position 90 of SEQ ID NO: 2, SEQ ID NO: 4, or SEQ ID NO: 8, and serine or threonine at a position corresponding to position 91 of SEQ ID NO: 2, SEQ ID NO: 4, or SEQ ID NO: 8.

Embodiment 22. The EPO polypeptide of any one of embodiments 1 to 21 comprising:
a) asparagine at a position corresponding to position 116 of SEQ ID NO: 1, SEQ ID NO: 3, or SEQ ID NO: 7, any amino acid except proline at a position corresponding to position 117 of SEQ ID NO: 1, SEQ ID NO: 3, or SEQ ID NO: 7, and serine or threonine at a position corresponding to position 118 of SEQ ID NO: 1, SEQ ID NO: 3, or SEQ ID NO: 7; or
b) asparagine at a position corresponding to position 90 of SEQ ID NO: 2, SEQ ID NO: 4, or SEQ ID NO: 8, any amino acid except proline at a position corresponding to position 91 of SEQ ID NO: 2, SEQ ID NO: 4, or SEQ ID NO: 8, and serine or threonine at a position corresponding to position 92 of SEQ ID NO: 2, SEQ ID NO: 4, or SEQ ID NO: 8.

Embodiment 23. The EPO polypeptide of any one of embodiments 1 to 22 comprising:
a) asparagine at a position corresponding to position 137 of SEQ ID NO: 1, SEQ ID NO: 3, or SEQ ID NO: 7, any amino acid except proline at a position corresponding to position 138 of SEQ ID NO: 1, SEQ ID NO: 3, or SEQ ID NO: 7, and serine or threonine at a position corresponding to position 139 of SEQ ID NO: 1, or SEQ ID NO: 3, or SEQ ID NO: 7; or
b) asparagine at a position corresponding to position 111 of SEQ ID NO: 2, SEQ ID NO: 4, or SEQ ID NO: 8, any amino acid except proline at a position corresponding to position 112 of SEQ ID NO: 2, SEQ ID NO: 4, or SEQ ID NO: 8, and serine or threonine at a position corresponding to position 113 of SEQ ID NO: 2, SEQ ID NO: 4, or SEQ ID NO: 8.

Embodiment 24. The EPO polypeptide of any one of embodiments 1 to 23 comprising:
a) asparagine at a position corresponding to position 138 of SEQ ID NO: 1, SEQ ID NO: 3, or SEQ ID NO: 7, any amino acid except proline at a position corresponding to position 139 of SEQ ID NO: 1, SEQ ID NO: 3, or SEQ ID NO: 7, and serine or threonine at a position corresponding to position 140 of SEQ ID NO: 1, SEQ ID NO: 3, or SEQ ID NO: 7; or
b) asparagine at a position corresponding to position 112 of SEQ ID NO: 2, SEQ ID NO: 4, or SEQ ID NO: 8, any amino acid except proline at a position corresponding to position 113 of SEQ ID NO: 2, SEQ ID NO: 4, or SEQ ID NO: 8, and serine or threonine at a position corresponding to position 114 of SEQ ID NO: 2, SEQ ID NO: 4, or SEQ ID NO: 8.

Embodiment 25. The EPO polypeptide of any one of embodiments 1 to 24 comprising:
a) asparagine at a position corresponding to position 140 of SEQ ID NO: 1, SEQ ID NO: 3, or SEQ ID NO: 7, any amino acid except proline at a position corresponding to position 141 of SEQ ID NO: 1, SEQ ID NO: 3, or SEQ ID NO: 7, and serine or threonine at a position corresponding to position 142 of SEQ ID NO: 1, SEQ ID NO: 3, or SEQ ID NO: 7; or
b) asparagine at a position corresponding to position 114 of SEQ ID NO: 2, SEQ ID NO: 4, or SEQ ID NO: 8, any amino acid except proline at a position corresponding to position 115 of SEQ ID NO: 2, SEQ ID NO: 4, or SEQ ID NO: 8, and serine or threonine at a position corresponding to position 116 of SEQ ID NO: 2, SEQ ID NO: 4, or SEQ ID NO: 8.

Embodiment 26. The EPO polypeptide of any one of embodiments 1 to 25 comprising:
a) asparagine at a position corresponding to position 141 of SEQ ID NO: 1, SEQ ID NO: 3, or SEQ ID NO: 7, any amino acid except proline at a position corresponding to position 142 of SEQ ID NO: 1, SEQ ID NO: 3, or SEQ ID NO: 7, and serine or threonine at a position corresponding to position 143 of SEQ ID NO: 1, SEQ ID NO: 3, or SEQ ID NO: 7; or
b) asparagine at a position corresponding to position 115 of SEQ ID NO: 2, SEQ ID NO: 4, or SEQ ID NO: 8, any amino acid except proline at a position corresponding to position 116 of SEQ ID NO: 2, SEQ ID NO: 4, or SEQ ID NO: 8, and serine or threonine at a position corresponding to position 117 of SEQ ID NO: 2, SEQ ID NO: 4, or SEQ ID NO: 8.

Embodiment 27. The EPO polypeptide of any one of embodiments 1 to 26 comprising:
a) asparagine at a position corresponding to position 142 of SEQ ID NO: 1, SEQ ID NO: 3, or SEQ ID NO: 7, any amino acid except proline at a position corresponding to position 143 of SEQ ID NO: 1, SEQ ID NO: 3, or SEQ ID NO: 7, and serine or threonine at a position corresponding to position 144 of SEQ ID NO: 1, SEQ ID NO: 3, or SEQ ID NO: 7; or
b) asparagine at a position corresponding to position 116 of SEQ ID NO: 2, SEQ ID NO: 4, or SEQ ID NO: 8, any amino acid except proline at a position corresponding to position 117 of SEQ ID NO: 2, SEQ ID NO: 4, or SEQ ID NO: 8, and serine or threonine at a position corresponding to position 118 of SEQ ID NO: 2, SEQ ID NO: 4, or SEQ ID NO: 8.

Embodiment 28. The EPO polypeptide of any one of embodiments 1 to 27 comprising:
a) asparagine at a position corresponding to position 143 of SEQ ID NO: 1, SEQ ID NO: 3, or SEQ ID NO: 7, any amino acid except proline at a position corresponding to position 144 of SEQ ID NO: 1, SEQ ID NO: 3, or SEQ ID NO: 7, and serine or threonine at a position corresponding to position 145 of SEQ ID NO: 1, SEQ ID NO: 3, or SEQ ID NO: 7; or
b) asparagine at a position corresponding to position 117 of SEQ ID NO: 2, SEQ ID NO: 4, or SEQ ID NO: 8, any amino acid except proline at a position corresponding to position 118 of SEQ ID NO: 2, SEQ ID NO: 4, or SEQ ID NO: 8, and serine or threonine at a position corresponding to position 119 of SEQ ID NO: 2, SEQ ID NO: 4, or SEQ ID NO: 8.

Embodiment 29. The EPO polypeptide of any one of embodiments 1 to 28 comprising:
a) asparagine at a position corresponding to position 144 of SEQ ID NO: 1, SEQ ID NO: 3, or SEQ ID NO: 7, any amino acid except proline at a position corresponding to position 145 of SEQ ID NO: 1, SEQ ID NO: 3, or SEQ ID NO: 7, and serine or threonine at a position corresponding to position 146 of SEQ ID NO: 1, SEQ ID NO: 3, or SEQ ID NO: 7; or
b) asparagine at a position corresponding to position 118 of SEQ ID NO: 2, SEQ ID NO: 4, or SEQ ID NO: 8, any amino acid except proline at a position corresponding to position 119 of SEQ ID NO: 2, SEQ ID NO: 4, or SEQ ID NO: 8, and serine or threonine at a position corresponding to position 120 of SEQ ID NO: 2, SEQ ID NO: 4, or SEQ ID NO: 8.

Embodiment 30. The EPO polypeptide of any one of embodiments 1 to 29 comprising:
a) asparagine at a position corresponding to position 145 of SEQ ID NO: 1, SEQ ID NO: 3, or SEQ ID NO: 7, any amino acid except proline at a position corresponding to position 146 of SEQ ID NO: 1, SEQ ID NO: 3, or SEQ ID NO: 7, and serine or threonine at a position corresponding to position 147 of SEQ ID NO: 1, SEQ ID NO: 3, or SEQ ID NO: 7; or
b) asparagine at a position corresponding to position 119 of SEQ ID NO: 2, SEQ ID NO: 4, or SEQ ID NO: 8, any amino acid except proline at a position corresponding to position 120 of SEQ ID NO: 2, SEQ ID NO: 4, or SEQ ID NO: 8, and serine or threonine at a position corresponding to position 121 of SEQ ID NO: 2, SEQ ID NO: 4, or SEQ ID NO: 8.

Embodiment 31. The EPO polypeptide of any one of embodiments 1 to 30 comprising:
a) asparagine at a position corresponding to position 146 of SEQ ID NO: 1, SEQ ID NO: 3, or SEQ ID NO: 7, any amino acid except proline at a position corresponding to position 147 of SEQ ID NO: 1, SEQ ID NO: 3, or SEQ ID NO: 7, and serine or threonine at a position corresponding to position 148 of SEQ ID NO: 1, SEQ ID NO: 3, or SEQ ID NO: 7; or
b) asparagine at a position corresponding to position 120 of SEQ ID NO: 2, SEQ ID NO: 4, or SEQ ID NO: 8, any amino acid except proline at a position corresponding to position 121 of SEQ ID NO: 2, SEQ ID NO: 4, or SEQ ID NO: 8, and serine or threonine at a position corresponding to position 122 of SEQ ID NO: 2, SEQ ID NO: 4, or SEQ ID NO: 8.

Embodiment 32. The EPO polypeptide of any one of embodiments 1 to 31 comprising:
a) asparagine at a position corresponding to position 147 of SEQ ID NO: 1, SEQ ID NO: 3, or SEQ ID NO: 7, any amino acid except proline at a position corresponding to position 148 of SEQ ID NO: 1, SEQ ID NO: 3, or SEQ ID NO: 7, and serine or threonine at a position corresponding to position 149 of SEQ ID NO: 1, SEQ ID NO: 3, or SEQ ID NO: 7; or
b) asparagine at a position corresponding to position 121 of SEQ ID NO: 2, SEQ ID NO: 4, or SEQ ID NO: 8, any amino acid except proline at a position corresponding to position 122 of SEQ ID NO: 2, SEQ ID NO: 4, or SEQ ID NO: 8, and serine or threonine at a position corresponding to position 123 of SEQ ID NO: 2, SEQ ID NO: 4, or SEQ ID NO: 8.

Embodiment 33. The EPO polypeptide of any one of embodiments 1 to 32 comprising:
a) asparagine at a position corresponding to position 148 of SEQ ID NO: 1, SEQ ID NO: 3, or SEQ ID NO: 7, any amino acid except proline at a position corresponding to position 149 of SEQ ID NO: 1, SEQ ID NO: 3, or SEQ ID NO: 7, and serine or threonine at a position corresponding to position 150 of SEQ ID NO: 1, SEQ ID NO: 3, or SEQ ID NO: 7; or
b) asparagine at a position corresponding to position 122 of SEQ ID NO: 2, SEQ ID NO: 4, or SEQ ID NO: 8, any amino acid except proline at a position corresponding to position 123 of SEQ ID NO: 2, SEQ ID NO: 4, or SEQ ID NO: 8, and serine or threonine at a position corresponding to position 124 of SEQ ID NO: 2, SEQ ID NO: 4, or SEQ ID NO: 8.

Embodiment 34. The EPO polypeptide of any one of embodiments 1 to 33 comprising:
a) asparagine at a position corresponding to position 149 of SEQ ID NO: 1, SEQ ID NO: 3, or SEQ ID NO: 7, any amino acid except proline at a position corresponding to position 150 of SEQ ID NO: 1, SEQ ID NO: 3, or SEQ ID NO: 7, and serine or threonine at a position corresponding to position 151 of SEQ ID NO: 1, SEQ ID NO: 3, or SEQ ID NO: 7; or
b) asparagine at a position corresponding to position 123 of SEQ ID NO: 2, SEQ ID NO: 4, or SEQ ID NO: 8, any amino acid except proline at a position corresponding to position 124 of SEQ ID NO: 2, SEQ ID NO: 4, or SEQ ID NO: 8, and serine or threonine at a position corresponding to position 125 of SEQ ID NO: 2, SEQ ID NO: 4, or SEQ ID NO: 8.

Embodiment 35. The EPO polypeptide of any one of embodiments 1 to 34 comprising:
a) asparagine at a position corresponding to position 150 of SEQ ID NO: 1, SEQ ID NO: 3, or SEQ ID NO: 7, any amino acid except proline at a position corresponding to position 151 of SEQ ID NO: 1, SEQ ID NO: 3, or SEQ ID NO: 7, and serine or threonine at a position corresponding to position 152 of SEQ ID NO: 1, SEQ ID NO: 3, or SEQ ID NO: 7; or
b) asparagine at a position corresponding to position 124 of SEQ ID NO: 2, SEQ ID NO: 4, or SEQ ID NO: 8, any amino acid except proline at a position corresponding to position 125 of SEQ ID NO: 2, SEQ ID NO: 4, or SEQ ID NO: 8, and serine or threonine at a position corresponding to position 126 of SEQ ID NO: 2, SEQ ID NO: 4, or SEQ ID NO: 8.

Embodiment 36. The EPO polypeptide of any one of embodiments 1 to 35 comprising:
a) asparagine at a position corresponding to position 161 of SEQ ID NO: 1, SEQ ID NO: 3, or SEQ ID NO: 7, any amino acid except proline at a position corresponding to position 162 of SEQ ID NO: 1, SEQ ID NO: 3, or SEQ ID NO: 7, and serine or threonine at a position corresponding to position 163 of SEQ ID NO: 1, SEQ ID NO: 3, or SEQ ID NO: 7; or
b) asparagine at a position corresponding to position 135 of SEQ ID NO: 2, SEQ ID NO: 4, or SEQ ID NO: 8, any amino acid except proline at a position corresponding to position 136 of SEQ ID NO: 2, SEQ ID NO: 4, or SEQ ID NO: 8, and serine or threonine at a position corresponding to position 137 of SEQ ID NO: 2, SEQ ID NO: 4, or SEQ ID NO: 8.

Embodiment 37. The EPO polypeptide of any one of embodiments 1 to 36 comprising:
a) asparagine at a position corresponding to position 162 of SEQ ID NO: 1, SEQ ID NO: 3, or SEQ ID NO: 7, any amino acid except proline at a position corresponding to position 163 of SEQ ID NO: 1, SEQ ID NO: 3, or SEQ ID NO: 7, and serine or threonine at a position corresponding to position 164 of SEQ ID NO: 1, SEQ ID NO: 3, or SEQ ID NO: 7; or
b) asparagine at a position corresponding to position 136 of SEQ ID NO: 2, SEQ ID NO: 4, or SEQ ID NO: 8, any amino acid except proline at a position corresponding to position 137 of SEQ ID NO: 2, SEQ ID NO: 4, or SEQ ID NO: 8, and serine or threonine at a position corresponding to position 138 of SEQ ID NO: 2, SEQ ID NO: 4, or SEQ ID NO: 8.

Embodiment 38. The EPO polypeptide of any one of embodiments 1 to 37 comprising:
a) asparagine at a position corresponding to position 184 of SEQ ID NO: 1, SEQ ID NO: 3, or SEQ ID NO: 7, any amino acid except proline at a position corresponding to position 185 of SEQ ID NO: 1, SEQ ID NO: 3, or SEQ ID NO: 7, and serine or threonine at a position corresponding to position 186 of SEQ ID NO: 1, SEQ ID NO: 3, or SEQ ID NO: 7; or
b) asparagine at a position corresponding to position 158 of SEQ ID NO: 2, SEQ ID NO: 4, or SEQ ID NO: 8, any amino acid except proline at a position corresponding to position 159 of SEQ ID NO: 2, SEQ ID NO: 4, or SEQ ID NO: 8, and serine or threonine at a position corresponding to position 160 of SEQ ID NO: 2, SEQ ID NO: 4, or SEQ ID NO: 8.

Embodiment 39. The EPO polypeptide of any one of embodiments 1 to 38 comprising:
a) asparagine at a position corresponding to position 186 of SEQ ID NO: 1, SEQ ID NO: 3, or SEQ ID NO: 7, any amino acid except proline at a position corresponding to position 187 of SEQ ID NO: 1, SEQ ID NO: 3, or SEQ ID NO: 7, and serine or threonine at a position corresponding to position 188 of SEQ ID NO: 1, SEQ ID NO: 3, or SEQ ID NO: 7; or
b) asparagine at a position corresponding to position 162 of SEQ ID NO: 2, SEQ ID NO: 4, or SEQ ID NO: 8, any amino acid except proline at a position corresponding to position 163 of SEQ ID NO: 2, SEQ ID NO: 4, or SEQ ID NO: 8, and serine or threonine at a position corresponding to position 164 of SEQ ID NO: 2, SEQ ID NO: 4, or SEQ ID NO: 8.

Embodiment 40. The EPO polypeptide of any one of embodiments 1 to 39 comprising the amino acid sequence of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121.

Embodiment 41. An EPO polypeptide comprising the amino acid sequence of SEQ ID NO: 1, SEQ ID NO: 2, SEQ ID NO: 3, SEQ ID NO: 4, SEQ ID NO: 7, or SEQ ID NO: 8 except for the presence of at least one cysteine not present in SEQ ID NO: 1, SEQ ID NO: 2, SEQ ID NO: 3, SEQ ID NO: 4, SEQ ID NO: 7, or SEQ ID NO: 8.

Embodiment 42. The EPO polypeptide of any one of embodiments 1 to 41 comprising:
a) a cysteine at position 45, 48, 49, 68, 86, 90, 92 120, 143, 144, and/or 172 of SEQ ID NO: 1, SEQ ID NO: 3, or SEQ ID NO: 7; or
b) a cysteine at position 19, 22, 23, 42, 60, 64, 66, 94, 117, 118, and/or 146 of SEQ ID NO: 2, SEQ ID NO: 4, or SEQ ID NO: 8.

Embodiment 43. The EPO polypeptide of embodiment 41 or embodiment 42 comprising:
a) a cysteine at position 45 and 172 of SEQ ID NO: 1, SEQ ID NO: 3, or SEQ ID NO: 7; or
b) a cysteine at position 19 and 146 of SEQ ID NO: 2, SEQ ID NO: 4, or SEQ ID NO: 8.

Embodiment 44. The EPO polypeptide of any one of embodiments 41 to 43 comprising:
a) a cysteine at position 48 and 120 of SEQ ID NO: 1, SEQ ID NO: 3, or SEQ ID NO: 7; or
b) a cysteine at position 22 and 94 of SEQ ID NO: 2, SEQ ID NO: 4, or SEQ ID NO: 8.

Embodiment 45. The EPO polypeptide of any one of embodiments 41 to 44 comprising:
a) a cysteine at position 49 and 172 of SEQ ID NO: 1, SEQ ID NO: 3, or SEQ ID NO: 7; or
b) a cysteine at position 23 and 146 of SEQ ID NO: 2, SEQ ID NO: 4, or SEQ ID NO: 8.

Embodiment 46. The EPO polypeptide of any one of embodiments 41 to 45 comprising:
a) a cysteine at position 68 and 92 of SEQ ID NO: 1, SEQ ID NO: 3, or SEQ ID NO: 7; or
b) a cysteine at position 42 and 66 of SEQ ID NO: 2, SEQ ID NO: 4, or SEQ ID NO: 8.

Embodiment 47. The EPO polypeptide of any one of embodiments 41 to 46 comprising:
a) a cysteine at position 90 and 144 of SEQ ID NO: 1, SEQ ID NO: 3, or SEQ ID NO: 7; or
b) a cysteine at position 64 and 118 of SEQ ID NO: 2, SEQ ID NO: 4, or SEQ ID NO: 8.

Embodiment 48. The EPO polypeptide of any one of embodiments 41 to 47 comprising:
a) a cysteine at position 86 and 143 of SEQ ID NO: 1, SEQ ID NO: 3, or SEQ ID NO: 7; or
b) a cysteine at position 60 and 117 of SEQ ID NO: 2, SEQ ID NO: 4, or SEQ ID NO: 8.

Embodiment 49. The EPO polypeptide of any one of embodiments 1 to 48 comprising the amino acid sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, or SEQ ID NO: 32.

Embodiment 50. The EPO polypeptide of any one of claims 1 to 49 comprising an amino acid other than a cysteine at a position corresponding to position 165 of SEQ ID NO: 7 or at a position corresponding to position 139 of SEQ ID NO: 8.

Embodiment 51. An EPO polypeptide comprising the amino acid sequence of SEQ ID NO: 7 or SEQ ID NO: 8 except for the presence of an amino acid other than a cysteine at position 165 of SEQ ID NO: 7 or at position 139 of SEQ ID NO: 8.

Embodiment 52. The EPO polypeptide of claim 50 or 51, wherein the amino acid other than a cysteine is a threonine, a serine, or an alanine.

Embodiment 53. The EPO polypeptide of any one of embodiments 1 to 52, wherein the N-linked glycosylation site comprises an amino acid derivative.

Embodiment 54. The EPO polypeptide of embodiment 53, wherein the amino acid derivative is an asparagine derivative, a serine derivative, or a threonine derivative.

Embodiment 55. The EPO polypeptide of any one of embodiments 1 to 54, wherein the EPO polypeptide is glycosylated.

Embodiment 56. The EPO polypeptide of any one of embodiments 1 to 55 comprising at least one glycan moiety attached to the N-linked glycosylation site.

Embodiment 57. The EPO polypeptide of any one of embodiments 1 to 56, wherein the EPO polypeptide is PEGylated.

Embodiment 58. The EPO polypeptide of any one of embodiments 1 to 57, wherein the EPO polypeptide is PEGylated at a glycan.

Embodiment 59. The EPO polypeptide of any one of embodiments 1 to 58, wherein the EPO polypeptide is PEGylated at a primary amine.

Embodiment 60. The EPO polypeptide of any one of embodiments 1 to 59, wherein the EPO polypeptide is PEGylated at the N-terminal alpha-amine.

Embodiment 61. A contiguous polypeptide comprising the EPO polypeptide of any one of embodiments 1 to 60, wherein the contiguous polypeptide comprises an IgG Fc polypeptide.

Embodiment 62. The contiguous polypeptide of embodiment 61, wherein the IgG Fc polypeptide is a wild-type IgG Fc polypeptide.

Embodiment 63. The contiguous polypeptide of embodiment 62, wherein the IgG Fc polypeptide is a variant IgG Fc polypeptide.

Embodiment 64. The contiguous polypeptide of any one of embodiments 60 to 63, wherein the IgG Fc polypeptide is a variant IgG Fc polypeptide comprising:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide; and/or
c) at least one amino acid modification relative to a wild-type IgG Fc polypeptide of a companion animal species, wherein the variant IgG Fc polypeptide has reduced binding affinity to CD16 relative to the wild-type IgG Fc polypeptide.

Embodiment 65. The contiguous polypeptide of any one of the embodiments 60 to 64, wherein the variant IgG Fc polypeptide binds to C1q and/or CD16 with a dissociation constant (K_{d}) of greater than 5×10⁻⁶ M, greater than 1×10⁻⁵ M, greater than 5×10⁻⁵ M, greater than 1×10⁻⁴M, greater than 5×10⁻⁴M, or greater than 1×10⁻³M, as measured by biolayer interferometry.

Embodiment 66. The contiguous polypeptide of any one of the embodiments 60 to 65, wherein the variant IgG Fc polypeptide binds to Protein A with a dissociation constant (K_{d}) of less than 5×10⁻⁶ M, less than 1×10⁻⁶ M, less than 5×10⁻⁷ M, less than 1×10⁻⁷ M, less than 5×10⁻⁸M, less than 1×10⁻⁸M, less than 5×10⁻⁹M, less than 1×10⁻⁹M, less than 5×10⁻¹⁰ M, less than 1×10⁻¹⁰ M, less than 5×10⁻¹¹ M, less than 1×10⁻¹¹M, less than 5×10⁻¹² M, or less than 1×10⁻¹² M, as measured by biolayer interferometry.

Embodiment 67. The contiguous polypeptide of any one of the embodiments 60 to 66, wherein the companion animal species is canine, feline, or equine.

Embodiment 68. The contiguous polypeptide of any one of the embodiments 60 to 67, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1a Fc, IgG1b Fc, or IgG2 Fc.

Embodiment 69. The contiguous polypeptide of any one of the embodiments 60 to 68, wherein the variant IgG Fc polypeptide comprises:
a) an amino acid substitution at a position corresponding to position 21, position 23, position 25, position 80, position 205, and/or position 207 of SEQ ID NO: 53;
b) an amino acid substitution at a position corresponding to position 21, position 23, and/or position 24 of SEQ ID NO: 56;
c) an amino acid substitution at a position corresponding to position 21, position 23, position 25, position 80, and/or position 207 of SEQ ID NO: 58;
d) an amino acid substitution at a position corresponding to position 15 and/or position 203 of SEQ ID NO: 88;
e) an amino acid substitution at a position corresponding to position 199 and/or position 200 of SEQ ID NO: 92; and/or
f) an amino acid substitution at a position corresponding to position 199, position 200, position 201, and/or position 202 of SEQ ID NO: 93.

Embodiment 70. The contiguous polypeptide of any one of the embodiments 60 to 69, wherein the variant IgG Fc polypeptide comprises:
a) an amino acid substitution at position 21, position 23, position 25, position 80, position 205, and/or position 207 of SEQ ID NO: 53;
b) an amino acid substitution at position 21, position 23, and/or position 24 of SEQ ID NO: 56;
c) an amino acid substitution at position 21, position 23, position 25, position 80, and/or position 207 of SEQ ID NO: 58;
d) an amino acid substitution at position 15 and/or position 203 of SEQ ID NO: 88;
e) an amino acid substitution at position 199 and/or position 200 of SEQ ID NO: 92; and/or
f) an amino acid substitution at position 199, position 200, position 201, and/or position 202 of SEQ ID NO: 93.

Embodiment 71. The contiguous polypeptide of any one of the embodiments 60 to 70, wherein the variant IgG Fc polypeptide comprises:
a) a threonine at a position corresponding to position 21, a leucine at a position corresponding to position 23, an alanine at a position corresponding to position 25, a glycine at a position corresponding to position 80, an alanine at a position corresponding to position 205, and/or a histidine at a position corresponding to position 207 of SEQ ID NO: 53;
b) a threonine at a position corresponding to position 21, a leucine at a position corresponding to position 23, and/or an isoleucine at a position corresponding to position 24 of SEQ ID NO: 56;
c) a threonine at a position corresponding to position 21, a leucine at a position corresponding to position 23, an alanine at a position corresponding to position 25, a glycine at a position corresponding to position 80, and/or a histidine at a position corresponding to position 207 of SEQ ID NO: 58;
d) a threonine or a valine at a position corresponding to position 15 and/or a tyrosine or a valine at a position corresponding to position 203 of SEQ ID NO: 88;
e) a leucine at a position corresponding to position 199 and/or a histidine at a position corresponding to position 200 of SEQ ID NO: 92; and/or
f) a leucine at a position corresponding to position 199, a histidine at a position corresponding to position 200, an asparagine at a position corresponding to position 201, and/or a histidine at a position corresponding to position 202 of SEQ ID NO: 93.

Embodiment 72. The contiguous polypeptide of any one of the embodiments 60 to 71, wherein the variant IgG Fc polypeptide comprises:
a) a threonine at position 21, a leucine at position 23, an alanine at position 25, a glycine at position 80, an alanine at position 205, and/or a histidine at position 207 of SEQ ID NO: 53;
b) a threonine at position 21, a leucine at position 23, and/or an isoleucine at position 24 of SEQ ID NO: 56;
c) a threonine at a position 21, a leucine at position 23, an alanine at position 25, a glycine at position 80, and/or a histidine at position 207 of SEQ ID NO: 58;
d) a threonine or a valine at position 15, and/or a tyrosine or a valine at position 203 of SEQ ID NO: 88;
e) a leucine at position 199 and/or a histidine at position 200 of SEQ ID NO: 92; and/or
f) a leucine at position 199, a histidine at position 200, an asparagine at position 201, and/or a histidine at position 202 of SEQ ID NO: 93.

Embodiment 73. The contiguous polypeptide of any one of the embodiments 60 to 72, wherein the variant IgG Fc polypeptide comprises:
a) an amino acid substitution at a position corresponding to position 93 of SEQ ID NO: 54 or SEQ ID NO: 56;
b) an amino acid substitution at a position corresponding to position 87 of SEQ ID NO: 87, SEQ ID NO: 90, SEQ ID NO: 91, or SEQ ID NO: 94; or
c) an amino acid substitution at a position corresponding to position 198 of SEQ ID NO: 103, SEQ ID NO: 104, SEQ ID NO: 105, or SEQ ID NO: 106.

Embodiment 74. The contiguous polypeptide of any one of the embodiments 60 to 73, wherein the variant IgG Fc polypeptide comprises:
a) an amino acid substitution at position 93 of SEQ ID NO: 54 or SEQ ID NO: 56;
b) an amino acid substitution at position 87 of SEQ ID NO: 87, SEQ ID NO: 90, SEQ ID NO: 91, or SEQ ID NO: 94; or
c) an amino acid substitution at position 198 of SEQ ID NO: 103, SEQ ID NO: 104, SEQ ID NO: 105, or SEQ ID NO: 106.

Embodiment 75. The contiguous polypeptide of any one of the embodiments 60 to 74, wherein the variant IgG Fc polypeptide comprises:
a) an arginine at a position corresponding to position 93 of SEQ ID NO: 54 or SEQ ID NO: 56;
b) a serine at a position corresponding to position 87 of SEQ ID NO: 87, SEQ ID NO: 90, SEQ ID NO: 91, or SEQ ID NO: 94; or
c) an alanine at a position corresponding to position 198 of SEQ ID NO: 103, SEQ ID NO: 104, SEQ ID NO: 105, or SEQ ID NO: 106.

Embodiment 76. The contiguous polypeptide of any one of the embodiments 60 to 75, wherein the variant IgG Fc polypeptide comprises:
a) an arginine at position 93 of SEQ ID NO: 54 or SEQ ID NO: 56;
b) a serine at position 87 of SEQ ID NO: 87, SEQ ID NO: 90, SEQ ID NO: 91, or SEQ ID NO: 94; or
c) an alanine at position 198 of SEQ ID NO: 103, SEQ ID NO: 104, SEQ ID NO: 105, or SEQ ID NO: 106.

Embodiment 77. The contiguous polypeptide of any one of the embodiments 60 to 76, wherein the variant IgG Fc polypeptide comprises:
a) an amino acid substitution at a position corresponding to position 5, position 38, position 39, position 97, and/or position 98 of SEQ ID NO: 54; or
b) an amino acid substitution at a position corresponding to position 5, position 38, position 39, position 97, and/or position 98 of SEQ ID NO: 56.

Embodiment 78. The contiguous polypeptide of any one of the embodiments 60 to 77, wherein the variant IgG Fc polypeptide comprises:
a) an amino acid substitution at position 5, position 38, position 39, position 97, and/or position 98 of SEQ ID NO: 54; or
b) an amino acid substitution at position 5, position 38, position 39, position 97, and/or position 98 of SEQ ID NO: 56.

Embodiment 79. The contiguous polypeptide of any one of the embodiments 60 to 78, wherein the variant IgG Fc polypeptide comprises:
a) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 54; or
b) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 56.

Embodiment 80. The contiguous polypeptide of any one of the embodiments 60 to 79, wherein the variant IgG Fc polypeptide comprises:
a) a proline at position 5, a glycine at position 38, an arginine at position 39, an isoleucine at position 97, and/or a glycine at position 98 of SEQ ID NO: 54; or
b) a proline at position 5, a glycine at position 38, an arginine at position 39, an isoleucine at position 97, and/or a glycine at position 98 of SEQ ID NO: 56.

Embodiment 81. The contiguous polypeptide of any one of embodiments 60 to 80, wherein the variant IgG Fc polypeptide comprises the amino acid sequence of SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, or SEQ ID NO: 111.

Embodiment 82. A composition comprising a plurality of EPO polypeptides of any one of embodiments 1 to 81 having a range of isoelectric points of from about 1 to about 3.5, of from about 1.5 to about 3.5, of from about 2 to about 3.5, of from about 2.5 to about 3.5, of from about 3 to about 3.5, of about 3.5 or less, or of about 3 or less, as determined by isoelectric focusing.

Embodiment 83. A composition comprising a plurality of EPO polypeptides of any one of embodiments 1 to 81 having a range of isoelectric points of from about 3.5 to about 6, of from about 4 to about 6, of from about 4.5 to about 6, of from about 5 to about 6, of from about 5.5 to about 6, of from about 3.5 to about 5, of from about 4 to about 5, of from about 4.5 to about 5, of about 3.5 or greater, of about 4 or greater, or of about 4.5 or greater, as determined by isoelectric focusing.

Embodiment 84. A combination comprising the composition of embodiment 82 and the composition of embodiment 83.

Embodiment 85. An isolated nucleic acid encoding the EPO polypeptide of any one of embodiments 1 to 81.

Embodiment 86. The nucleic acid of embodiment 85, wherein the nucleic acid comprises a regulatory sequence.

Embodiment 87. The nucleic acid of embodiment 86, wherein the regulatory sequence is a constitutive promoter; an inducible regulatory sequence, such as a tetracycline response element or a hypoxia-inducible promoter; a tissue specific promoter; an enhancer; a silencer; or encodes a micro RNA or transcription factor.

Embodiment 88. An isolated nucleic acid encoding an EPO polypeptide comprising the amino acid sequence of SEQ ID NO: 1, SEQ ID NO: 2, SEQ ID NO: 3, or SEQ ID NO: 4; and a heterologous regulatory sequence, wherein the heterologous regulatory sequence is not a constitutive promoter.

Embodiment 89. The nucleic acid of embodiment 88, wherein the heterologous regulatory sequence is an inducible regulatory sequence, such as a tetracycline response element or a hypoxia-inducible promoter; a tissue specific promoter; an enhancer; a silencer; or encodes a micro RNA or transcription factor.

Embodiment 90. A vector comprising the nucleic acid of any one of embodiments 86 to 89.

Embodiment 91. The vector of embodiment 90, wherein the vector is a viral vector or a bacterial vector.

Embodiment 92. The vector of embodiment 90 or embodiment 91, wherein the vector is a retroviral vector, a herpesviral vector, an adenoviral vector, an adeno-associated viral vector, or a pox viral vector.

Embodiment 93. An expression system comprising a first vector comprising a nucleic acid encoding the EPO polypeptide of any one of embodiments 1 to 81; and a second vector comprising a regulatory sequence.

Embodiment 94. An expression system comprising a first vector comprising a nucleic acid encoding an EPO polypeptide comprising the amino acid sequence of SEQ ID NO: 1, SEQ ID NO: 2, SEQ ID NO: 3, or SEQ ID NO: 4; and a second vector comprising a regulatory sequence.

Embodiment 95. The expression system of embodiment 93 or embodiment 94, wherein the regulatory sequence encodes a micro RNA or transcription factor.

Embodiment 96. The expression system of any one of embodiments 93 to 95, wherein the first vector and/or second vector is a viral vector or a bacterial vector.

Embodiment 97. The expression system of any one of embodiments 93 to 96, wherein the first vector and/or second vector is a retroviral vector, a herpesviral vector, an adenoviral vector, an adeno-associated viral vector, or a pox viral vector.

Embodiment 98. A host cell comprising the nucleic acid of any one of embodiments 85 to 89, the vector of any one of embodiments 90 to 92, or the expression system of any one of embodiments 94 to 97.

Embodiment 99. A method of producing a composition comprising EPO polypeptides comprising culturing the host cell of embodiment 98 and isolating the EPO polypeptides.

Embodiment 100. The method of embodiment 99, wherein the EPO polypeptides are isolated by column chromatography.

Embodiment 101. The method of embodiment 99 or embodiment 100, wherein the EPO polypeptides are isolated by ion exchange column chromatography.

Embodiment 102. The method of any one of embodiments 99 to 101, wherein the EPO polypeptides are isolated by Capto Butyl column chromatography, cation-exchange column chromatography, or anion-exchange column chromatography.

Embodiment 103. The method of any one of embodiments 99 to 102, wherein the EPO polypeptides are isolated by mixed-mode column chromatography.

Embodiment 104. The method of any one of embodiments 99 to 103, wherein the EPO polypeptides are isolated by hydrophobic interaction column chromatography.

Embodiment 105. The method of any one of embodiments 99 to 104, wherein the EPO polypeptides are isolated by a combination of chromatography columns.

Embodiment 106. The method of any one of embodiments 99 to 105, wherein the method further comprises inactivating and/or removing viruses.

Embodiment 107. The method of any one of embodiments 99 to 106, wherein the EPO polypeptides have a range of isoelectric points of from about 1 to about 3.5, of from about 1.5 to about 3.5, of from about 2 to about 3.5, of from about 2.5 to about 3.5, of from about 3 to about 3.5, of about 3.5 or less, or of about 3 or less, as determined by isoelectric focusing.

Embodiment 108. The method of any one of embodiments 99 to 106, wherein the EPO polypeptides have a range of isoelectric points of from about 3.5 to about 6, of from about 4 to about 6, of from about 4.5 to about 6, of from about 5 to about 6, of from about 5.5 to about 6, of from about 3.5 to about 5, of from about 4 to about 5, of from about 4.5 to about 5, of about 3.5 or greater, of about 4 or greater, or of about 4.5 or greater, as determined by isoelectric focusing.

Embodiment 109. A pharmaceutical composition comprising the EPO polypeptide of any one of embodiments 1 to 81, the composition of embodiment 82 or embodiment 83, the combination of embodiment 84, the nucleic acid of any one of embodiments 85 to 89, the vector of any one of embodiments 90 to 92, or the expression system of any one of embodiments 93 to 97, and a pharmaceutically acceptable carrier.

Embodiment 110. A pharmaceutical composition comprising the EPO polypeptide of any one of embodiments 1 to 81, the composition of embodiment 82 or embodiment 83, or the combination of embodiment 84 and a pharmaceutically acceptable carrier, wherein the pharmaceutically acceptable carrier comprises a) sodium phosphate, sodium chloride, and polysorbate 80; b) sodium phosphate, sodium chloride, and polysorbate 20; c) sodium citrate, sodium chloride, and polysorbate 80; or d) sodium citrate, sodium chloride, and polysorbate 20.

Embodiment 111. A pharmaceutical composition comprising the EPO polypeptide of any one of embodiments 1 to 81, the composition of embodiment 82 or embodiment 83, or the combination of embodiment 84 and a pharmaceutically acceptable carrier, wherein the pharmaceutically acceptable carrier comprises sodium citrate, sodium chloride, polysorbate 80, and m-cresol.

Embodiment 112. A pharmaceutical composition comprising the EPO polypeptide of any one of embodiments 1 to 81, the composition of embodiment 82 or embodiment 83, or the combination of embodiment 84 and a pharmaceutically acceptable carrier, wherein the pharmaceutically acceptable carrier comprises sodium phosphate, sodium chloride, polysorbate 20, and benzyl alcohol.

Embodiment 113. The pharmaceutical composition of any one of embodiments 110 to 112, wherein the concentration of sodium chloride is about 140 mM.

Embodiment 114. The pharmaceutical composition of any one of embodiments 110 to 113, wherein the concentration of sodium phosphate or sodium citrate is about 20 mM.

Embodiment 115. The pharmaceutical composition of any one of embodiments 110 to 114, wherein the concentration of polysorbate 20 or polysorbate 80 is about 650 nM.

Embodiment 116. The pharmaceutical composition of any one of embodiments 111, or 113 to 115, wherein the concentration of m-cresol is about 0.2%.

Embodiment 117. The pharmaceutical composition of any one of embodiments 112 to 116, wherein the concentration of benzyl alcohol is about 1%.

Embodiment 118. The pharmaceutical composition of any one of embodiments 110 to 117, wherein the pharmaceutically acceptable carrier comprises:
a) sodium phosphate at a concentration of about 20 mM, sodium chloride at a concentration of about 140 mM, polysorbate 80 at a concentration of about 650 nM or
b) sodium phosphate at a concentration of about 20 mM, sodium chloride at a concentration of about 140 mM, polysorbate 20 at a concentration of about 650 nM.

Embodiment 119. The pharmaceutical composition of any one of embodiments 110 to 118, wherein the pharmaceutically acceptable carrier comprises sodium citrate at a concentration of about 20 mM, sodium chloride at a concentration of about 140 nM, polysorbate 80 at a concentration of about 650 nM, and m-cresol at a concentration of about 0.2%.

Embodiment 120. The pharmaceutical composition of any one of embodiments 110 to 119, wherein the pharmaceutically acceptable carrier comprises sodium phosphate at a concentration of about 20 mM, sodium chloride at a concentration of about 140 nM, polysorbate 20 at a concentration of about 650 nM, and benzyl alcohol at a concentration of about 1%.

Embodiment 121. A method of delivering an EPO polypeptide to a companion animal species comprising administering the EPO polypeptide of any one of embodiments 1 to 81, the composition of embodiment 82 or embodiment 83, the combination of embodiment 84, or the pharmaceutical composition of any one of embodiments 109 to 120 parenterally.

Embodiment 122. A method of delivering an EPO polypeptide to a companion animal species comprising administering the EPO polypeptide of any one of embodiments 1 to 81, the composition of embodiment 82 or embodiment 83, the combination of embodiment 84, or the pharmaceutical composition of any one of embodiments 109 to 120 by an intramuscular route, an intraperitoneal route, an intracerebrospinal route, a subcutaneous route, an intra-arterial route, an intrasynovial route, an intrathecal route, or an inhalation route.

Embodiment 123. A method of delivering an isolated nucleic acid encoding an EPO polypeptide to a companion animal species comprising administering the nucleic acid of any one of embodiments 85 to 89, the vector of any one of embodiments 90 to 91, or the expression system of any one of embodiments 93 to 97 parenterally.

Embodiment 124. A method of treating a companion animal species having anemia comprising administering to the companion animal species a therapeutically effective amount of the EPO polypeptide of any one of embodiments 1 to 81, the composition of embodiments 82 or 83, the combination of embodiment 84, or the pharmaceutical composition of any one of embodiments 109 to 120.

Embodiment 125. A method of treating a companion animal species having anemia, the method comprising administering to the companion animal species a therapeutically effective amount of the nucleic acid of any one of embodiments 85 to 89, the vector of any one of embodiments 90 to 92, or the expression system of any one of embodiments 93 to 97.

Embodiment 126. The method of embodiment 124 or embodiment 125, wherein the EPO polypeptide, composition, nucleic acid, vector, expression system, or pharmaceutical composition is administered parenterally.

Embodiment 127. The method of any one of embodiments 124 to 126, wherein the EPO polypeptide, composition, nucleic acid, vector, expression system, or pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 128. The method of any one of embodiments 121 to 127, wherein the companion animal species is feline, canine, or equine.

Embodiment 129. The method of any one of embodiments 124 to 128, wherein the anemia is caused by chronic kidney disease, inflammatory bowel disease, or myelodysplasia.

Embodiment 130. The method of any one of embodiments 121 to 129, wherein the EPO polypeptide is administered in an amount of from about 1 μg/kg body weight to about 10 μg/kg body weight, or about 1 μg/kg body weight to about 5 μg/kg body weight, or about 1 μg/kg body weight, or about 3 μg/kg body weight, or about 5 μg/kg body weight, or about 10 μg/kg body weight.

Embodiment 131. The method of any one of embodiments 121 to 130, wherein the EPO polypeptide, composition, nucleic acid, vector, expression system, or pharmaceutical composition is administered every 7 to 10 days.

Embodiment 132. The method of any one of embodiments 121 to 131, wherein the method comprises administering iron dextran.

Embodiment 133. The method of any one of embodiments 121 to 132, wherein the companion animal species has a baseline hematocrit percentage of from about 15% to about 30%, of from about 15% to about 25%, of from about 20% to about 25%, of from about 25% to about 30%, of below about 15%, of below about 18%, of below about 20%, of below about 25%, of below about 29%, or of below about 30% prior to administration of the EPO polypeptide, composition, nucleic acid, vector, expression system, or pharmaceutical composition.

Embodiment 134. The method of any one of embodiments 121 to 133, wherein the hematocrit percentage of the companion animal species increases to at least 25%, or at least 26%, or at least 27%, or at least 28%, or at least 29%, or at least 30%, or at least 32%, or at least 35%, or at least 38%, or at least 40%, or at least 42%, or at least 45%, or at least 48% following administration of the EPO polypeptide, composition, nucleic acid, vector, expression system, or pharmaceutical composition.

Embodiment 135. The method of embodiment 134, wherein the hematocrit percentage of the companion animal species increases to at least 25%, or at least 27%, or at least 30%, or at least 32%, or at least 35% at 1 week, 2 weeks, 3 weeks, 4 weeks, 5 weeks, or 6 weeks after a first administration of the EPO polypeptide, composition, nucleic acid, vector, expression system, or pharmaceutical composition.

Embodiment 136. The method of any one of embodiments 121 to 135, wherein the body weight of the companion animal species is maintained or increased compared to baseline following administration of the EPO polypeptide, composition, nucleic acid, vector, expression system, or pharmaceutical composition.

Embodiment 137. The method of embodiment 136, wherein the body weight of the companion animal species is maintained or increased at 1 week, 2 weeks, 3 weeks, 4 weeks, 5 weeks, or 6 weeks after a first administration of the EPO polypeptide, composition, nucleic acid, vector, expression system, or pharmaceutical composition.

Embodiment 138. The method of any one of embodiments 121 to 137, wherein the level of symmetric dimethylarginine or serum creatine renal biomarker is decreased compared to baseline following administration of the EPO polypeptide, composition, nucleic acid, vector, expression system, or pharmaceutical composition.

Embodiment 139. A method of expressing an EPO polypeptide in a target cell, comprising
a) transferring a nucleic acid, vector, or expression system into the target cell, wherein the nucleic acid, vector, or expression system comprises:
   i) a nucleic acid encoding the EPO polypeptide of any one of embodiments 1 to 81, and
   ii) a regulatory sequence; and
b) culturing the cell under conditions supportive for expression of the EPO polypeptide.

Embodiment 140. A method of expressing an EPO polypeptide in a target cell, comprising
a) transferring a nucleic acid, a vector, or an expression system into the target cell, wherein the nucleic acid, vector, or expression system comprises:
   i) a nucleic acid encoding an EPO polypeptide having the amino acid sequence of SEQ ID NO: 1, SEQ ID NO: 2, SEQ ID NO: 3, or SEQ ID NO: 4, and
   ii) a regulatory sequence, wherein the regulatory sequence is not a constitutive promoter; and
b) culturing the cell under conditions supportive for expression of the EPO polypeptide.

Embodiment 141. The method of embodiment 139 or embodiment 140, wherein the regulatory sequence is an inducible regulatory sequence, such as a tetracycline response element or a hypoxia-inducible promoter; a tissue specific promoter; an enhancer; a silencer; or encodes a micro RNA or transcription factor.

Embodiment 142. The method of any one of embodiments 139 to 141, wherein the vector is a viral vector or a bacterial vector.

Embodiment 143. The method of any one of embodiments 139 to 142, wherein the vector is a retroviral vector, a herpesviral vector, an adenoviral vector, an adeno-associated viral vector, or a pox viral vector.

Embodiment 144. The method of any one of embodiments 139 to 143, wherein the cell is a cell of a companion animal species.

Embodiment 145. The method of any one of embodiments 139 to 144, wherein the cell is located in a living companion animal species.

Embodiment 146. The method of embodiment 144 or embodiment 144, wherein the companion animal species is a canine, feline, or equine.

Embodiment 147. A polypeptide comprising an extracellular domain of a canine, equine, or feline erythropoietin receptor (EPOR) polypeptide, wherein the canine, equine, or feline EPOR polypeptide comprises the amino acid sequence of SEQ ID NO: 33, SEQ ID NO: 37, SEQ ID NO: 41, SEQ ID NO: 44, SEQ ID NO: 47, or SEQ ID NO: 50; and a heterologous polypeptide sequence.

Embodiment 148. A polypeptide comprising the amino acid sequence of SEQ ID NO: 34, SEQ ID NO: 35, SEQ ID NO: 38, SEQ ID NO: 39, SEQ ID NO: 42, SEQ ID NO: 43, SEQ ID NO: 45, SEQ ID NO: 46, SEQ ID NO: 48, SEQ ID NO: 49, SEQ ID NO: 51, or SEQ ID NO: 52; and a heterologous polypeptide sequence.

Embodiment 149. A contiguous polypeptide comprising the polypeptide of embodiment 147 or embodiment 148, wherein the contiguous polypeptide comprises an IgG Fc polypeptide.

Embodiment 150. The contiguous polypeptide of embodiment 149, wherein the IgG Fc polypeptide is a wild-type IgG Fc polypeptide.

Embodiment 151. The contiguous polypeptide of embodiment 149, wherein the IgG Fc polypeptide is a variant IgG Fc polypeptide.

Embodiment 152. The contiguous polypeptide of any one of embodiments 149 to 151, wherein the IgG Fc polypeptide is a variant IgG Fc polypeptide comprising:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide; and/or
c) at least one amino acid modification relative to a wild-type IgG Fc polypeptide of a companion animal species, wherein the variant IgG Fc polypeptide has reduced binding affinity to CD16 relative to the wild-type IgG Fc polypeptide.

Embodiment 153. The contiguous polypeptide of any one of the embodiments 149 to 152, wherein the variant IgG Fc polypeptide binds to C1q and/or CD16 with a dissociation constant (K_{d}) of greater than 5×10⁻⁶ M, greater than 1×10⁻⁵ M, greater than 5×10⁻⁵ M, greater than 1×10⁻⁴ M, greater than 5×10⁻⁴ M, or greater than 1×10⁻³M, as measured by biolayer interferometry.

Embodiment 154. The contiguous polypeptide of any one of the embodiments 149 to 153, wherein the variant IgG Fc polypeptide binds to Protein A with a dissociation constant (K_{d}) of less than 5×10⁻⁶ M, less than 1×10⁻⁶ M, less than 5×10⁻⁷ M, less than 1×10⁻⁷ M, less than 5×10⁻⁸M, less than 1×10⁻⁸M, less than 5×10⁻⁹M, less than 1×10⁻⁹M, less than 5×10⁻¹⁰ M, less than 1×10⁻¹⁰ M, less than 5×10⁻¹¹ M, less than 1×10⁻¹¹M, less than 5×10⁻¹² M, or less than 1×10⁻¹² M, as measured by biolayer interferometry.

Embodiment 155. The contiguous polypeptide of any one of the embodiments 149 to 154, wherein the companion animal species is canine, feline, or equine.

Embodiment 156. The contiguous polypeptide of any one of the embodiments 149 to 155, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1a Fc, IgG1b Fc, or IgG2 Fc.

Embodiment 157. The contiguous polypeptide of any one of the embodiments 149 to 156, wherein the variant IgG Fc polypeptide comprises:
a) an amino acid substitution at a position corresponding to position 21, position 23, position 25, position 80, position 205, and/or position 207 of SEQ ID NO: 53;
b) an amino acid substitution at a position corresponding to position 21, position 23, and/or position 24 of SEQ ID NO: 56;
c) an amino acid substitution at a position corresponding to position 21, position 23, position 25, position 80, and/or position 207 of SEQ ID NO: 58;
d) an amino acid substitution at a position corresponding to position 15 and/or position 203 of SEQ ID NO: 88;
e) an amino acid substitution at a position corresponding to position 199 and/or position 200 of SEQ ID NO: 92; and/or
f) an amino acid substitution at a position corresponding to position 199, position 200, position 201, and/or position 202 of SEQ ID NO: 93.

Embodiment 158. The contiguous polypeptide of any one of the embodiments 149 to 157, wherein the variant IgG Fc polypeptide comprises:
a) an amino acid substitution at position 21, position 23, position 25, position 80, position 205, and/or position 207 of SEQ ID NO: 53;
b) an amino acid substitution at position 21, position 23, and/or position 24 of SEQ ID NO: 56;
c) an amino acid substitution at position 21, position 23, position 25, position 80, and/or position 207 of SEQ ID NO: 58;
d) an amino acid substitution at position 15 and/or position 203 of SEQ ID NO: 88;
e) an amino acid substitution at position 199 and/or position 200 of SEQ ID NO: 92; and/or
f) an amino acid substitution at position 199, position 200, position 201, and/or position 202 of SEQ ID NO: 93.

Embodiment 159. The contiguous polypeptide of any one of the embodiments 149 to 158, wherein the variant IgG Fc polypeptide comprises:
a) a threonine at a position corresponding to position 21, a leucine at a position corresponding to position 23, an alanine at a position corresponding to position 25, a glycine at a position corresponding to position 80, an alanine at a position corresponding to position 205, and/or a histidine at a position corresponding to position 207 of SEQ ID NO: 53;
b) a threonine at a position corresponding to position 21, a leucine at a position corresponding to position 23, and/or an isoleucine at a position corresponding to position 24 of SEQ ID NO: 56;
c) a threonine at a position corresponding to position 21, a leucine at a position corresponding to position 23, an alanine at a position corresponding to position 25, a glycine at a position corresponding to position 80, and/or a histidine at a position corresponding to position 207 of SEQ ID NO: 58;
d) a threonine or a valine at a position corresponding to position 15 and/or a tyrosine or a valine at a position corresponding to position 203 of SEQ ID NO: 88;
e) a leucine at a position corresponding to position 199 and/or a histidine at a position corresponding to position 200 of SEQ ID NO: 92; and/or
f) a leucine at a position corresponding to position 199, a histidine at a position corresponding to position 200, an asparagine at a position corresponding to position 201, and/or a histidine at a position corresponding to position 202 of SEQ ID NO: 93.

Embodiment 160. The contiguous polypeptide of any one of embodiments 149 to 159, wherein the variant IgG Fc polypeptide comprises:
a) a threonine at position 21, a leucine at position 23, an alanine at position 25, a glycine at position 80, an alanine at position 205, and/or a histidine at position 207 of SEQ ID NO: 53;
b) a threonine at position 21, a leucine at position 23, and/or an isoleucine at position 24 of SEQ ID NO: 56;
c) a threonine at a position 21, a leucine at position 23, an alanine at position 25, a glycine at position 80, and/or a histidine at position 207 of SEQ ID NO: 58;
d) a threonine or a valine at position 15, and/or a tyrosine or a valine at position 203 of SEQ ID NO: 88;
e) a leucine at position 199 and/or a histidine at position 200 of SEQ ID NO: 92; and/or
f) a leucine at position 199, a histidine at position 200, an asparagine at position 201, and/or a histidine at position 202 of SEQ ID NO: 93.

Embodiment 161. The contiguous polypeptide of any one of embodiments 149 to 160, wherein the variant IgG Fc polypeptide comprises:
a) an amino acid substitution at a position corresponding to position 93 of SEQ ID NO: 54 or SEQ ID NO: 56;
b) an amino acid substitution at a position corresponding to position 87 of SEQ ID NO: 87, SEQ ID NO: 90, SEQ ID NO: 91, or SEQ ID NO: 94; or
c) an amino acid substitution at a position corresponding to position 198 of SEQ ID NO: 103, SEQ ID NO: 104, SEQ ID NO: 105, or SEQ ID NO: 106.

Embodiment 162. The contiguous polypeptide of any one of embodiments 149 to 161, wherein the variant IgG Fc polypeptide comprises:
a) an amino acid substitution at position 93 of SEQ ID NO: 54 or SEQ ID NO: 56;
b) an amino acid substitution at position 87 of SEQ ID NO: 87, SEQ ID NO: 90, SEQ ID NO: 91, or SEQ ID NO: 94; or
c) an amino acid substitution at position 198 of SEQ ID NO: 103, SEQ ID NO: 104, SEQ ID NO: 105, or SEQ ID NO: 106.

Embodiment 163. The contiguous polypeptide of any one of embodiments 149 to 162, wherein the variant IgG Fc polypeptide comprises:
a) an arginine at a position corresponding to position 93 of SEQ ID NO: 54 or SEQ ID NO: 56;
b) a serine at a position corresponding to position 87 of SEQ ID NO: 87, SEQ ID NO: 90, SEQ ID NO: 91, or SEQ ID NO: 94; or
c) an alanine at a position corresponding to position 198 of SEQ ID NO: 103, SEQ ID NO: 104, SEQ ID NO: 105, or SEQ ID NO: 106.

Embodiment 164. The contiguous polypeptide of any one of embodiments 149 to 163, wherein the variant IgG Fc polypeptide comprises:
a) an arginine at position 93 of SEQ ID NO: 54 or SEQ ID NO: 56;
b) a serine at position 87 of SEQ ID NO: 87, SEQ ID NO: 90, SEQ ID NO: 91, or SEQ ID NO: 94; or
c) an alanine at position 198 of SEQ ID NO: 103, SEQ ID NO: 104, SEQ ID NO: 105, or SEQ ID NO: 106.

Embodiment 165. The contiguous polypeptide of any one of embodiments 149 to 164, wherein the variant IgG Fc polypeptide comprises:
a) an amino acid substitution at a position corresponding to position 5, position 38, position 39, position 97, and/or position 98 of SEQ ID NO: 54; or
b) an amino acid substitution at a position corresponding to position 5, position 38, position 39, position 97, and/or position 98 of SEQ ID NO: 56.

Embodiment 166. The contiguous polypeptide of any one of embodiments 149 to 165, wherein the variant IgG Fc polypeptide comprises:
a) an amino acid substitution at position 5, position 38, position 39, position 97, and/or position 98 of SEQ ID NO: 54; or
b) an amino acid substitution at position 5, position 38, position 39, position 97, and/or position 98 of SEQ ID NO: 56.

Embodiment 167. The contiguous polypeptide of any one of embodiments 149 to 166, wherein the variant IgG Fc polypeptide comprises:
a) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 54; or
b) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 56.

Embodiment 168. The contiguous polypeptide of any one of embodiments 149 to 167, wherein the variant IgG Fc polypeptide comprises:
a) a proline at position 5, a glycine at position 38, an arginine at position 39, an isoleucine at position 97, and/or a glycine at position 98 of SEQ ID NO: 54; or
b) a proline at position 5, a glycine at position 38, an arginine at position 39, an isoleucine at position 97, and/or a glycine at position 98 of SEQ ID NO: 56.

Embodiment 169. The contiguous polypeptide of any one of embodiments 149 to 168, wherein the variant IgG Fc polypeptide comprises the amino acid sequence of SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, or SEQ ID NO: 111.

Embodiment 170. An isolated nucleic acid encoding the polypeptide of any one of embodiments 147 to 169.

Embodiment 171. A host cell comprising the nucleic acid of embodiment 170.

Embodiment 172. A method of producing a polypeptide comprising culturing the host cell of embodiment 171 and isolating the polypeptide.

Embodiment 173. A pharmaceutical composition comprising the polypeptide of any one of embodiments 147 to 169 and a pharmaceutically acceptable carrier.

Embodiment 174. A method of treating a companion animal having polycythemia, the method comprising administering to the subject a therapeutically effective amount of the polypeptide of any one of any one of embodiments 147 to 169, the nucleic acid of embodiment 170, or the pharmaceutical composition of embodiment 173.

Embodiment 175. The method of embodiment 174, wherein the polypeptide, nucleic acid, or pharmaceutical composition is administered parenterally.

Embodiment 176. The method of embodiment 174 or embodiment 175, wherein the polypeptide, nucleic acid, or pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 177. The method of any one of embodiments 174 to 176, wherein the companion animal species is feline, canine, or equine.

Embodiment 178. The method of any one of embodiments 174 to 177, wherein the polycythemia is caused by a mutation in JAK2, overproduction and/or secretion of EPO from a tumor.

These and other aspects and various embodiments are described more fully below.

### BRIEF DESCRIPTION OF THE DRAWINGS

FIG. 1A and FIG. 1B show Western blots of transient expression using 293 cells of different canine EPO polypeptide analogs having either additional N-glycosylation site(s) or additional intramolecular disulfide. Lane M: marker; Lane 1: wild-type canine EPO polypeptide (SEQ ID NO: 2); Lanes 2-12, 14: canine EPO polypeptide analogs A-L (SEQ ID NOs: 10, 12, 14, 16, 18, 20, 22, 24, 26, 28, 30, and 32, respectively); Lane 13: canine EPO-canine Fc fusion.

### DESCRIPTION OF CERTAIN SEQUENCES

Table 1 provides a listing of certain sequences referenced herein.

TABLE 1
Description of Certain Sequences

| SEQ | | |
|---|---|---|
| ID | | |
| NO: | SEQUENCE | DESCRIPTION |
| 1 | MGACECPALFLLLSLLLLPLGLPVLGAPPRLICDSRVLE | Canis lupus EPO |
| | RYILEAREAENVTMGCAQGCSFSENITVPDTKVNFYTWK | precursor form |
| | RMDVGQQALEVWQGLALLSEAILRGQALLANASQPSETP | |
| | QLHVDKAVSSLRSLTSLLRALGAQKEAMSLPEEASPAPL | |
| | RTFTVDTLCKLFRIYSNFLRGKLTLYTGEACRRGDR | |
| 2 | APPRLICDSRVLERYILEAREAENVTMGCAQGCSFSENI | Canis lupus EPO |
| | TVPDTKVNFYTWKRMDVGQQALEVWQGLALLSEAILRGQ | mature form |
| | ALLANASQPSETPQLHVDKAVSSLRSLTSLLRALGAQKE | |
| | AMSLPEEASPAPLRTFTVDTLCKLFRIYSNFLRGKLTLY | |
| | TGEACRRGDR | |
| 3 | MGVRECPALLLLLSLLLPPLGLPALGAPPRLICDSRVLE | Equus caballus EPO |
| | RYILEAREAENVTMGCAEGCSFGENVTVPDTKVNFYSWK | precursor form |
| | RMEVEQQAVEVWQGLALLSEAILQGQALLANSSQPSETL | |
| | RLHVDKAVSSLRSLTSLLRALGAQKEAISPPDAASAAPL | |
| | RTFAVDTLCKLFRIYSNFLRGKLKLYTGEACRRGDR | |
| 4 | APPRLICDSRVLERYILEAREAENVTMGCAEGCSFGENV | Equus caballus EPO |
| | TVPDTKVNFYSWKRMEVEQQAVEVWQGLALLSEAILQGQ | mature form |
| | ALLANSSQPSETLRLHVDKAVSSLRSLTSLLRALGAQKE | |
| | AISPPDAASAAPLRTFAVDTLCKLFRIYSNFLRGKLKLY | |
| | TGEACRRGDR | |
| 5 | MGSCECPALLLLLSLLLLPLGLPVLGAPPRLICDSRVLE | Felis catus |
| | RYILGAREAENVTMGCAEGCSFSENITVPDTKVNFYTWK | EPO precursor form |
| | RMDVGQQAVEVWQGLALLSEATLRGQALLANSSQPSETL | “wild-type feline |
| | QLHVDKAVSSLRSLTSLLRALGAQKEATSLPEATSAAPL | EPO G44” |
| | RTFTVDTLCKLFRIYSNFLRGKLTLYTGEACRRGDR | |
| 6 | APPRLICDSRVLERYILGAREAENVTMGCAEGCSFSENI | Felis catus |
| | TVPDTKVNFYTWKRMDVGQQAVEVWQGLALLSEAILRGQ | EPO mature form |
| | ALLANSSQPSETLQLHVDKAVSSLRSLTSLLRALGAQKE | “wild-type feline |
| | ATSLPEATSAAPLRTFTVDTLCKLFRIYSNFLRGKLTLY | EPO G18” |
| | TGEACRRGDR | |
| 7 | MGSCECPALLLLLSLLLLPLGLPVLGAPPRLICDSRVLE | Felis catus |
| | RYILEAREAENVTMGCAEGCSFSENITVPDTKVNFYTWK | EPO precursor form |
| | RMDVGQQAVEVWQGLALLSEAILRGQALLANSSQPSETL | “wild-type feline |
| | QLHVDKAVSSLRSLTSLLRALGAQKEATSLPEATSAAPL | EPO E44” |
| | RTFTVDTLCKLFRIYSNFLRGKLTLYTGEACRRGDR | |
| 8 | APPRLICDSRVLERYILEAREAENVTMGCAEGCSFSENI | Felis catus |
| | TVPDTKVNFYTWKRMDVGQQAVEVWQGLALLSEAILRGQ | EPO mature form |
| | ALLANSSQPSETLQLHVDKAVSSLRSLTSLLRALGAQKE | “wild-type feline |
| | ATSLPEATSAAPLRTFTVDTLCKLFRIYSNFLRGKLTLY | EPO E18” |
| | TGEACRRGDR | |
| 9 | MGACECPALFLLLSLLLLPLGLPVLGAPPRLICDSRVLE | Canine EPO analog |
| | RYILEAREAENVTMGCNQTCSFSENITVPDTKVNFYTWK | A precursor |
| | RMDVGQQALEVWQGLALLSEAILRGQALLANASQENETP | A56N |
| | QLHVDKAVSSLRSLTSLLRALGAQKEAMSLPEEASPAPL | G58T |
| | RTFTVDTLCKLFRIYSNFLRGKLTLYTGEACRRGDR | P113E |
| | | S114N |
| 10 | APPRLICDSRVLERYILEAREAENVTMGCNQTCSFSENI | Canine EPO analog |
| | TVPDTKVNFYTWKRMDVGQQALEVWQGLALLSEAILRGQ | A mature |
| | ALLANASQENETPQLHVDKAVSSLRSLTSLLRALGAQKE | A30N |
| | AMSLPEEASPAPLRTFTVDTLCKLFRIYSNFLRGKLTLY | G32T |
| | TGEACRRGDR | P87E |
| | | S88N |
| 11 | MGACECPALFLLLSLLLLPLGLPVLGAPPRLICDSRVLE | Canine EPO analog |
| | RYILEAREAENVTMGCNQTCSFSENITVPDTKVNFYTWK | B precursor A56N |
| | RMDVGQQALEVWQGLALLSEAILRGQALLANASQVNETP | G58T |
| | QLHVDKAVSSLRSLTSLLRALGAQKEAMSLPEEASPAPL | P113V |
| | RTFTVDTLCKLFRIYSNFLRGKLTLYTGEACRRGDR | S114N |
| 12 | APPRLICDSRVLERYILEAREAENVTMGCNQTCSFSENI | Canine EPO analog |
| | TVPDTKVNFYTWKRMDVGQQALEVWQGLALLSEAILRGQ | B mature |
| | ALLANASQVNETPQLHVDKAVSSLRSLTSLLRALGAQKE | A30N |
| | AMSLPEEASPAPLRTFTVDTLCKLFRIYSNFLRGKLTLY | G32T |
| | TGEACRRGDR | P87V |
| | | S88N |
| 13 | MGACECPALFLLLSLLLLPLGLPVLGAPPRLICDSRVLE | Canine EPO analog |
| | RYILEAREAENVTMGCAQGCSFSENITVPDTKVNFYTWK | C precursor |
| | RMNVTQQALEVWQGLALLSEAILRGQALLANASQPSETP | D81N |
| | QLHVDKAVSSLRSLTSLLRALGAQKEAMSLPEEASPAPL | G83T |
| | RTFTVDTLCKLFRIYSNFLRGKLTLYTGEACRRGDR | |
| 14 | APPRLICDSRVLERYILEAREAENVTMGCAQGCSFSENI | Canine EPO analog |
| | TVPDTKVNFYTWKRMNVTQQALEVWQGLALLSEAILRGQ | C mature |
| | ALLANASQPSETPQLHVDKAVSSLRSLTSLLRALGAQKE | D55N |
| | AMSLPEEASPAPLRTFTVDTLCKLFRIYSNFLRGKLTLY | G57T |
| | TGEACRRGDR | |
| 15 | MGACECPALFLLLSLLLLPLGLPVLGAPPRLICDSRVLE | Canine EPO analog |
| | RYILEAREAENVTMGCAQGCSFSENITVPDTKVNFYTWK | D precursor |
| | RMDVGQQALEVWQGLALLSEAILRGQALLANASQPSETP | L138N |
| | QLHVDKAVSSLRSLTSLLRANGTQKEAMSLPEEASPAPL | A140T |
| | RTFTVDTLCKLFRIYSNFLRGKLTLYTGEACRRGDR | |
| 16 | APPRLICDSRVLERYILEAREAENVTMGCAQGCSFSENI | Canine EPO analog |
| | TVPDTKVNFYTWKRMDVGQQALEVWQGLALLSEAILRGQ | D mature |
| | ALLANASQPSETPQLHVDKAVSSLRSLTSLLRANGTQKE | L112N |
| | AMSLPEEASPAPLRTFTVDTLCKLFRIYSNFLRGKLTLY | A114T |
| | TGEACRRGDR | |
| 17 | MGACECPALFLLLSLLLLPLGLPVLGAPPRLICDSRVLE | Canine EPO analog |
| | RYILEAREAENVTMGCAQGCSFSENITVPDTKVNFYTWK | E precursor |
| | RMDVGQQALEVWQGLALLSEAILRGQALLANASQPSETP | L147N |
| | QLHVDKAVSSLRSLTSLLRALGAQKEAMSNVTEASPAPL | P148V |
| | RTFTVDTLCKLFRIYSNFLRGKLTLYTGEACRRGDR | E149T |
| 18 | APPRLICDSRVLERYILEAREAENVTMGCAQGCSFSENI | Canine EPO analog |
| | TVPDTKVNFYTWKRMDVGQQALEVWQGLALLSEAILRGQ | E mature |
| | ALLANASQPSETPQLHVDKAVSSLRSLTSLLRALGAQKE | L121N |
| | AMSNVTEASPAPLRTFTVDTLCKLFRIYSNFLRGKLTLY | P122V |
| | TGEACRRGDR | E123T |
| 19 | MGACECPALFLLLSLLLLPLGLPVLGAPPRLICDSRVLE | Canine EPO analog |
| | RYILEAREAENVTMGCAQGCSFSENITVPDTKVNFYTWK | F precursor |
| | RMDVGQQALEVWQGLALLSEAILRGQALLANASQPSETP | P148E |
| | QLHVDKAVSSLRSLTSLLRALGAQKEAMSLENETSPAPL | E149N |
| | RTFTVDTLCKLFRIYSNFLRGKLTLYTGEACRRGDR | A151T |
| 20 | APPRLICDSRVLERYILEAREAENVTMGCAQGCSFSENI | Canine EPO analog |
| | TVPDTKVNFYTWKRMDVGQQALEVWQGLALLSEAILRGQ | F mature |
| | ALLANASQPSETPQLHVDKAVSSLRSLTSLLRALGAQKE | P122E |
| | AMSLENETSPAPLRTFTVDTLCKLFRIYSNFLRGKLTLY | E123N |
| | TGEACRRGDR | A125T |
| 21 | MGACECPALFLLLSLLLLPLGLPVLGAPPRLICDSRVLE | Canine EPO analog |
| | RYILECREAENVTMGCAQGCSFSENITVPDTKVNFYTWK | G precursor |
| | RMDVGQQALEVWQGLALLSEAILRGQALLANASQPSETP | A45C |
| | QLHVDKAVSSLRSLTSLLRALGAQKEAMSLPEEASPAPL | S172C |
| | RTFTVDTLCKLFRIYCNFLRGKLTLYTGEACRRGDR | |
| 22 | APPRLICDSRVLERYILECREAENVTMGCAQGCSFSENI | Canine EPO analog |
| | TVPDTKVNFYTWKRMDVGQQALEVWQGLALLSEAILRGQ | G mature |
| | ALLANASQPSETPQLHVDKAVSSLRSLTSLLRALGAQKE | A19C |
| | AMSLPEEASPAPLRTFTVDTLCKLFRIYCNFLRGKLTLY | S146C |
| | TGEACRRGDR | |
| 23 | MGACECPALFLLLSLLLLPLGLPVLGAPPRLICDSRVLE | Canine EPO analog |
| | RYILEARECENVTMGCAQGCSFSENITVPDTKVNFYTWK | H precursor |
| | RMDVGQQALEVWQGLALLSEAILRGQALLANASQPSETP | A48C |
| | QLCVDEAVSSLRSLTSLLRALGAQKEAMSLPEEASPAPL | H120C |
| | RTFTVDTLCKLFRIYSNFLRGKLTLYTGEACRRGDR | |
| 24 | APPRLICDSRVLERYILEARECENVTMGCAQGCSFSENI | Canine EPO analog |
| | TVPDTKVNFYTWKRMDVGQQALEVWQGLALLSEAILRGQ | H mature |
| | ALLANASQPSETPQLCVDKAVSSLRSLTSLLRALGAQKE | A22C |
| | AMSLPEEASPAPLRTFTVDTLCKLFRIYSNFLRGKLTLY | H94C |
| | TGEACRRGDR | |
| 25 | MGACECPALFLLLSLLLLPLGLPVLGAPPRLICDSRVLE | Canine EPO analog I |
| | RYILEAREACNVTMGCAQGCSFSENITVPDTKVNFYTWK | precursor |
| | RMDVGQQALEVWQGLALLSEAILRGQALLANASQPSETP | E49C |
| | QLHVDKAVSSLRSLTSLLRALGAQKEAMSLPEEASPAPL | S172C |
| | RTFTVDTLCKLFRIYCNFLRGKLTLYTGEACRRGDR | |
| 26 | APPRLICDSRVLERYILEAREACNVTMGCAQGCSFSENI | Canine EPO analog I |
| | TVPDTKVNFYTWKRMDVGQQALEVWQGLALLSEAILRGQ | mature |
| | ALLANASQPSETPQLHVDKAVSSLRSLTSLLRALGAQKE | E23C |
| | AMSLPEEASPAPLRTFTVDTLCKLFRIYCNFLRGKLTLY | S146C |
| | TGEACRRGDR | |
| 27 | MGACECPALFLLLSLLLLPLGLPVLGAPPRLICDSRVLE | Canine EPO analog J |
| | RYILEAREAENVTMGCAQGCSFSENITVCDTKVNFYTWK | precursor |
| | RMDVGQQALEVWQCLALLSEAILRGQALLANASQPSETP | P68C |
| | QLHVDKAVSSLRSLTSLLRALGAQKEAMSLPEEASPAPL | G92C |
| | RTFTVDTLCKLFRIYSNFLRGKLTLYTGEACRRGDR | |
| 28 | APPRLICDSRVLERYILEAREAENVTMGCAQGCSFSENI | Canine EPO analog J |
| | TVCDTKVNFYTWKRMDVGQQALEVWQCLALLSEAILRGQ | mature |
| | ALLANASQPSETPQLHVDKAVSSLRSLTSLLRALGAQKE | P42C |
| | AMSLPEEASPAPLRTFTVDTLCKLFRIYSNFLRGKLTLY | G66C |
| | TGEACRRGDR | |
| 29 | MGACECPALFLLLSLLLLPLGLPVLGAPPRLICDSRVLE | Canine EPO analog |
| | RYILEAREAENVTMGCAQGCSFSENITVPDTKVNFYTWK | K precursor |
| | RMDVGQQALEVCQGLALLSEAILRGQALLANASQPSETP | W90C |
| | QLHVDKAVSSLRSLTSLLRALGAQKECMSLPEEASPAPL | A144C |
| | RTFTVDTLCKLFRIYSNFLRGKLTLYTGEACRRGDR | |
| 30 | APPRLICDSRVLERYILEAREAENVTMGCAQGCSFSENI | Canine EPO analog |
| | TVPDTKVNFYTWKRMDVGQQALEVCQGLALLSEAILRGQ | K mature |
| | ALLANASQPSETPQLHVDKAVSSLRSLTSLLRALGAQKE | W64C |
| | CMSLPEEASPAPLRTFTVDTLCKLFRIYSNFLRGKLTLY | A118C |
| | TGEACRRGDR | |
| 31 | MGACECPALFLLLSLLLLPLGLPVLGAPPRLICDSRVLE | Canine EPO analog |
| | RYILEAREAENVTMGCAQGCSFSENITVPDTKVNFYTWK | L precursor |
| | RMDVGQQCLEVWQGLALLSEAILRGQALLANASQPSETP | A86C |
| | QLHVDKAVSSLRSLTSLLRALGAQKCAMSLPEEASPAPL | E143C |
| | RTFTVDTLCKLFRIYSNFLRGKLTLYTGEACRRGDR | |
| 32 | APPRLICDSRVLERYILEARFAENVTMGCAQGCSFSENI | Canine EPO analog |
| | TVPDTKVNFYTWKRMDVGQQCLEVWQGLALLSEAILRGQ | L mature |
| | ALLANASQPSETPQLHVDKAVSSLRSLTSLLRALGAQKC | A60C |
| | AMSLPEEASPAPLRTFTVDTLCKLFRIYSNFLRGKLTLY | E117C |
| | TGEACRRGDR | |
| 33 | MNHLWTHLWPGVGSLCLLLAGAAWASLPKPLDPKFESKA | Canis lupus EPO |
| | ALLAARAPEELLCFTERLEDLVCFWEEAASAGVGPDNYS | receptor precursor |
| | FFYQLEGEPWKTCSLHQAPTTRGAVRFWCSLPTADTSSF | form |
| | VPLELRATAVSSGALLYRRIIHINEVVLLDPPAGLLARR | |
| | ADEGGHVVLRWLPPPGAPVASLIRYEVNISGSVAGGSQK | |
| | VEILDGRTECVLSNLRGGTRYTFMVRARMAEPSFGGFWS | |
| | AWSEPASLLTASDLDPLILTLSLILVLILLLLAVLALLS | |
| | HRRTLKQKIWPGIPSPESEFEGLFTTHKGNFQLWLYQNE | |
| | GCLWWSPCTPLAEDPPAPLEVLSERCWGAPQAVEPGADD | |
| | EGPLLEPVGSEHSQDTYLVLDKWLLPRNPSSEDVSQSGG | |
| | SLDIVAMDKGSEASSCSSGLSLKPGPEGALGASFEYTIL | |
| | DPSSQLLCPRALPPELPPTPPHIKYLYLMVSDSGISTDY | |
| | SSGGSQGAQGDSLNSPFLNPYENSLIPAPEPSPPGYVAC | |
| | S | |
| 34 | MNHLWTHLWPGVGSLCLLLAGAAWASLPKPLDPKFESKA | Exemplary canine |
| | ALLAARAPEELLCFTERLEDLVCFWEEAASAGVGPDNYS | EPOR ECD |
| | FFYQLEGEPWKTCSLHQAPTTRGAVRFWCSLPTADTSSF | |
| | VPLELRATAVSSGALLYRRIIHINEVVLLDPPAGLLARR | |
| | ADEGGHVVLRWLPPPGAPVASLIRYEVNISGSVAGGSQK | |
| | VEILDGRTECVLSNLRGGTRYTFMVRARMAEPSFGGFWS | |
| | AWSEPASLLTASDLD | |
| 35 | DPKFESKAALLAARAPEELLCFTERLEDLVCFWEEAASA | Exemplary canine |
| | GVGPDNYSFFYQLEGEPWKTCSLHQAPTTRGAVRFWCSL | EPOR minimal ECD |
| | PTADTSSFVPLELRATAVSSGALLYRRIIHINEVVLLDP | |
| | PAGLLARRADEGGHVVLRWLPPPGAPVASLIRYEVNISG | |
| | SVAGGSQKVEILDGRTECVLSNLRGGTRYTFMVRARMAE | |
| | PSFGGFWSAWSEPASLLT | |
| 36 | MNHLWTHLWPGVGSLCLLLAGAAWASLPKPLDPKFESKA | Exemplary canine |
| | ALLAARAPEELLCFTERLEDLVCFWEEAASAGVGPDNYS | EPOR ECD - |
| | FFYQLEGEPWKTCSLHQAPTTRGAVRFWCSLPTADTSSF | Human Fc |
| | VPLELRATAVSSGALLYRRIIHINEVVLLDPPAGLLARR | |
| | ADEGGHVVLRWLPPPGAPVASLIRYEVNISGSVAGGSQK | |
| | VEILDGRTECVLSNLRGGTRYTFMVRARMAEPSFGGFWS | |
| | AWSEPASLLTASDLDIEGRMDPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPGK | |
| 37 | MNHLGAPLWPGVGSLCLLLAGAAWAPPPNSSDPRFESKA | Equus caballus EPO |
| | ALLAARGPEELLCFTERLEDLVCFWEEAASAGVGPENYS | receptor precursor |
| | FSYQLEGEPWKPCRLHQASTARGAVRFWCSLPTADTSSF | form |
| | VPLELRVTAATSGAPRYRRVIQVNEVVLLDPPAGLLARL | |
| | ADEGGHVLLRWLPPPGAPMASLIRYEVNISAGNAAGGAQ | |
| | RVEILDGRTECVLSNLRGQTRYTFAVRARMAEPSFGGFW | |
| | SAWSEPASLLTASDLDPLLLTLSLILVLILLLLAVLALL | |
| | SHRRALKQKIWPGIPSPESEFEGLFTTHKGNFQLWLYQN | |
| | DGCLWWNPCTPFTEDPPASLEVLSERCWGVTQAVEPGAE | |
| | DEGPLLEPVGSEHARDPYLVLDKWLLPRSPTSEDLPQPG | |
| | GGLDTAAMDAGSEASSCSSALALKPGPEGASAASFEYTI | |
| | LDPSSQLLRPRALPPELPPTPPHLKYLYLVVSDSGISTD | |
| | YSSGGSQGAQRGSSDGPYSNPYENSLVPAPEPSAPSYVA | |
| | CS | |
| 38 | MNHLGAPLWPGVGSLCLLLAGAAWAPPPNSSDPRFESKA | Exemplary equine |
| | ALLAARGPEELLCFTERLEDLVCFWEEAASAGVGPENYS | EPOR ECD |
| | FSYQLEGEPWKPCRLHQASTARGAVRFWCSLPTADTSSF | |
| | VPLELRVTAATSGAPRYRRVIQVNEVVLLDPPAGLLARL | |
| | ADEGGHVLLRWLPPPGAPMASLIRYEVNISAGNAAGGAQ | |
| | RVEILDGRTECVLSNLRGQTRYTFAVRARMAEPSFGGFW | |
| | SAWSEPASLLTASDLD | |
| 39 | DPRFESKAALLAARGPEELLCFTERLEDLVCFWEEAASA | Exemplary equine |
| | GVGPENYSFSYQLEGEPWKPCRLHQASTARGAVRFWCSL | EPOR minimal ECD |
| | PTADTSSFVPLELRVTAATSGAPRYRRVIQVNEVVLLDP | |
| | PAGLLARLADEGGHVLLRWLPPPGAPMASLIRYEVNISA | |
| | GNAAGGAQRVEILDGRTECVLSNLRGQTRYTFAVRARMA | |
| | EPSFGGFWSAWSEPASLLT | |
| 40 | MNHLGAPLWPGVGSLCLLLAGAAWAPPPNSSDPRFESKA | Exemplary equine |
| | ALLAARGPEELLCFTERLEDLVCFWEEAASAGVGPENYS | EPOR ECD - |
| | FSYQLEGEPWKPCRLHQASTARGAVRFWCSLPTADTSSF | Human Fc |
| | VPLELRVTAATSGAPRYRRVIQVNEVVLLDPPAGLLARL | |
| | ADEGGHVLLRWLPPPGAPMASLIRYEVNISAGNAAGGAQ | |
| | RVEILDGRTECVLSNLRGQTRYTFAVRARMAEPSFGGFW | |
| | SAWSEPASLLTASDLDIEGRMDPKSCDKTHTCPPCPAPE | |
| | LLGGPSVFLFPPKPKDTLMISRTPEVTCVVVDVSHEDPE | |
| | VKFNWYVDGVEVHNAKTKPREEQYNSTYRVVSVLTVLHQ | |
| | DWLNGKEYKCKVSNKALPAPIEKTISKAKGQPREPQVYT | |
| | LPPSRDELTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPGK | |
| 41 | MDHLWAPLWPGVGSLCLLLAGAAWAPPPNPLDPKFESKV | Felis catus |
| | NMVCMRAPEASACGSSERLEDLVCFWEEAASAGVGPDNY | EPO receptor |
| | SFFYQLEGEPWKPCSLHQAPTARGAVRFWCSLPTADASS | Sequence EPOR201 |
| | FVPLELRVTAVSSGAPRYHRIIHINEVVLLDPPAGLLAR | UniProtKB - |
| | RADEGGHVVLRWLPPPGAPVASLIRYEVNISSGNVAGGA | M3X491 |
| | QKVEILDGRTECALSNLRGRTRYTFMVRARMAEPSFGGF | |
| | WSAWSEPASLLTASDLDPLILTLSLILVLILLLLAVLAL | |
| | LSHRRFTRTLKQKIWPGIPSPESEFEGLFTTHKGNFQLW | |
| | LYQNEGCLWWSPCAPFAEDPPSPLEVLSERCWGATQAAE | |
| | PGAEEGPLLEPLGSEHTQDTYLVLDKWLLPRNPPSEDLP | |
| | RPDGSLDMVAMHKGSEASSCSSALSLKPGPEGALGASFE | |
| | YTILDPSSQLLRPRALPPELPPTPPHIKYLYLMVSDSGI | |
| | STDYSSGGSQEAQGDSSTGPYLNPYENSLIPATETSPPS | |
| | YVACS | |
| 42 | PPPNPLDPKFESKVNMVCMRAPEASACGSSERLEDLVCF | Exemplary Feline |
| | WEEAASAGVGPDNYSFFYQLEGEPWKPCSLHQAPTARGA | EPOR201 ECD |
| | VRFWCSLPTADASSFVPLELRVTAVSSGAPRYHRIIHIN | |
| | EVVLLDPPAGLLARRADEGGHVVLRWLPPPGAPVASLIR | |
| | YEVNISSGNVAGGAQKVEILDGRTECALSNLRGRTRYTF | |
| | MVRARMAEPSFGGFWSAWSEPASLLTASDLD | |
| 43 | DPKFESKVNMVCMRAPEASACGSSERLEDLVCFWEEAAS | Exemplary Feline |
| | AGVGPDNYSFFYQLEGEPWKPCSLHQAPTARGAVRFWCS | EPOR201 minimal |
| | LPTADASSFVPLELRVTAVSSGAPRYHRIIHINEVVLLD | ECD |
| | PPAGLLARRADEGGHVVLRWLPPPGAPVASLIRYEVNIS | |
| | SGNVAGGAQKVEILDGRTECALSNLRGRTRYTFMVRARM | |
| | AEPSFGGFWSAWSEPASLLT | |
| 44 | MDHLWAPLWPGVGSLCLLLAGAAWAPPPNPLDPKFESKG | Felis catus |
| | KDGSVCRPPQWFLEGNAEERLEDLVCFWEEAASAGVGPD | EPO receptor |
| | NYSFFYQLEGEPWKPCSLHQAPTARGAVRFWCSLPTADA | Sequence EPOR202 |
| | SSFVPLELRVTAVSSGAPRYHRIIHINEVVLLDPPAGLL | |
| | ARRADEGGHVVLRWLPPPGAPVASLIRYEVNISSGNVAG | |
| | GAQKVEILDGRTECALSNLRGRTRYTFMVRARMAEPSFG | |
| | GFWSAWSEPASLLTASDLDPLILTLSLILVLILLLLAVL | |
| | ALLSHRRTLKQKIWPGIPSPESEFEGLFTTHKGNFQLWL | |
| | YQNEGCLWWSPCAPFAEDPPSPLEVLSERCWGATQAAEP | |
| | GAEEGPLLEPLGSEHTQDTYLVLDKWLLPRNPPSEDLPR | |
| | PDGSLDMVAMHKGSEASSCSSALSLKPGPEGALGASFEY | |
| | TILDPSSQLLRPRALPPELPPTPPHIKYLYLMVSDSGIS | |
| | TDYSSGGSQEAQGDSSTGPYLNPYENSLIPATETSPPSY | |
| | VACS | |
| 45 | PPPNPLDPKFESKGKDGSVCRPPQWFLEGNAEERLEDLV | Exemplary feline |
| | CFWEEAASAGVGPDNYSFFYQLEGEPWKPCSLHQAPTAR | EPOR202 ECD |
| | GAVRFWCSLPTADASSFVPLELRVTAVSSGAPRYHRIIH | |
| | INEVVLLDPPAGLLARRADEGGHVVLRWLPPPGAPVASL | |
| | IRYEVNISSGNVAGGAQKVEILDGRTECALSNLRGRTRY | |
| | TFMVRARMAEPSFGGFWSAWSEPASLLTASDLD | |
| 46 | DPKFESKGKDGSVCRPPQWFLEGNAEERLEDLVCFWEEA | Exemplary feline |
| | ASAGVGPDNYSFFYQLEGEPWKPCSLHQAPTARGAVRFW | EPOR202 minimal |
| | CSLPTADASSFVPLELRVTAVSSGAPRYHRIIHINEVVL | ECD |
| | LDPPAGLLARRADEGGHVVLRWLPPPGAPVASLIRYEVN | |
| | ISSGNVAGGAQKVEILDGRTECALSNLRGRTRYTFMVRA | |
| | RMAEPSFGGFWSAWSEPASLLT | |
| 47 | MDHLWAPLWPGVGSLCLLLAGAAWAPPPNPLDPKFESKX | Felis catus |
| | ALLAARGPEELLCFTERLEDLVCFWEEAASAGVGPDNYS | EPO receptor |
| | FFYQLEGEPWKPCSLHQAPTARGAVRFWCSLPTADASSF | Sequence EPOR203 |
| | VPLELRVTAVSSGAPRYHRIIHINEVVLLDPPAGLLARR | NCBI Reference |
| | ADEGGHVVLRWLPPPGAPVASLIRYEVNISSGNVAGGAQ | Sequence: |
| | KVEILDGRTECALSNLRGRTRYTFMVRARMAEPSFGGFW | XP_019673378.1 |
| | SAWSEPASLLTASDLDPLILTLSLILVLILLLLAVLALL | |
| | SHRRTLKQKIWPGIPSPESEFEGLFTTHKGNFQLWLYQN | |
| | EGCLWWSPCAPFAEDPPSPLEVLSERCWGATQAAEPGAE | |
| | EGPLLEPLGSEHTQDTYLVLDKWLLPRNPPSEDLPRPDG | |
| | SLDMVAMHKGSEASSCSSALSLKPGPEGALGASFEYTIL | |
| | DPSSQLLRPRALPPELPPTPPHIKYLYLMVSDSGISTDY | |
| | SSGGSQEAQGDSSTGPYLNPYENSLIPATETSPPSYVAC | |
| | S | |
| 48 | PPPNPLDPKFESKXALLAARGPEELLCFTERLEDLVCFW | Exemplary feline |
| | EEAASAGVGPDNYSFFYQLEGEPWKPCSLHQAPTARGAV | EPOR203 ECD |
| | RFWCSLPTADASSFVPLELRVTAVSSGAPRYHRIIHINE | |
| | VVLLDPPAGLLARRADEGGHVVLRWLPPPGAPVASLIRY | |
| | EVNISSGNVAGGAQKVEILDGRTECALSNLRGRTRYTFM | |
| | VRARMAEPSFGGFWSAWSEPASLLTASDLDP | |
| 49 | DPKFESKXALLAARGPEELLCFTERLEDLVCFWEEAASA | Exemplary feline |
| | GVGPDNYSFFYQLEGEPWKPCSLHQAPTARGAVRFWCSL | EPOR203_39A |
| | PTADASSFVPLELRVTAVSSGAPRYHRIIHINEVVLLDP | minimal ECD |
| | PAGLLARRADEGGHVVLRWLPPPGAPVASLIRYEVNISS | |
| | GNVAGGAQKVEILDGRTECALSNLRGRTRYTFMVRARMA | |
| | EPSFGGFWSAWSEPASLLT | |
| 50 | MDHLWAPLWPGVGSLCLLLAGAAWAPPPNPLDPKFESKA | Exemplary feline |
| | ALLAARGPEELLCFTERLEDLVCFWEEAASAGVGPDNYS | EPO receptor |
| | FFYQLEGEPWKPCSLHQAPTARGAVRFWCSLPTADASSF | EPOR203_39A |
| | VPLELRVTAVSSGAPRYHRIIHINEVVLLDPPAGLLARR | |
| | ADEGGHVVLRWLPPPGAPVASLIRYEVNISSGNVAGGAQ | |
| | KVEILDGRTECALSNLRGRTRYTFMVRARMAEPSFGGFW | |
| | SAWSEPASLLTASDLDPLILTLSLILVLILLLLAVLALL | |
| | SHRRTLKQKIWPGIPSPESEFEGLFTTHKGNFQLWLYQN | |
| | EGCLWWSPCAPFAEDPPSPLEVLSERCWGATQAAEPGAE | |
| | EGPLLEPLGSEHTQDTYLVLDKWLLPRNPPSEDLPRPDG | |
| | SLDMVAMHKGSEASSCSSALSLKPGPEGALGASFEYTIL | |
| | DPSSQLLRPRALPPELPPTPPHIKYLYLMVSDSGISTDY | |
| | SSGGSQEAQGDSSTGPYLNPYENSLIPATETSPPSYVAC | |
| | S | |
| 51 | PPPNPLDPKFESKAALLAARGPEELLCFTERLEDLVCFW | Exemplary feline |
| | EEAASAGVGPDNYSFFYQLEGEPWKPCSLHQAPTARGAV | EPOR203_39A ECD |
| | RFWCSLPTADASSFVPLELRVTAVSSGAPRYHRIIHINE | |
| | VVLLDPPAGLLARRADEGGHVVLRWLPPPGAPVASLIRY | |
| | EVNISSGNVAGGAQKVEILDGRTECALSNLRGRTRYTFM | |
| | VRARMAEPSFGGFWSAWSEPASLLTASDLDP | |
| 52 | DPKFESKAALLAARGPEELLCFTERLEDLVCFWEEAASA | Exemplary feline |
| | GVGPDNYSFFYQLEGEPWKPCSLHQAPTARGAVRFWCSL | EPOR203_39A |
| | PTADASSFVPLELRVTAVSSGAPRYHRIIHINEVVLLDP | minimal ECD |
| | PAGLLARRADEGGHVVLRWLPPPGAPVASLIRYEVNISS | |
| | GNVAGGAQKVEILDGRTECALSNLRGRTRYTFMVRARMA | |
| | EPSFGGFWSAWSEPASLLT | |
| 53 | PVPEPLGGPSVLIFPPKPKDILRITRTPEVTCVVLDLGR | Exemplary wild-type |
| | EDPEVQISWFVDGKEVHTAKTQSREQQFNGTYRVVSVLP | canine IgG-A Fc |
| | IEHQDWLTGKEFKCRVNHIDLPSPIERTISKARGRAHKP | Protein A − |
| | SVYVLPPSPKELSSSDTVSITCLIKDFYPPDIDVEWQSN | C1q − |
| | GQQEPERKHRMTPPQLDEDGSYFLYSKLSVDKSRWQQGD | CD16 − |
| | PFTCAVMHETLQNHYTDLSLSHSPGK | |
| 54 | PAPEMLGGPSVFIFPPKPKDTLLIARTPEVTCVVVDLDP | Exemplary wild-type |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 + |
| | FICAVMHEALHNHYTQESLSHSPGK | |
| 55 | PKRENGRVPRPPDCPKCPAPEMLGGPSVFIFPPKPKDTL | Exemplary wild-type |
| | LIARTPEVTCVVVDLDPEDPEVQISWFVDGKQMQTAKTQ | canine IgG-B Fc |
| | PREEQFNGTYRVVSVLPIGHQDWLKGKQFTCKVNNKALP | with hinge |
| | SPIERTISKARGQAHQPSVYVLPPSREELSKNTVSLTCL | Protein A + |
| | IKDFFPPDIDVEWQSNGQQEPESKYRTTPPQLDEDGSYF | C1q + |
| | LYSKLSVDKSRWQRGDTFICAVMHEALHNHYTQESLSHS | CD16 + |
| | PGK | |
| 56 | PGCGLLGGPSVFIFPPKPKDILVTARTPTVTCVVVDLDP | Exemplary wild-type |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 + |
| | FICAVMHEALHNHYTQISLSHSPGK | |
| 57 | AKECECKCNCNNCPCPGCGLLGGPSVFIFPPKPKDILVT | Exemplary wild-type |
| | ARTPTVTCVVVDLDPENPEVQISWFVDSKQVQTANTQPR | canine IgG-C Fc |
| | EEQSNGTYRVVSVLPIGHQDWLSGKQFKCKVNNKALPSP | with hinge |
| | IEEIISKTPGQAHQPNVYVLPPSRDEMSKNTVTLTCLVK | Protein A − |
| | DFFPPEIDVEWQSNGQQEPESKYRMTPPQLDEDGSYFLY | C1q + |
| | SKLSVDKSRWQRGDTFICAVMHEALHNHYTQISLSHSPG | CD16 + |
| | K | |
| 58 | PVPESLGGPSVFIFPPKPKDILRITRTPEITCVVLDLGR | Exemplary wild-type |
| | EDPEVQISWFVDGKEVHTAKTQPREQQFNSTYRVVSVLP | canine IgG-D Fc |
| | IEHQDWLTGKEFKCRVNHIGLPSPIERTISKARGQAHQP | Protein A − |
| | SVYVLPPSPKELSSSDTVTLTCLIKDFFPPEIDVEWQSN | C1q − |
| | GQPEPESKYHTTAPQLDEDGSYFLYSKLSVDKSRWQQGD | CD16 − |
| | TFTCAVMHEALQNHYTDLSLSHSPGK | |
| 59 | PVPEPLGGPSVLIFPPKPKDTLLIARTPEVTCVVLDLGR | Exemplary variant |
| | EDPEVQISWFVDGKEVHTAKTQSREQQFNGTYRVVSVLP | canine IgG-A Fc |
| | IGHQDWLTGKEFKCRVNHIDLPSPIERTISKARGRAHKP | C1q − |
| | SVYVLPPSPKELSSSDTVSITCLIKDFYPPDIDVEWQSN | Protein A + |
| | GQQEPERKHRMTPPQLDEDGSYFLYSKLSVDKSRWQQGD | I(21)T |
| | PFTCAVMHEALHNHYTDLSLSHSPGK | R(23)L |
| | | T(25)A |
| | | E(80)G |
| | | T(205)A |
| | | Q(207)H |
| 60 | PGCGLLGGPSVFIFPPKPKDTLLIARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | C1q + |
| | NVYVLPPSRDEMSKNTVTLTCLVKDFFPPEIDVEWQSNG | Protein A + |
| | QQEPESKYRMTPPQLDEDGSYFLYSKLSVDKSRWQRGDT | I(21)T |
| | FICAVMHEALHNHYTQISLSHSPGK | V(23)L |
| | | T(24)I |
| 61 | PVPESLGGPSVFIFPPKPKDTLLIARTPEITCVVLDLGR | Exemplary variant |
| | EDPEVQISWFVDGKEVHTAKTQPREQQFNSTYRVVSVLP | canine IgG-D Fc |
| | IGHQDWLTGKEFKCRVNHIGLPSPIERTISKARGQAHQP | C1q − |
| | SVYVLPPSPKELSSSDTVTLTCLIKDFFPPEIDVEWQSN | Protein A + |
| | GQPEPESKYHTTAPQLDEDGSYFLYSKLSVDKSRWQQGD | I(21)T |
| | TFTCAVMHEALHNHYTDLSLSHSPGK | R(23)L |
| | | T(25)A |
| | | E(80)G |
| | | Q(207)H |
| 62 | PVPEPLGGPSVLIFPPKPKDTLRITRTPEVTCVVLDLGR | Exemplary variant |
| | EDPEVQISWFVDGKEVHTAKTQSREQQFNGTYRVVSVLP | canine IgG-A Fc |
| | IEHQDWLTGKEFKCRVNHIDLPSPIERTISKARGRAHKP | C1q − |
| | SVYVLPPSPKELSSSDTVSITCLIKDFYPPDIDVEWQSN | Protein A + |
| | GQQEPERKHRMTPPQLDEDGSYFLYSKLSVDKSRWQQGD | I(21)T |
| | PFTCAVMHETLHNHYTDLSLSHSPGK | Q(207)H |
| 63 | PGCGLLGGPSVFIFPPKPKDTLVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | C1q + |
| | NVYVLPPSRDEMSKNTVTLTCLVKDFFPPEIDVEWQSNG | Protein A + |
| | QQEPESKYRMTPPQLDEDGSYFLYSKLSVDKSRWQRGDT | I(21)T |
| | FICAVMHEALHNHYTQISLSHSPGK | |
| 64 | PVPESLGGPSVFIFPPKPKDTLRITRTPEITCVVLDLGR | Exemplary variant |
| | EDPEVQISWFVDGKEVHTAKTQPREQQFNSTYRVVSVLP | canine IgG-D Fc |
| | IEHQDWLTGKEFKCRVNHIGLPSPIERTISKARGQAHQP | C1q − |
| | SVYVLPPSPKELSSSDTVTLTCLIKDFFPPEIDVEWQSN | Protein A + |
| | GQPEPESKYHTTAPQLDEDGSYFLYSKLSVDKSRWQQGD | I(21)T |
| | TFTCAVMHEALHNHYTDLSLSHSPGK | Q(207)H |
| 65 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCRVNNKALPSPIERTISKARGQAHQP | Protein A + |
| | SVYVLPPSREELSKNTVSLTCLIKDFFPPDIDVEWQSNG | C1q − |
| | QQEPESKYRTTPPQLDEDGSYFLYSKLSVDKSRWQRGDT | K(93)R |
| | FICAVMHEALHNHYTQESLSHSPGK | |
| 66 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCRVNNKALPSPIEEIISKTPGQAHQP | Protein A − |
| | NVYVLPPSRDEMSKNTVTLTCLVKDFFPPEIDVEWQSNG | C1q − |
| | QQEPESKYRMTPPQLDEDGSYFLYSKLSVDKSRWQRGDT | CD16 + |
| | FICAVMHEALHNHYTQISLSHSPGK | K(93)R |
| 67 | PAPEPLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | M(5)P |
| 68 | PAPEMLGGPSVFIFPPKPKDTLLIARTPEVTCVVVDLDR | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | P(39)R |
| 69 | PAPEMLGGPSVFIFPPKPKDTLLIARTPEVTCVVVDLGP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | D(38)G |
| 70 | PAPEMLGGPSVFIFPPKPKDTLLIARTPEVTCVVVDLGR | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | D(38)G |
| | | P(39)R |
| 71 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNIALPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | K(97)I |
| 72 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKGLPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | A(98)G |
| 73 | PAPEMLGGPSVFIFPPKPKDTLLIARTPEVTCVVVDLGP | Exemplary variant |
| | EDPEVQISWFVDGKQMQTAKTQPREEQFNGTYRVVSVLP | canine IgG-B Fc |
| | IGHQDWLKGKQFTCKVNNIGLPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | D(38)G |
| | | K(97)I |
| | | A(98)G |
| 74 | PAPEPLGGPSVFIFPPKPKDTLLIARTPEVTCVVVDLDR | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A + |
| | SVYVLPPSREELSKNTVSLTCLIKDFFPPDIDVEWQSNG | C1q + |
| | QQEPESKYRTTPPQLDEDGSYFLYSKLSVDKSRWQRGDT | CD16 − |
| | FICAVMHEALHNHYTQESLSHSPGK | M(5)P |
| | | P(39)R |
| 75 | PGCGPLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 − |
| | FICAVMHEALHNHYTQISLSHSPGK | L(5)P |
| 76 | PGCGLLGGPSVFIFPPKPKDILVTARTPTVTCVVVDLDR | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 − |
| | FICAVMHEALHNHYTQISLSHSPGK | P(39)R |
| 77 | PGCGLLGGPSVFIFPPKPKDILVTARTPTVTCVVVDLGP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 − |
| | FICAVMHEALHNHYTQISLSHSPGK | D(38)G |
| 78 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNIALPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 − |
| | FICAVMHEALHNHYTQISLSHSPGK | K(97)I |
| 79 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKGLPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 − |
| | FICAVMHEALHNHYTQISLSHSPGK | A(98)G |
| 80 | PGCGPLGGPSVFIFPPKPKDILVTARTPTVTCVVVDLDR | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 − |
| | FICAVMHEALHNHYTQISLSHSPGK | L(5)P |
| | | P(39)R |
| 81 | PGCGLLGGPSVFIFPPKPKDILVTARTPTVTCVVVDLGP | Exemplary variant |
| | ENPEVQISWFVDSKQVQTANTQPREEQSNGTYRVVSVLP | canine IgG-C Fc |
| | IGHQDWLSGKQFKCKVNNIGLPSPIEEIISKTPGQAHQP | Protein A − |
| | NVYVLPPSRDEMSKNTVTLTCLVKDFFPPEIDVEWQSNG | C1q + |
| | QQEPESKYRMTPPQLDEDGSYFLYSKLSVDKSRWQRGDT | CD16 − |
| | FICAVMHEALHNHYTQISLSHSPGK | D(38)G |
| | | K(97)I |
| | | A(98)G |
| 82 | PGCGLLGGPSVFIFPPKPKDTLLIARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCRVNNKALPSPIEEIISKTPGQAHQP | C1q − |
| | NVYVLPPSRDEMSKNTVTLTCLVKDFFPPEIDVEWQSNG | K(93)R |
| | QQEPESKYRMTPPQLDEDGSYFLYSKLSVDKSRWQRGDT | Protein A + |
| | FICAVMHEALHNHYTQISLSHSPGK | I(21)T |
| | | V(23)L |
| | | T(24)I |
| 83 | PAPEMLGGPSVFIFPPKPKDTLLIARTPEVTCVVVDLGP | Exemplary variant |
| | EDPEVQISWFVDGKQMQTAKTQPREEQFNGTYRVVSVLP | canine IgG-B Fc |
| | IGHQDWLKGKQFTCRVNNIGLPSPIERTISKARGQAHQP | Protein A + |
| | SVYVLPPSREELSKNTVSLTCLIKDFFPPDIDVEWQSNG | C1q − |
| | QQEPESKYRTTPPQLDEDGSYFLYSKLSVDKSRWQRGDT | CD16 − |
| | FICAVMHEALHNHYTQESLSHSPGK | D(38)G |
| | | K(93)R |
| | | K(97)I |
| | | A(98)G |
| 84 | PAPEPLGGPSVFIFPPKPKDTLLIARTPEVTCVVVDLDR | Exemplary variant |
| | EDPEVQISWFVDGKQMQTAKTQPREEQFNGTYRVVSVLP | canine IgG-B Fc |
| | IGHQDWLKGKQFTCRVNNKALPSPIERTISKARGQAHQP | Protein A + |
| | SVYVLPPSREELSKNTVSLTCLIKDFFPPDIDVEWQSNG | C1q − |
| | QQEPESKYRTTPPQLDEDGSYFLYSKLSVDKSRWQRGDT | CD16 − |
| | FICAVMHEALHNHYTQESLSHSPGK | M(5)P |
| | | P(39)R |
| | | K(93)R |
| 85 | PGCGLLGGPSVFIFPPKPKDILVTARTPTVTCVVVDLGP | Exemplary variant |
| | ENPEVQISWFVDSKQVQTANTQPREEQSNGTYRVVSVLP | canine IgG-C Fc |
| | IGHQDWLSGKQFKCRVNNIGLPSPIEEIISKTPGQAHQP | Protein A − |
| | NVYVLPPSRDEMSKNTVTLTCLVKDFFPPEIDVEWQSNG | C1q − |
| | QQEPESKYRMTPPQLDEDGSYFLYSKLSVDKSRWQRGDT | CD16 − |
| | FICAVMHEALHNHYTQISLSHSPGK | D(38)G |
| | | K(93)R |
| | | K(97)I |
| | | A(98)G |
| 86 | PGCGPLGGPSVFIFPPKPKDILVTARTPTVTCVVVDLDR | Exemplary variant |
| | ENPEVQISWFVDSKQVQTANTQPREEQSNGTYRVVSVLP | canine IgG-C Fc |
| | IGHQDWLSGKQFKCRVNNKALPSPIEEIISKTPGQAHQP | Protein A − |
| | NVYVLPPSRDEMSKNTVTLTCLVKDFFPPEIDVEWQSNG | C1q − |
| | QQEPESKYRMTPPQLDEDGSYFLYSKLSVDKSRWQRGDT | CD16 − |
| | FICAVMHEALHNHYTQISLSHSPGK | M(5)P |
| | | P(39)R |
| | | K(93)R |
| 87 | GGPSVFLFPPNPKDTLMITRTPEVTCVVVDVSQENPDVK | Exemplary wild-type |
| | FNWYMDGVEVRTATTRPKEEQFNSTYRVVSVLRIQHQDW | equine IgG1 Fc |
| | LSGKEFKCKVNNQALPQPIERTITKTKGRSQEPQVYVLA | Protein A + |
| | PHPDESKKSKVSVTCLVKDFYPPEINIEWQSNGQPELET | C1q + |
| | KYSTTQAQQDSDGSYFLYSKLSVDRNRWQQGTTFTCGVM | |
| | HEALHNHYTQKNVSKNPGK | |
| 88 | GGPSVFIFPPNPKDALMISRTPVVTCVVVNLSDQYPDVQ | Exemplary wild-type |
| | FSWYVDNTEVHSAITKQREAQFNSTYRVVSVLPIQHQDW | equine IgG2 Fc |
| | LSGKEFKCSVTNVGVPQPISRAISRGKGPSRVPQVYVLP | Protein A − |
| | PHPDELAKSKVSVTCLVKDFYPPDISVEWQSNRWPELEG | C1q − |
| | KYSTTPAQLDGDGSYFLYSKLSLETSRWQQVESFTCAVM | |
| | HEALHNHFTKTDISESLGK | |
| 89 | PPCVLSAEGVIPIPSVPKPQCPPYTHSKFLGGPSVFIFP | Exemplary wild-type |
| | PNPKDALMISRTPVVTCVVVNLSDQYPDVQFSWYVDNTE | equine IgG2 Fc with |
| | VHSAITKQREAQFNSTYRVVSVLPIQHQDWLSGKEFKCS | hinge |
| | VTNVGVPQPISRAISRGKGPSRVPQVYVLPPHPDELAKS | Protein A − |
| | KVSVTCLVKDFYPPDISVEWQSNRWPELEGKYSTTPAQL | C1q − |
| | DGDGSYFLYSKLSLETSRWQQVESFTCAVMHEALHNHFT | |
| | KTDISESLGK | |
| 90 | GGPSVFIFPPKPKDVLMITRMPEVTCLVVDVSHDSSDVL | Exemplary wild-type |
| | FTWYVDGTEVKTAKTMPNEEQNNSTYRVVSVLRIQHQDW | equine IgG3 Fc |
| | LNGKKFKCKVNNQALPAPVERTISKATGQTRVPQVYVLA | Protein A + |
| | PHPDELSKNKVSVTCLVKDFYPPDITVEWQSNEHPEPEG | C1q + |
| | KYRTTEAQKDSDGSYFLYSKLTVEKDRWQQGTTFTCVVM | |
| | HEALHNHVMQKNISKNPGK | |
| 91 | VGPSVFIFPPKPKDVLMISRTPTVTCVVVDVGHDFPDVQ | Exemplary wild-type |
| | FNWYVDGVETHTATTEPKQEQFNSTYRVVSVLPIQHKDW | equine IgG4 Fc |
| | LSGKEFKCKVNNKALPAPVERTISAPTGQPREPQVYVLA | Protein A + |
| | PHRDELSKNKVSVTCLVKDFYPPDIDIEWKSNGQPEPET | C1q + |
| | KYSTTPAQLDSDGSYFLYSKLTVETNRWQQGTTFTCAVM | |
| | HEALHNHYTEKSVSKSPGK | |
| 92 | GGPSVFIFPPKPKDVLMISRKPEVTCVVVDLGHDDPDVQ | Exemplary wild-type |
| | FTWFVDGVETHTATTEPKEEQFNSTYRVVSVLPIQHQDW | equine IgG5 Fc |
| | LSGKEFKCSVTSKALPAPVERTISKAKGQLRVPQVYVLA | Protein A − |
| | PHPDELAKNTVSVTCLVKDFYPPEIDVEWQSNEHPEPEG | C1q − |
| | KYSTTPAQLNSDGSYFLYSKLSVETSRWKQGESFTCGVM | |
| | HEAVENHYTQKNVSHSPGK | |
| 93 | GRPSVFIFPPNPKDTLMISRTPEVTCVVVDVSQENPDVK | Exemplary wild-type |
| | FNWYVDGVEAHTATTKAKEKQDNSTYRVVSVLPIQHQDW | equine IgG6 Fc |
| | RRGKEFKCKVNNRALPAPVERTITKAKGELQDPQVYILA | Protein A − |
| | PHPDEVTKNTVSVTCLVKDFYPPDINVEWQSNEEPEPEV | C1q − |
| | KYSTTPAQLDGDGSYFLYSKLTVETDRWEQGESFTCVVM | |
| | HEAIRHTYRQKSITNFPGK | |
| 94 | VGPSVFIFPPKPKDVLMISRTPTVTCVVVDVGHDFPDVQ | Exemplary wild-type |
| | FNWYVDGVETHTATTEPKQEQNNSTYRVVSILAIQHKDW | equine IgG7 Fc |
| | LSGKEFKCKVNNQALPAPVQKTISKPTGQPREPQVYVLA | Protein A + |
| | PHPDELSKNKVSVTCLVKDFYPPDIDIEWKSNGQPEPET | C1q + |
| | KYSTTPAQLDGDGSYFLYSKLTVETNRWQQGTTFTCAVM | |
| | HEALHNHYTEKSVSKSPGK | |
| 95 | GGPSVFIFPPNPKDALMISRTPVVTCVVVNLSDQYPDVQ | Exemplary variant |
| | FSWYVDNTEVHSAITKQREAQFNSTYRVVSVLPIQHQDW | equine IgG2 Fc |
| | LSGKEFKCSVTNVGVPQPISRAISRGKGPSRVPQVYVLP | C1q − |
| | PHPDELAKSKVSVTCLVKDFYPPDISVEWQSNRWPELEG | Protein A + |
| | KYSTTPAQLDGDGSYFLYSKLSLETSRWQQGESFTCAVM | F(203)Y |
| | HEALHNHYTKTDISESLGK | |
| 96 | GGPSVFIFPPNPKDTLMISRTPVVTCVVVNLSDQYPDVQ | Exemplary variant |
| | FSWYVDNTEVHSAITKQREAQFNSTYRVVSVLPIQHQDW | equine IgG2 Fc |
| | LSGKEFKCSVTNVGVPQPISRAISRGKGPSRVPQVYVLP | C1q − |
| | PHPDELAKSKVSVTCLVKDFYPPDISVEWQSNRWPELEG | Protein A + |
| | KYSTTPAQLDGDGSYFLYSKLSLETSRWQQVESFTCAVM | A(15)T |
| | HEALHNHYTKTDISESLGK | F(203)Y |
| 97 | GGPSVFIFPPKPKDVLMISRKPEVTCVVVDLGHDDPDVQ | Exemplary variant |
| | FTWFVDGVETHTATTEPKEEQFNSTYRVVSVLPIQHQDW | equine IgG5 Fc |
| | LSGKEFKCSVTSKALPAPVERTISKAKGQLRVPQVYVLA | C1q − |
| | PHPDELAKNIVSVICLVKDFYPPEIDVEWQSNEHPEPEG | Protein A + |
| | KYSTTPAQLNSDGSYFLYSKLSVETSRWKQGESFTCGVM | V(199)L |
| | HEALHNHYTQKNVSHSPGK | E(200)H |
| 98 | GRPSVFIFPPNPKDTLMISRTPEVTCVVVDVSQENPDVK | Exemplary variant |
| | FNWYVDGVEAHTATTKAKEKQDNSTYRVVSVLPIQHQDW | equine IgG6 Fc |
| | RRGKEFKCKVNNRALPAPVERTITKAKGELQDPQVYILA | C1q − |
| | PHPDEVTKNTVSVTCLVKDFYPPDINVEWQSNEEPEPEV | Protein A + |
| | KYSTTPAQLDGDGSYFLYSKLTVETDRWEQGESFTCVVM | I(199)L |
| | HEALHNHYRQKSITNFPGK | R(200)H |
| | | H(201)N |
| | | T(202)H |
| 99 | GGPSVFLFPPNPKDTLMITRTPEVTCVVVDVSQENPDVK | Exemplary variant |
| | FNWYMDGVEVRTATTRPKEEQFNSTYRVVSVLRIQHQDW | equine IgG1 Fc |
| | LSGKEFKCSVNNQALPQPIERTITKTKGRSQEPQVYVLA | Protein A + |
| | PHPDESKKSKVSVTCLVKDFYPPEINIEWQSNGQPELET | C1q − |
| | KYSTTQAQQDSDGSYFLYSKLSVDRNRWQQGTTFTCGVM | K(87)S |
| | HEALHNHYTQKNVSKNPGK | |
| 100 | GGPSVFIFPPKPKDVLMITRMPEVTCLVVDVSHDSSDVL | Exemplary variant |
| | FTWYVDGTEVKTAKTMPNEEQNNSTYRVVSVLRIQHQDW | equine IgG3 Fc |
| | LNGKKFKCSVNNQALPAPVERTISKATGQTRVPQVYVLA | Protein A + |
| | PHPDELSKNKVSVTCLVKDFYPPDITVEWQSNEHPEPEG | C1q − |
| | KYRTTEAQKDSDGSYFLYSKLTVEKDRWQQGTTFTCVVM | K(87)S |
| | HEALHNHVMQKNISKNPGK | |
| 101 | VGPSVFIFPPKPKDVLMISRTPTVTCVVVDVGHDFPDVQ | Exemplary variant |
| | FNWYVDGVETHTATTEPKQEQFNSTYRVVSVLPIQHKDW | equine IgG4 Fc |
| | LSGKEFKCSVNNKALPAPVERTISAPTGQPREPQVYVLA | Protein A + |
| | PHRDELSKNKVSVTCLVKDFYPPDIDIEWKSNGQPEPET | C1q − |
| | KYSTTPAQLDSDGSYFLYSKLIVETNRWQQGTTFTCAVM | K(87)S |
| | HEALHNHYTEKSVSKSPGK | |
| 102 | VGPSVFIFPPKPKDVLMISRTPTVTCVVVDVGHDFPDVQ | Exemplary variant |
| | FNWYVDGVETHTATTEPKQEQNNSTYRVVSILAIQHKDW | equine IgG7 Fc |
| | LSGKEFKCSVNNQALPAPVQKTISKPTGQPREPQVYVLA | Protein A + |
| | PHPDELSKNKVSVTCLVKDFYPPDIDIEWKSNGQPEPET | C1q − |
| | KYSTTPAQLDGDGSYFLYSKLTVETNRWQQGTTFTCAVM | K(87)S |
| | HEALHNHYTEKSVSKSPGK | |
| 103 | RKTDHPPGPKTGEGPKCPPPEMLGGPSIFIFPPKPKDTL | Exemplary wild-type |
| | SISRTPEVTCLVVDLGPDDSDVQITWFVDNTQVYTAKTS | feline IgG1a Fc |
| | PREEQFNSTYRVVSVLPILHQDWLKGKEFKCKVNSKSLP | Protein A + |
| | SPIERTISKAKGQPHEPQVYVLPPAQEELSENKVSVTCL | C1q + |
| | IKSFHPPDIAVEWEITGQPEPENNYRTTPPQLDSDGTYF | |
| | VYSKLSVDRSHWQRGNTYTCSVSHEALHSHHTQKSLTQS | |
| | PGK | |
| 104 | RKTDHPPGPKPCDCPKCPPPEMLGGPSIFIFPPKPKDTL | Exemplary wild-type |
| | SISRTPEVTCLVVDLGPDDSDVQITWFVDNTQVYTAKTS | feline IgG1a Fc |
| | PREEQFNSTYRVVSVLPILHQDWLKGKEFKCKVNSKSLP | Protein A + |
| | SPIERTISKAKGQPHEPQVYVLPPAQEELSENKVSVTCL | C1q + |
| | IKSFHPPDIAVEWEITGQPEPENNYRTTPPQLDSDGTYF | |
| | VYSKLSVDRSHWQRGNTYTCSVSHEALHSHHTQKSLTQS | |
| | PGK | |
| 105 | RKTDHPPGPKTGEGPKCPPPEMLGGPSIFIFPPKPKDTL | Exemplary wild-type |
| | SISRTPEVTCLVVDLGPDDSDVQITWFVDNTQVYTAKTS | feline IgG1b Fc |
| | PREEQFNSTYRVVSVLPILHQDWLKGKEFKCKVNSKSLP | Protein A+ |
| | SPIERTISKDKGQPHEPQVYVLPPAQEELSENKVSVTCL | C1q + |
| | IEGFYPSDIAVEWEITGQPEPENNYRTTPPQLDSDGTYF | |
| | LYSRLSVDRSRWQRGNTYTCSVSHEALHSHHTQKSLTQS | |
| | PGK | |
| 106 | RKTDHPPGPKPCDCPKCPPPEMLGGPSIFIFPPKPKDTL | Exemplary wild-type |
| | SISRTPEVTCLVVDLGPDDSDVQITWFVDNTQVYTAKTS | feline IgG1b Fc |
| | PREEQFNSTYRVVSVLPILHQDWLKGKEFKCKVNSKSLP | Protein A + |
| | SPIERTISKDKGQPHEPQVYVLPPAQEELSENKVSVTCL | C1q + |
| | IEGFYPSDIAVEWEITGQPEPENNYRTTPPQLDSDGTYF | |
| | LYSRLSVDRSRWQRGNTYTCSVSHEALHSHHTQKSLTQS | |
| | PGK | |
| 107 | PKTASTIESKTGEGPKCPVPEIPGAPSVFIFPPKPKDTL | Exemplary wild-type |
| | SISRTPEVTCLVVDLGPDDSNVQITWFVDNTEMHTAKTR | feline IgG2 Fc |
| | PREEQFNSTYRVVSVLPILHQDWLKGKEFKCKVNSKSLP | Protein A + |
| | SAMERTISKAKGQPHEPQVYVLPPTQEELSENKVSVTCL | C1q − |
| | IKGFHPPDIAVEWEITGQPEPENNYQTTPPQLDSDGTYF | |
| | LYSRLSVDRSHWQRGNTYTCSVSHEALHSHHTQKSLTQS | |
| | PGK | |
| 108 | RKTDHPPGPKPCDCPKCPPPEMLGGPSIFIFPPKPKDTL | Exemplary variant |
| | SISRTPEVTCLVVDLGPDDSDVQITWFVDNTQVYTAKTS | feline IgG1a Fc |
| | PREEQFNSTYRVVSVLPILHQDWLKGKEFKCKVNSKSLP | Protein A + |
| | SPIERTISKAKGQPHEPQVYVLPPAQEELSENKVSVICL | C1q − |
| | IKSFHPPDIAVEWEITGQPEPENNYRTTPPQLDSDGTYF | P(198)A |
| | VYSKLSVDRSHWQRGNTYTCSVSHEALHSHHTQKSLTQS | |
| | PGK | |
| 109 | RKTDHPPGPKTGEGPKCPPPEMLGGPSIFIFPPKPKDTL | Exemplary variant |
| | SISRTPEVTCLVVDLGPDDSDVQITWFVDNTQVYTAKTS | feline IgG1a Fc |
| | PREEQFNSTYRVVSVLPILHQDWLKGKEFKCKVNSKSLP | Protein A + |
| | SPIERTISKAKGQPHEPQVYVLPPAQEELSENKVSVTCL | C1q − |
| | IKSFHPPDIAVEWEITGQPEPENNYRTTPPQLDSDGTYF | P(198)A |
| | VYSKLSVDRSHWQRGNTYTCSVSHEALHSHHTQKSLTQS | |
| | PGK | |
| 110 | RKTDHPPGPKPCDCPKCPPPEMLGGPSIFIFPPKPKDTL | Exemplary variant |
| | SISRTPEVTCLVVDLGPDDSDVQITWFVDNTQVYTAKTS | feline IgG1b Fc |
| | PREEQFNSTYRVVSVLPILHQDWLKGKEFKCKVNSKSLP | Protein A + |
| | SPIERTISKDKGQPHEPQVYVLPPAQEELSENKVSVTCL | C1q − |
| | IEGFYPSDIAVEWEITGQPEPENNYRTTPPQLDSDGTYF | P(198)A |
| | LYSRLSVDRSRWQRGNTYTCSVSHEALHSHHTQKSLTQS | |
| | PGK | |
| 111 | RKTDHPPGPKTGEGPKCPPPEMLGGPSIFIFPPKPKDTL | Exemplary variant |
| | SISRTPEVTCLVVDLGPDDSDVQITWFVDNTQVYTAKTS | feline IgG1b Fc |
| | PREEQFNSTYRVVSVLPILHQDWLKGKEFKCKVNSKSLP | Protein A + |
| | SPIERTISKDKGQPHEPQVYVLPPAQEELSENKVSVTCL | C1q − |
| | IEGFYPSDIAVEWEITGQPEPENNYRTTPPQLDSDGTYF | P(198)A |
| | LYSRLSVDRSRWQRGNTYTCSVSHEALHSHHTQKSLTQS | |
| | PGK | |
| 112 | [symbol] APPRLICDSRVLE | Exemplary feline |
| | RYILEAREAENVTMGCNETCSFSENITVPDTKVNFYTWK | EPO Analog 6-30 |
| | RMDVGQQAVEVWQGLALLSEAILRGQALLANSSQVNETL | EV Precursor |
| | QLHVDKAVSSLRSLTSLLRALGAQKEATSLPEATSAAPL | |
| | RTFTVDTLCKLFRIYSNFLRGKLTLYTGEACRRGDR | |
| 113 | APPRLICDSRVLERYILEAREAENVTMGCNETCSFSENI | Exemplary feline |
| | TVPDTKVNFYTWKRMDVGQQAVEVWQGLALLSEAILRGQ | EPO Analog 6-30 |
| | ALLANSSQVNETLQLHVDKAVSSLRSLTSLLRALGAQKE | EV Mature |
| | ATSLPEATSAAPLRTFTVDTLCKLFRIYSNFLRGKLTLY | |
| | TGEACRRGDR | |
| 114 | MGSCECPALLLLLSLLLLPLGLPVLGAPPRLICDSRVLE | Exemplary feline |
| | RYILEAREAENVTMGCAEGCSFSENITVPDTKVNFYTWK | EPO analog 14 |
| | RMNVTQQAVEVWQGLALLSEAILRGQALLANSSQPSETL | precursor |
| | QLHVDKAVSSLRSLTSLLRALGAQKEATSLPEATSAAPL | D81N |
| | RTFTVDTLCKLFRIYSNFLRGKLTLYTGEACRRGDR | G83T |
| 115 | APPRLICDSRVLERYILEAREAENVTMGCAEGCSFSENI | Exemplary feline |
| | TVPDTKVNFYTWKRMNVTQQAVEVWQGLALLSEAILRGQ | EPO analog 14 |
| | ALLANSSQPSETLQLHVDKAVSSLRSLTSLLRALGAQKE | mature |
| | ATSLPEATSAAPLRTFTVDTLCKLFRIYSNFLRGKLTLY | D55N |
| | TGEACRRGDR | G57T |
| 116 | MGSCECPALLLLLSLLLLPLGLPVLGAPPRLICDSRVLE | Exemplary feline |
| | RYILEAREAENVTMGCAEGCSFSENITVPDTKVNFYTWK | EPO analog 40 |
| | RMDVGQQAVEVWQGLALLSEAILRGQALLANSSQPSETL | precursor |
| | QLHVDKAVSSLRSLTSLLRANGTQKEATSLPEATSAAPL | L138N |
| | RTFTVDTLCKLFRIYSNFLRGKLTLYTGEACRRGDR | A140T |
| 117 | APPRLICDSRVLERYILEAREAENVTMGCAEGCSFSENI | Exemplary feline |
| | TVPDTKVNFYTWKRMDVGQQAVEVWQGLALLSEAILRGQ | EPO analog 40 |
| | ALLANSSQPSETLQLHVDKAVSSLRSLTSLLRANGTQKE | mature |
| | ATSLPEATSAAPLRTFTVDTLCKLFRIYSNFLRGKLTLY | L112N |
| | TGEACRRGDR | A114T |
| 118 | MGSCECPALLLLLSLLLLPLGLPVLGAPPRLICDSRVLE | Exemplary feline |
| | RYILEAREAENVTMGCAEGCSFSENITVPDTKVNFYTWK | EPO analog 56 |
| | RMDVGQQAVEVWQGLALLSEAILRGQALLANSSQPSETL | precursor |
| | QLHVDKAVSSLRSLTSLLRALGAQKEATSNVTATSAAPL | L147N |
| | RTFTVDTLCKLFRIYSNFLRGKLTLYTGEACRRGDR | P148V |
| | | E149T |
| 119 | APPRLICDSRVLERYILEAREAENVTMGCAEGCSFSENI | Exemplary feline |
| | TVPDTKVNFYTWKRMDVGQQAVEVWQGLALLSEAILRGQ | EPO analog 56 |
| | ALLANSSQPSETLQLHVDKAVSSLRSLTSLLRALGAQKE | mature |
| | ATSNVTATSAAPLRTFTVDTLCKLFRIYSNFLRGKLTLY | L121N |
| | TGEACRRGDR | P122V |
| | | E123T |
| 120 | MGSCECPALLLLLSLLLLPLGLPVLGAPPRLICDSRVLE | Exemplary feline |
| | RYILEAREAENVTMGCAEGCSFSENITVPDTKVNFYTWK | EPO analog 71 |
| | RMDVGQQAVEVWQGLALLSEAILRGQALLANSSQPSETL | precursor |
| | QLHVDKAVSSLRSLTSLLRALGAQKEATSLENATSAAPL | P148E |
| | RTFTVDTLCKLFRIYSNFLRGKLTLYTGEACRRGDR | E149N |
| 121 | APPRLICDSRVLERYILEAREAENVTMGCAEGCSFSENI | Exemplary feline |
| | TVPDTKVNFYTWKRMDVGQQAVEVWQGLALLSEAILRGQ | EPO analog 71 |
| | ALLANSSQPSETLQLHVDKAVSSLRSLTSLLRALGAQKE | mature |
| | ATSLENATSAAPLRTFTVDTLCKLFRIYSNFLRGKLTLY | P122E |
| | TGEACRRGDR | E123N |
| 122 | MGSCECPALLLLLSLLLLPLGLPVLGAPPRLICDSRVLE | Exemplary feline |
| | RYILEAREAENVTMGCAEGCSFSENITVPDTKVNFYTWK | EPO analog |
| | RMDVGQQAVEVWQGLALLSEAILRGQALLANSSQPSETL | precursor |
| | QLHVDKAVSSLRSLTSLLRALGAQKEATSLPEATSAAPL | C165X, wherein X |
| | RTFTVDTLXKLFRIYSNFLRGKLTLYTGEACRRGDR | may be any amino |
| | | acid other than C, |
| | | such as S, T, or A |
| 123 | APPRLICDSRVLERYILEAREAENVTMGCAEGCSFSENI | Exemplary feline |
| | TVPDTKVNFYTWKRMDVGQQAVEVWQGLALLSEAILRGQ | EPO analog mature |
| | ALLANSSQPSETLQLHVDKAVSSLRSLTSLLRALGAQKE | C139X, wherein X |
| | ATSLPEATSAAPLRTFTVDTLXKLFRIYSNFLRGKLTLY | may be any amino |
| | TGEACRRGDR | acid other than C, |
| | | such as S, T, or A |

### DETAILED DESCRIPTION OF THE INVENTIONS

The present disclosure provides analogs of wild-type canine EPO polypeptides (SEQ ID NO: 1: precursor form; SEQ ID NO: 2: mature form), wild-type equine EPO polypeptides (SEQ ID NO: 3: precursor form; SEQ ID NO: 4: mature form), and wild-type feline EPO E44 precursor (SEQ ID NO: 7, where E44 corresponds to E18 in the mature EPO) and wild-type feline EPO E18 mature (SEQ ID NO: 8) polypeptides having one or more additional glycosylation sites and/or one or more additional cysteine residues.

For example, amino acid locations of EPO polypeptides suitable for introducing additional N-linked glycosylation sites (singly or in any combination) are provided. Methods of producing or purifying the EPO polypeptides, including acidic and basic fractions of EPO polypeptides, are also provided as are methods of treatment using EPO polypeptides. Formulations for single dose and/or multi dose pharmaceutical compositions of EPO polypeptides, are also described. Nucleic acids, vectors, expression systems encoding EPO polypeptides and methods of expressing those polypeptides, including controlled expression, by gene therapy methods are described.

Also described herein are polypeptides comprising an extracellular domain of EPO receptor and methods of administering those EPOR polypeptides or nucleic acids encoding those EPOR polypeptides for the treatment of polycythemia in companion animals.

For the convenience of the reader, the following definitions of terms used herein are provided.

As used herein, numerical terms such as K_{d} are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### Exemplary EPO Polypeptides

Novel EPO polypeptides are provided, for example, EPO polypeptides comprising the amino acid sequence of SEQ ID NO: 1, SEQ ID NO: 2, SEQ ID NO: 3, or SEQ ID NO: 4 except for the presence of at least one N-linked glycosylation site not present in SEQ ID NO: 1, SEQ ID NO: 2, SEQ ID NO: 3, or SEQ ID NO: 13. Other examples include EPO polypeptides comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 except for the presence of at least one cysteine not present in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

“Amino acid sequence” means a sequence of amino acids in a protein, and includes sequences of amino acids in which one or more amino acids of the sequence have had their side-groups chemically modified, as well as those in which, relative to a known sequence, one or more amino acids have been replaced, inserted or deleted, without thereby eliminating a desired property, such as ability to bind EPO receptor. An amino acid sequence may also be referred to as a peptide, oligopeptide, or protein.

“Erythropoietin,” “EPO,” or “EPO polypeptide,” as used herein, is a polypeptide comprising the entirety or a fragment of EPO.

For example, “EPO” refers to an EPO polypeptide from any vertebrate source, including mammals such as primates (e.g., humans and cynomolgus monkeys), rodents (e.g., mice and rats), and companion animals (e.g., dogs, cats, and equine), unless otherwise indicated.

In some embodiments, EPO polypeptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, or SEQ ID NO: 123.

“Erythropoietin receptor,” “EPO receptor,” or “EPOR,” as used herein, is a polypeptide comprising the entirety or a portion of EPO receptor that binds to an EPO polypeptide.

For example, “EPOR” refers to an EPOR polypeptide from any vertebrate source, including mammals such as primates (e.g., humans and cynomolgus monkeys), rodents (e.g., mice and rats), and companion animals (e.g., dogs, cats, and equine), unless otherwise indicated.

In some embodiments, EPOR comprises the amino acid sequence of SEQ ID NO: 33, SEQ ID NO: 34, SEQ ID NO: 35, SEQ ID NO: 37, SEQ ID NO: 38, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, or SEQ ID NO 52.

The term “companion animal species” or “companion animal” refers to an animal suitable to be a companion to humans. In some embodiments, a companion animal is a dog, cat, or horse. In some embodiments, a companion animal is a rabbit, ferret, guinea pig, or rodent, etc. In some embodiments, a companion animal is a cow or pig.

An “extracellular domain” (“ECD”) is the portion of a polypeptide that extends beyond the transmembrane domain into the extracellular space. The term “extracellular domain,” as used herein, may comprise a complete extracellular domain or may comprise a truncated extracellular domain missing one or more amino acids, that binds to its ligand. The composition of the extracellular domain may depend on the algorithm used to determine which amino acids are in the membrane. Different algorithms may predict, and different systems may express, different extracellular domains for a given protein.

An extracellular domain of an EPOR polypeptide may comprise a complete extracellular domain or a truncated extracellular domain of EPOR that binds EPO. In some embodiments, an extracellular domain of an EPOR polypeptide is an extracellular domain of an EPOR polypeptide derived from a companion animal species. For example, in some embodiments, an extracellular domain of an EPOR polypeptide is derived from canine EPOR, feline EPOR, equine EPOR, or human EPOR.

In some embodiments, an extracellular domain of an EPOR polypeptide comprises the amino acid sequence of SEQ ID NO: 33, SEQ ID NO: 34, SEQ ID NO: 35, SEQ ID NO: 37, SEQ ID NO: 38, SEQ ID NO: 39, SEQ ID NO: 42, SEQ ID NO: 43, SEQ ID NO: 45, SEQ ID NO: 46, SEQ ID NO: 48, SEQ ID NO: 49, SEQ ID NO: 51, or SEQ ID NO: 52.

“Wild-type” refers to a non-mutated version of a polypeptide that occurs in nature, or a fragment thereof. A wild-type polypeptide may be produced recombinantly.

A “biologically active” entity, or an entity having “biological activity,” is an entity having any function related to or associated with a metabolic or physiological process, and/or having structural, regulatory, or biochemical functions of a naturally-occurring molecule. A biologically active polypeptide or fragment thereof includes one that can participate in a biological reaction, including, but not limited to, a ligand-receptor interaction or antigen-antibody binding. The biological activity can include an improved desired activity, or a decreased undesirable activity. An entity may demonstrate biological activity when it participates in a molecular interaction with another molecule, when it has therapeutic value in alleviating a disease condition, when it has prophylactic value in inducing an immune response, when it has diagnostic and/or prognostic value in determining the presence of a molecule.

An “analog” or a “variant” are used unterchangably to refer to a polypeptide that differs from a reference polypeptide by single or multiple amino acid substitutions, deletions, and/or additions that substantially retains at least one biological activity of the reference polypeptide.

As used herein, “percent (%) amino acid sequence identity” and “homology” with respect to a polypeptide sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

In some embodiments, an analog or a variant has at least about 50% amino acid sequence identity, at least about 60% amino acid sequence identity, at least about 65% amino acid sequence identity, at least about 70% amino acid sequence identity, at least about 75% amino acid sequence identity, at least about 80% amino acid sequence identity, at least about 85% amino acid sequence identity, at least about 90% amino acid sequence identity, at least about 95% amino acid sequence identity, at least about 97% amino acid sequence identity, at least about 98% amino acid sequence identity, or at least about 99% amino acid sequence identity with the wild-type or reference sequence polypeptide.

As used herein, “position corresponding to position n,” wherein n is any number, refers to an amino acid position of a subject polypeptide that aligns with position n of a reference polypeptide after aligning the amino acid sequences of the subject and reference polypeptides and introducing gaps. Alignment for purposes of whether a position of a subject polypeptide corresponds with position n of a reference polypeptide can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for alignment, including any parameters needed to achieve maximal alignment over the full length of two sequences being compared. In some embodiments, the subject polypeptide and the reference polypeptide are of different lengths.

A “point mutation” is a mutation that involves a single amino acid residue. The mutation may be the loss of an amino acid, substitution of one amino acid residue for another, or the insertion of an additional amino acid residue.

An “amino acid substitution” refers to the replacement of one amino acid in a polypeptide with another amino acid. In some embodiments, an amino acid substitution is a conservative substitution. Nonlimiting exemplary substitutions are shown in Table 2. Amino acid substitutions may be introduced into a molecule of interest and the products screened for a desired activity, for example, retained/improved receptor binding, decreased immunogenicity, or improved pharmacokinetics.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; Ala; |
| | Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

In some embodiments, the EPO polypeptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 except for the presence of at least one N-linked glycosylation site not present in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some embodiments, the at least one N-linked glycosylation site comprises the sequence asparagine-xaa-serine, wherein xaa is any amino acid except proline. In some embodiments, the at least one N-linked glycosylation site comprises the sequence asparagine-xaa-threonine, wherein xaa is any amino acid except proline. In some embodiments, the at least one N-linked glycosylation site does not overlap with another N-linked glycosylation site.

In some embodiments, the EPO polypeptide comprises an N-linked glycosylation site at amino acid positions 47-49, 55-57, 56-58, 60-62, 61-63, 79-81, 81-83, 82-84, 91-93, 92-94, 97-99, 98-100, 99-101, 112-114, 113-115, 114-116, 115-117, 116-118, 137-139, 138-140, 140-142, 141-143, 142-144, 143-145, 144-146, 145-147, 146-148, 147-149, 148-150, 149-151, 150-152, 161-163, 162-164, 184-186, and/or 186-188 of SEQ ID NO: 1, SEQ ID NO: 3, SEQ ID NO: 5, or SEQ ID NO: 7.

In some embodiments, the EPO polypeptide comprises an N-linked glycosylation site at amino acid positions 21-23, 29-31, 30-32, 34-36, 35-37, 53-55, 55-57, 56-58, 65-67, 66-68, 71-73, 72-74, 73-75, 86-88, 87-89, 88-90, 89-91, 90-92, 111-113, 112-114, 114-116, 115-117, 116-118, 117-119, 118-120, 119-121, 120-122, 121-123, 122-124, 123-125, 124-126, 135-137, 136-138, 158-160, and/or 162-164 of SEQ ID NO: 2, SEQ ID NO: 4, SEQ ID NO: 6, or SEQ ID NO: 8.

In some embodiments, the EPO polypeptide comprises an amino acid other than proline at an amino acid position corresponding to position 113 or position 148 of SEQ ID NO: 1, SEQ ID NO: 3, SEQ ID NO: 5, or SEQ ID NO: 7. In some embodiments, the EPO polypeptide comprises an amino acid other than proline at an amino acid position corresponding to position 87 or position 122 of SEQ ID NO: 2, SEQ ID NO: 4, SEQ ID NO: 6, or SEQ ID NO: 8.

In some embodiments, the EPO polypeptide comprises a valine or a glutamic acid at an amino acid position corresponding to position 113 or position 148 of SEQ ID NO: 1, SEQ ID NO: 3, SEQ ID NO: 5, or SEQ ID NO: 7. In some embodiments, the EPO polypeptide comprises a valine or a glutamic acid at an amino acid position corresponding to position 87 or position 122 of SEQ ID NO: 2, SEQ ID NO: 4, SEQ ID NO: 6, or SEQ ID NO: 8.

In some embodiments, the EPO polypeptide comprises the amino acid sequence of SEQ ID NO: 9, SEQ ID NO: 10, SEQ ID NO: 11, SEQ ID NO: 12, SEQ ID NO: 13, SEQ ID NO: 44, SEQ ID NO: 15, SEQ ID NO: 16, SEQ ID NO: 17, SEQ ID NO: 18, SEQ ID NO: 19, or SEQ ID NO: 20.

In some embodiments, the EPO polypeptide comprises one or more amino acid modifications listed in Table 3, Table 4, or Table 5, below.

TABLE 3

| | Amino acid substitutions for N-linked glycosylation sites | |
|---|---|---|
| | Based on wt canine EPO | Based on wt canine EPO |
| Analog No. | sequence (SEQ ID NO: 1) | sequence (SEQ ID NO: 2) |
| 1 | N47S49 | N21S23 |
| 2 | N47T49 | N21T23 |
| 3 | N55S57 | N29S31 |
| 4 | N55T57 | N29T31 |
| 5 | N56S58 | N30S32 |
| 6 | N56T58 | N30T32 |
| 7 | N60 | N34 |
| 8 | N60T62 | N34T36 |
| 9 | N61S63 | N35S37 |
| 10 | N61T63 | N35T37 |
| 11 | N79S81 | N53S55 |
| 12 | N79T81 | N53T55 |
| 13 | N81S83 | N55S57 |
| 14 | N81T83 | N55T57 |
| 15 | N82S84 | N56S58 |
| 16 | N82T84 | N56T58 |
| 17 | N91S93 | N65S67 |
| 18 | N91T93 | N65T67 |
| 19 | N92S94 | N66S68 |
| 20 | N92T94 | N66T68 |
| 21 | N97S99 | N71S73 |
| 22 | N97T99 | N71T73 |
| 23 | N98S100 | N72S74 |
| 24 | N98T100 | N72T74 |
| 25 | N99S101 | N73S75 |
| 26 | N99T101 | N73T75 |
| 27 | N112*X113 | N86*X87 |
| 28 | N112*X113T114 | N86*X87T88 |
| 29 | N113S115 | N87S89 |
| 30 | N113T115 | N87T89 |
| 31 | *X113N114S116 | *X87N88S90 |
| 32 | *X113N114 | *X87N88 |
| 33 | N115S117 | N89S91 |
| 34 | N115T117 | N89T91 |
| 35 | N116*X117S118 | N90*X91S92 |
| 36 | N116*X117T118 | N90*X91T92 |
| 37 | N137S139 | N111S113 |
| 38 | N137T139 | N111T113 |
| 39 | N138S140 | N112S114 |
| 40 | N138T140 | N112T114 |
| 41 | N140S142 | N114S116 |
| 42 | N140T142 | N114T116 |
| 43 | N141S143 | N115S117 |
| 44 | N141T143 | N115T117 |
| 45 | N142S144 | N116S118 |
| 46 | N142T144 | N116T118 |
| 47 | N143S145 | N117S119 |
| 48 | N143T145 | N117T119 |
| 49 | N144 | N118 |
| 50 | N144T146 | N118T120 |
| 51 | N145S147 | N119S121 |
| 52 | N145T147 | N119T121 |
| 53 | N146S148 | N120S122 |
| 54 | N146T148 | N120T122 |
| 55 | N147*X148S149 | N121*X122S123 |
| 56 | N147*X148T149 | N121*X122T123 |
| 57 | N148S150 | N122S124 |
| 58 | N148T150 | N122T124 |
| 59 | N149S151 | N123S125 |
| 60 | N149T151 | N123T125 |
| 71 | *X148N149S151 | *X122N123S125 |
| 72 | *X148N149T151 | *X122N123T125 |
| 61 | N150 | N124 |
| 62 | N150T152 | N124T126 |
| 63 | N161S163 | N135S137 |
| 64 | N161 | N135 |
| 65 | N162S164 | N136S138 |
| 66 | N162T164 | N136T138 |
| 67 | N184S186 | N158S160 |
| 68 | N184T186 | N158T160 |
| 69 | N186S188 | N162S164 |
| 70 | N186T188 | N162T164 |

*X indicates any amino acid except proline (such as E, V, S, A, etc.).

TABLE 4

| | Amino acid substitutions for N-linked glycosylation sites | |
|---|---|---|
| | Based on wt equine EPO | Based on wt equine EPO |
| Analog No. | sequence (SEQ ID NO: 3) | sequence (SEQ ID NO: 4) |
| 1 | N47S49 | N21S23 |
| 2 | N47T49 | N21T23 |
| 3 | N55S57 | N29S31 |
| 4 | N55T57 | N29T31 |
| 5 | N56S58 | N30S32 |
| 6 | N56T58 | N30T32 |
| 7 | N60S62 | N34S36 |
| 8 | N60T62 | N34T36 |
| 9 | N61S63 | N35S37 |
| 10 | N61T63 | N35T37 |
| 11 | N79S81 | N53S55 |
| 12 | N79T81 | N53T55 |
| 13 | N81S83 | N55S57 |
| 14 | N81T83 | N55T57 |
| 15 | N82S84 | N56S58 |
| 16 | N82T84 | N56T58 |
| 17 | N91S93 | N65S67 |
| 18 | N91T93 | N65T67 |
| 19 | N92S94 | N66S68 |
| 20 | N92T94 | N66T68 |
| 21 | N97S99 | N71S73 |
| 22 | N97T99 | N71T73 |
| 23 | N98S100 | N72S74 |
| 24 | N98T100 | N72T74 |
| 25 | N99S101 | N73S75 |
| 26 | N99T101 | N73T75 |
| 27 | N112*X113 | N86*X87 |
| 28 | N112*X113T114 | N86*X87T88 |
| 29 | N113S115 | N87S89 |
| 30 | N113T115 | N87T89 |
| 31 | *X113N114S116 | *X87N88S90 |
| 32 | *X113N114 | *X87N88 |
| 33 | N115S117 | N89S91 |
| 34 | N115T117 | N89T91 |
| 35 | N116S118 | N90S92 |
| 36 | N116T118 | N90T92 |
| 37 | N137S139 | N111S113 |
| 38 | N137T139 | N111T113 |
| 39 | N138S140 | N112S114 |
| 40 | N138T140 | N112T114 |
| 41 | N140S142 | N114S116 |
| 42 | N140T142 | N114T116 |
| 43 | N141S143 | N115S117 |
| 44 | N141T143 | N115T117 |
| 45 | N142S144 | N116S118 |
| 46 | N142T144 | N116T118 |
| 47 | N143S145 | N117S119 |
| 48 | N143T145 | N117T119 |
| 49 | N144 | N118 |
| 50 | N144T146 | N118T120 |
| 51 | N145S147 | N119S121 |
| 52 | N145T147 | N119T121 |
| 53 | N146*X147S148 | N120*X121S122 |
| 54 | N146*X147T148 | N120*X121T122 |
| 55 | N147*X148S149 | N121*X122S123 |
| 56 | N147*X148T149 | N121*X122T123 |
| 57 | N148S150 | N122S124 |
| 58 | N148T150 | N122T124 |
| 59 | N149S151 | N123S125 |
| 60 | N149T151 | N123T125 |
| 71 | *X148N149S151 | *X122N123S125 |
| 72 | *X148N149T151 | *X122N123T125 |
| 61 | N150 | N124 |
| 62 | N150T152 | N124T126 |
| 63 | N161S163 | N135S137 |
| 64 | N161 | N135 |
| 65 | N162S164 | N136S138 |
| 66 | N162T164 | N136T138 |
| 67 | N184S186 | N158S160 |
| 68 | N184T186 | N158T160 |
| 69 | N186S188 | N162S164 |
| 70 | N186T188 | N162T164 |

*X indicates any amino acid except proline (such as E, V, S, A, etc.).

TABLE 5

| | Amino acid substitutions for N-linked glycosylation sites | |
|---|---|---|
| | Based on wt feline | Based on wt feline |
| | EPO E44 precursor sequence | EPO E18 mature sequence |
| Analog No. | (SEQ ID NO: 7) | (SEQ ID NO: 8) |
| 1 | N47S49 | N21S23 |
| 2 | N47T49 | N21T23 |
| 3 | N55S57 | N29S31 |
| 4 | N55T57 | N29T31 |
| 5 | N56S58 | N30S32 |
| 6 | N56T58 | N30T32 |
| 7 | N60 | N34 |
| 8 | N60T62 | N34T36 |
| 9 | N61S63 | N35S37 |
| 10 | N61T63 | N35T37 |
| 11 | N79S81 | N53S55 |
| 12 | N79T81 | N53T55 |
| 13 | N81S83 | N55S57 |
| 14 | N81T83 | N55T57 |
| 15 | N82S84 | N56S58 |
| 16 | N82T84 | N56T58 |
| 17 | N91S93 | N65S67 |
| 18 | N91T93 | N65T67 |
| 19 | N92S94 | N66S68 |
| 20 | N92T94 | N66T68 |
| 21 | N97S99 | N71S73 |
| 22 | N97T99 | N71T73 |
| 23 | N98S100 | N72S74 |
| 24 | N98T100 | N72T74 |
| 25 | N99S101 | N73S75 |
| 26 | N99T101 | N73T75 |
| 27 | N112*X113 | N86*X87 |
| 28 | N112*X113T114 | N86*X87T88 |
| 29 | N113S115 | N87S89 |
| 30 | N113T115 | N87T89 |
| 31 | *X113N114S116 | *X87N88S90 |
| 32 | *X113N114 | *X87N88 |
| 33 | N115S117 | N89S91 |
| 34 | N115T117 | N89T91 |
| 35 | N116S118 | N90S92 |
| 36 | N116T118 | N90T92 |
| 37 | N137S139 | N111S113 |
| 38 | N137T139 | N111T113 |
| 39 | N138S140 | N112S114 |
| 40 | N138T140 | N112T114 |
| 41 | N140S142 | N114S116 |
| 42 | N140T142 | N114T116 |
| 43 | N141S143 | N115S117 |
| 44 | N141T143 | N115T117 |
| 45 | N142S144 | N116S118 |
| 46 | N142T144 | N116T118 |
| 47 | N143S145 | N117S119 |
| 48 | N143 | N117 |
| 49 | N144 | N118 |
| 50 | N144T146 | N118T120 |
| 51 | N145S147 | N119S121 |
| 52 | N145T147 | N119T121 |
| 53 | N146S148 | N120S122 |
| 54 | N146T148 | N120T122 |
| 55 | N147*X148S149 | N121*X122S123 |
| 56 | N147*X148T149 | N121*X122T123 |
| 57 | N148S150 | N122S124 |
| 58 | N148T150 | N122T124 |
| 59 | N149S151 | N123S125 |
| 71 | *X148N149 | *X122N123 |
| 72 | *X148N149S151 | *X122N123S125 |
| 60 | N149 | N123 |
| 61 | N150 | N124 |
| 62 | N150T152 | N124T126 |
| 63 | N161S163 | N135S137 |
| 64 | N161 | N135 |
| 65 | N162S164 | N136S138 |
| 66 | N162T164 | N136T138 |
| 67 | N184S186 | N158S160 |
| 68 | N184T186 | N158T160 |
| 69 | N186S188 | N162S164 |
| 70 | N186T188 | N162T164 |

*X indicates any amino acid except proline (such as E, V, S, A, etc.).

In some embodiments, the EPO polypeptide comprises the amino acid sequence of SEQ ID NO: 1, SEQ ID NO: 2, SEQ ID NO: 3, SEQ ID NO: 4, SEQ ID NO: 7, or SEQ ID NO: 8 except for the presence of at least one cysteine not present in SEQ ID NO: 1, SEQ ID NO: 2, SEQ ID NO: 3, SEQ ID NO: 4, SEQ ID NO: 7, or SEQ ID NO: 8.

In some embodiments, the EPO polypeptide comprises a cysteine at position 45, 48, 49, 68, 86, 90, 92, 120, 143, 144, and/or 172 of SEQ ID NO: 1, SEQ ID NO: 3, or SEQ ID NO: 7.

In some embodiments, the EPO polypeptide comprises a cysteine at position 19, 22, 23, 42, 60, 64, 66, 94, 117, 118, and/or 146 of SEQ ID NO: 2, SEQ ID NO: 4, or SEQ ID NO: 8.

In some embodiments, the EPO polypeptide comprises a cysteine at position 45 and a cysteine at position 172 of SEQ ID NO: 1, SEQ ID NO: 3, or SEQ ID NO: 7; or a cysteine at position 19 and a cysteine at position 146 of SEQ ID NO: 2, SEQ ID NO: 4, or SEQ ID NO: 8.

In some embodiments, the EPO polypeptide comprises a cysteine at position 48 and a cysteine at position 120 of SEQ ID NO: 1, SEQ ID NO: 3, or SEQ ID NO: 7; or a cysteine at position 22 and a cysteine at position 94 of SEQ ID NO: 2, SEQ ID NO: 4, or SEQ ID NO: 8.

In some embodiments, the EPO polypeptide comprises a cysteine at position 49 and a cysteine at position 172 of SEQ ID NO: 1, SEQ ID NO: 3, or SEQ ID NO: 7; or a cysteine at position 23 and a cysteine at position 146 of SEQ ID NO: 2, SEQ ID NO: 4, or SEQ ID NO: 8.

In some embodiments, the EPO polypeptide comprises a cysteine at position 68 and a cysteine at position 92 of SEQ ID NO: 1, SEQ ID NO: 3, or SEQ ID NO: 7; or a cysteine at position 42 and a cysteine at position 66 of SEQ ID NO: 2, SEQ ID NO: 4, or SEQ ID NO: 8.

In some embodiments, the EPO polypeptide comprises a cysteine at position 90 and a cysteine at position 144 of SEQ ID NO: 1, SEQ ID NO: 3, or SEQ ID NO: 7; or a cysteine at position 64 and a cysteine at position 118 of SEQ ID NO: 2, SEQ ID NO: 4, or SEQ ID NO: 8.

In some embodiments, the EPO polypeptide comprises a cysteine at position 86 and a cysteine at position 143 of SEQ ID NO: 1, SEQ ID NO: 3, or SEQ ID NO: 7; or a cysteine at position 60 and a cysteine at position 117 of SEQ ID NO: 2, SEQ ID NO: 4, or SEQ ID NO: 8.

In some embodiments, the EPO polypeptide comprises the amino acid sequence of SEQ ID NO: 21, SEQ ID NO: 22, SEQ ID NO: 23, SEQ ID NO: 24, SEQ ID NO: 25, SEQ ID NO: 26 SEQ ID NO: 27, SEQ ID NO: 28, SEQ ID NO: 29, SEQ ID NO: 30, SEQ ID NO: 31, or SEQ ID NO: 32.

In some embodiments, the EPO polypeptide comprises an amino acid other than a cysteine at a position corresponding to position 165 of SEQ ID NO: 7 or at a position corresponding to position 139 of SEQ ID NO: 8. In some embodiments, the amino acid other than a cysteine is a threonine, a serine, or an alanine.

In some embodiments, the EPO polypeptide comprises the amino acid sequence of SEQ ID NO: 7 or SEQ ID NO: 8 except for the presence of an amino acid other than a cysteine at position 165 of SEQ ID NO: 7 or at position 139 of SEQ ID NO: 8. In some embodiments, the amino acid other than a cysteine is a threonine, a serine, or an alanine.

In some embodiments, the EPO polypeptide comprises the amino acid sequence of SEQ ID NO: 122 or SEQ ID NO: 123, wherein X is an amino acid other than a cysteine, such as a threonine, a serine, or an alanine.

An “amino acid derivative,” as used herein, refers to any amino acid, modified amino acid, and/or amino acid analogue, that is not one of the 20 common natural amino acids found in humans. Exemplary amino acid derivatives include natural amino acids not found in humans (e.g., seleno cysteine and pyrrolysine, which may be found in some microorganisms) and unnatural amino acids. Exemplary amino acid derivatives include, but are not limited to, amino acid derivatives commercially available through chemical product manufacturers and distributors (e.g., sigmaaldrich.com/chemistry/chemistry-products.html?TablePage=16274965, accessed on May 6, 2017, which is incorporated herein by reference). One or more amino acid derivative maybe incorporated into a polypeptide at a specific location using translation systems that utilize host cells, orthogonal aminoacyl-tRNA synthetases derived from eubacterial synthetases, orthogonal tRNAs, and an amino acid derivative. For further descriptions, see, e.g., U.S. Pat. No. 9,624,485.

In some embodiments, an EPO polypeptide or other polypeptide described herein comprises an amino acid substitution with an amino acid derivative. In some embodiments, the amino acid derivative is an asparagine derivative, a serine derivative, a threonine derivative, a cysteine, or an alanine derivative.

“Glycosylated,” as used herein, refers to a polypeptide having one or more glycan moieties covalently attached.

A “glycan” or “glycan moiety,” as used herein, refers to monosaccharides linked glycosidically.

Glycans are attached to glycopeptides in several ways, of which N-linked to asparagine and O-linked to serine and threonine are the most relevant for recombinant therapeutic glycoproteins. N-linked glycosylation occurs at the consensus sequence Asn-Xaa-Ser/Thr, where Xaa can be any amino acid except proline.

“Sialylated,” as used herein, refers to a polypeptide having one or more sialyic acid moieties covalently attached.

A variety of approaches for producing glycosylated and sialylated proteins have been developed. See, e.g., Savinova, et al., Applied Biochem & Microbiol. 51(8):827-33 (2015).

“PEGylated,” as used herein, refers to a polypeptide having one or more polyethylene glycol (PEG) moieties associated or covalently or non-covalently attached.

In some embodiments, the EPO polypeptide is glycosylated. In some embodiments, the EPO polypeptide comprises at least one glycan moiety attached to an N-linked glycosylation site. In some embodiments, the EPO polypeptide is sialylated. In some embodiments, the EPO polypeptide is PEGylated. In some embodiments, the EPO polypeptide is PEGylated at a glycan. In some embodiments, the EPO polypeptide is PEGylated at a primary amine. In some embodiments, the EPO polypeptide is PEGylated at the N-terminal alpha-amine. In some embodiments, the EPO polypeptide is glycosylated, sialylated, and/or PEGylated.

### Exemplary Variant IgG Fc Polypeptides

Novel variant IgG Fc polypeptides are provided, for example, variant IgG Fc polypeptides for increased binding to Protein A, for decreased binding to C1q, for decreased binding to CD16, for increased stability, and/or for increased recombinant production.

A “fragment crystallizable polypeptide” or “Fc polypeptide” is the portion of an antibody molecule that interacts with effector molecules and cells. It comprises the C-terminal portions of the immunoglobulin heavy chains. As used herein, an Fc polypeptide includes fragments of the Fc domain having one or more biological activities of an entire Fc polypeptide. In some embodiments, a biological activity of an Fc polypeptide is the ability to bind FcRn. In some embodiments, a biological activity of an Fc polypeptide is the ability to bind C1q. In some embodiments, a biological activity of an Fc polypeptide is the ability to bind CD16. In some embodiments, a biological activity of an Fc polypeptide is the ability to bind protein A. An “effector function” of the Fc polypeptide is an action or activity performed in whole or in part by any antibody in response to a stimulus and may include complement fixation and/or ADCC (antibody-dependent cellular cytotoxicity) induction.

“IgX Fc” refers to an Fc polypeptide derived from a particular antibody isotype (e.g., IgG, IgA, IgD, IgE, IgM, etc.), where “X” denotes the antibody isotype. Thus, “IgG Fc” denotes that the Fc polypeptide is derived from a γ chain, “IgA Fc” denotes that the Fc polypeptide is derived from an a chain, “IgD Fc” denotes that the Fc polypeptide is derived from a 6 chain, “IgE Fc” denotes that the Fc polypeptide is derived from a c chain, “IgM Fc” denotes that the Fc polypeptide is derived from a μ chain, etc. In some embodiments, the IgG Fc polypeptide comprises the hinge, CH2, and CH3, but does not comprise CH1 or CL. In some embodiments, the IgG Fc polypeptide comprises CH2 and CH3, but does not comprise CH1, the hinge, or CL. In some embodiments, the IgG Fc polypeptide comprises CH1, hinge, CH2, CH3, with or without CL. “IgX-N Fc” or “IgGXN Fc” denotes that the Fc polypeptide is derived from a particular subclass of antibody isotype (such as canine IgG subclass IgG-A, IgG-B, IgG-C, or IgG-D; feline IgG subclass IgG1a, IgG1b, or IgG2; or equine IgG subclass IgG1, IgG2, IgG3, IgG4, IgG5, IgG6, or IgG7, etc.), where “N” denotes the subclass.

In some embodiments, an IgX Fc polypeptide or an IgX-N Fc polypeptide is derived from a companion animal, such as a dog, a cat, or a horse. In some embodiments, IgG Fc polypeptides are isolated from canine γ heavy chains, such as IgG-A, IgG-B, IgG-C, or IgG-D. In some instances, IgG Fc polypeptides are isolated from feline γ heavy chains, such as IgG1a, IgG1b, or IgG2. In other instances, IgG Fc polypeptides are isolated from equine γ heavy chains, such as IgG1, IgG2, IgG3, IgG4, IgG5, IgG6, or IgG7.

The terms “IgX Fc” and “IgX Fc polypeptide” include wild-type IgX Fc polypeptides and variant IgX Fc polypeptides, unless indicated otherwise.

“Wild-type” refers to a non-mutated version of a polypeptide that occurs in nature, or a fragment thereof. A wild-type polypeptide may be produced recombinantly.

In some embodiments, a wild-type IgG Fc polypeptide comprises the amino acid sequence of SEQ ID NO: 53, 54, 55, 56, 57, 58, 87, 88, 89, 90, 91, 92, 93, 94, 103, 104, 105, 106, or 107.

A “variant IgG Fc” as used herein refers to an IgG Fc polypeptide that differs from a reference IgG Fc polypeptide by single or multiple amino acid substitutions, deletions, and/or additions and substantially retains at least one biological activity of the reference polypeptide. In some embodiments, a variant (e.g., a variant canine IgG-A Fc, a variant canine IgG-C Fc, a variant canine IgG-D Fc, variant equine IgG2 Fc, variant equine IgG5 Fc, or variant equine IgG6 Fc) has an activity that the reference polypeptide substantially lacks. For example, in some embodiments, a variant canine IgG-A Fc, a variant canine IgG-C Fc, a variant canine IgG-D Fc, variant equine IgG2 Fc, variant equine IgG5 Fc, or variant equine IgG6 Fc binds Protein A.

In some embodiments, a variant IgG Fc polypeptide comprises the amino acid sequence of SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 108, SEQ ID NO: 109, SEQ ID NO: 110, or SEQ ID NO: 111.

Exemplary Variant IgG Fc Polypeptides with Modified Protein A Binding

In some embodiments, a variant IgG Fc polypeptide has modified Protein A binding affinity. In some embodiments, a variant IgG Fc polypeptide has increased binding affinity to Protein A. In some embodiments, a variant IgG Fc polypeptide may be purified using Protein A column chromatography.

In some embodiments, a variant IgG Fc polypeptide comprises an amino acid substitution at a position corresponding to position 21, position 23, position 25, position 80, position 205, and/or position 207 of SEQ ID NO: 54. In some embodiments, a variant IgG Fc polypeptide comprises an amino acid substitution at a position corresponding to position 21, position 23, and/or position 24 of SEQ ID NO: 56. In some embodiments, a variant IgG Fc polypeptide comprises an amino acid substitution at a position corresponding to position 21, position 23, position 25, position 80, and/or position 207 of SEQ ID NO: 58.

In some embodiments, a variant IgG Fc polypeptide comprises an amino acid substitution at a position corresponding to position 15, and/or position 203 of SEQ ID NO: 88. In some embodiments, a variant IgG Fc polypeptide comprises an amino acid substitution at a position corresponding to position 199 and/or position 200 of SEQ ID NO: 92. In some embodiments, a variant IgG Fc polypeptide comprises an amino acid substitution at a position corresponding to position 199, position 200, position 201, and/or 202 of SEQ ID NO: 93.

In some embodiments, a variant IgG Fc polypeptide comprises an amino acid substitution at position 21, position 23, position 25, position 80, position 205, and/or position 207 of SEQ ID NO: 54. In some embodiments, a variant IgG Fc polypeptide comprises an amino acid substitution at position 21, position 23, and/or position 24 of SEQ ID NO: 56 In some embodiments, a variant IgG Fc polypeptide comprises an amino acid substitution at position 21, position 23, position 25, position 80, and/or position 207 of SEQ ID NO: 58.

In some embodiments, a variant IgG Fc polypeptide comprises an amino acid substitution at position 15 and/or position 203 of SEQ ID NO: 88. In some embodiments, a variant IgG Fc polypeptide comprises an amino acid substitution at position 199 and/or position 200 of SEQ ID NO: 92. In some embodiments, a variant IgG Fc polypeptide comprises an amino acid substitution at position 199, position 200, position 201, and/or position 202 of SEQ ID NO: 93.

In some embodiments, a variant IgG Fc polypeptide comprises a threonine at a position corresponding to position 21 of SEQ ID NO: 53, a leucine at a position corresponding to position 23 of SEQ ID NO: 53, an alanine at a position corresponding to position 25 of SEQ ID NO: 53, a glycine at a position corresponding to position 80 of SEQ ID NO: 53, an alanine at a position corresponding to position 205 of SEQ ID NO: 53, and/or a histidine at a position corresponding to position 207 of SEQ ID NO: 53. In some embodiments, a variant IgG Fc polypeptide comprises a threonine at a position corresponding to position 21 of SEQ ID NO: 56, a leucine at a position corresponding to position 23 of SEQ ID NO: 56, and/or an isoleucine at a position corresponding to position 24 of SEQ ID NO: 56. In some embodiments, a variant IgG Fc polypeptide comprises a threonine at a position corresponding to position 21 of SEQ ID NO: 58, a leucine at a position corresponding to position 23 of SEQ ID NO: 58, an alanine at a position corresponding to position 25 of SEQ ID NO: 58, a glycine at a position corresponding to position 80 of SEQ ID NO: 58, and/or a histidine at a position corresponding to position 207 of SEQ ID NO: 58.

In some embodiments, a variant IgG Fc polypeptide comprises a threonine or a valine at a position corresponding to position 15 of SEQ ID NO: 88, and/or a tyrosine or a valine at a position corresponding to position 203 of SEQ ID NO: 88. In some embodiments, a variant IgG Fc polypeptide comprises a leucine at a position corresponding to position 199 of SEQ ID NO: 92, and/or a histidine at a position corresponding to position 200 of SEQ ID NO: 92. In some embodiments, a variant IgG Fc polypeptide comprises an isoleucine at a position corresponding to position 199 of SEQ ID NO: 93, a histidine at a position corresponding to position 200 of SEQ ID NO: 93, an asparagine at a position corresponding to position 201 of SEQ ID NO: 93, and/or a histidine at a position corresponding to position 202 of SEQ ID NO: 93.

In some embodiments, a variant IgG Fc polypeptide comprises a threonine at position 21 of SEQ ID NO: 53, a leucine at position 23 of SEQ ID NO: 53, an alanine at position 25 of SEQ ID NO: 53, a glycine at position 80 of SEQ ID NO: 53, an alanine at position 205 of SEQ ID NO: 53, and/or a histidine at position 207 of SEQ ID NO: 53. In some embodiments, a variant IgG Fc polypeptide comprises a threonine at position 21 of SEQ ID NO: 56, a leucine at position 23 of SEQ ID NO: 56, and/or an isoleucine at position 24 of SEQ ID NO: 56. In some embodiments, a variant IgG Fc polypeptide comprise a threonine at a position 21 of SEQ ID NO: 58, a leucine at position 23 of SEQ ID NO: 58, an alanine at position 25 of SEQ ID NO: 58, a glycine at position 80 of SEQ ID NO: 58, and/or a histidine at position 207 of SEQ ID NO: 58.

In some embodiments, a variant IgG Fc polypeptide comprises a threonine or a valine at position 15 of SEQ ID NO: 88, and/or a tyrosine or a valine at position 203 of SEQ ID NO: 88. In some embodiments, a variant IgG Fc polypeptide comprises a leucine at position 199 of SEQ ID NO: 92, and/or a histidine at position 200 of SEQ ID NO: 92. In some embodiments, a variant IgG Fc polypeptide comprises an isoleucine at position 199 of SEQ ID NO: 93, a histidine at position 200 of SEQ ID NO: 93, an asparagine at position 201 of SEQ ID NO: 93, and/or a histidine at position 202 of SEQ ID NO: 93.

Exemplary Variant IgG Fc Polypeptides with Modified CD16 Binding

In some embodiments, a variant IgG Fc polypeptide has modified CD16 binding affinity. In some embodiments, a variant IgG Fc polypeptide has decreased binding affinity to CD16. In some embodiments, a variant IgG Fc may have a reduced ADCC immune response.

In some embodiments, a variant IgG Fc polypeptide comprises an amino acid substitution at a position corresponding to position 5, position 38, position 39, position 97, and/or position 98 of SEQ ID NO: 54. In some embodiments, a variant IgG Fc polypeptide comprises an amino acid substitution at a position corresponding to position 5, position 38, position 39, position 97, and/or position 98 of SEQ ID NO: 56.

In some embodiments, a variant IgG Fc polypeptide comprises an amino acid substitution at position 5, position 38, position 39, position 97, and/or position 98 of SEQ ID NO: 54. In some embodiments, a variant IgG Fc polypeptide comprises an amino acid substitution at position 5, position 38, position 39, position 97, and/or position 98 of SEQ ID NO: 56.

In some embodiments, a variant IgG Fc polypeptide comprises a proline at a position corresponding to position 5, a glycine at a position corresponding to position 38, an arginine at a position corresponding to position 39, a isoleucine at a position corresponding to position 97, and/or a glycine at a position corresponding to position 98 of SEQ ID NO: 54. In some embodiments, a variant IgG Fc polypeptide comprises a proline at a position corresponding to position 5, a glycine at a position corresponding to position 38, an arginine at a position corresponding to position 39, a isoleucine at a position corresponding to position 97, and/or a glycine at a position corresponding to position 98 of SEQ ID NO: 56.

In some embodiments, a variant IgG Fc polypeptide comprises a proline at position 5, a glycine at position 38, an arginine at position 39, a isoleucine at position 97, and/or a glycine at position 98 of SEQ ID NO: 54. In some embodiments, a variant IgG Fc polypeptide comprises a proline at position 5, a glycine at position 38, an arginine at position 39, a isoleucine at position 97, and/or a glycine at position 98 of SEQ ID NO: 56.

Exemplary Variant IgG Fc Polypeptides with Modified C1q Binding

In some embodiments, a variant IgG Fc polypeptide has modified C1q binding affinity. In some embodiments, a variant IgG Fc polypeptide has reduced binding affinity to C1q. In some embodiments, a variant IgG Fc polypeptide may have reduced complement fixation. In some embodiments, a variant IgG Fc may have a reduced complement-mediated immune response.

In some embodiments, a variant IgG Fc polypeptide comprises an amino acid substitution at a position corresponding to position 93 of SEQ ID NO: 54. In some embodiments, a variant IgG Fc polypeptide comprises an amino acid substitution at a position corresponding to position 93 of SEQ ID NO: 56. In some embodiments, a variant IgG Fc polypeptide comprises an amino acid substitution at a position corresponding to position 87 of SEQ ID NO: 87. In some embodiments, a variant IgG Fc polypeptide comprises an amino acid substitution at a position corresponding to position 87 of SEQ ID NO: 90. In some embodiments, a variant IgG Fc polypeptide comprises an amino acid substitution at a position corresponding to position 87 of SEQ ID NO: 91. In some embodiments, a variant IgG Fc polypeptide comprises an amino acid substitution at a position corresponding to position 87 of SEQ ID NO: 94. In some embodiments, a variant IgG Fc polypeptide comprises an amino acid substitution at a position corresponding to position 198 of SEQ ID NO: 103, of SEQ ID NO: 104, of SEQ ID NO: 105, or of SEQ ID NO: 106.

In some embodiments, a variant IgG Fc polypeptide comprises an amino acid substitution at position 93 of SEQ ID NO: 54. In some embodiments, a variant IgG Fc polypeptide comprises an amino acid substitution at position 93 of SEQ ID NO: 56. In some embodiments, a variant IgG Fc polypeptide comprises an amino acid substitution at position 87 of SEQ ID NO: 87. In some embodiments, a variant IgG Fc polypeptide comprises an amino acid substitution at position 87 of SEQ ID NO: 90. In some embodiments, a variant IgG Fc polypeptide comprises or an amino acid substitution at position 87 of SEQ ID NO: 91. In some embodiments, a variant IgG Fc polypeptide comprises or an amino acid substitution at position 87 of SEQ ID NO: 94. In some embodiments, a variant IgG Fc polypeptide comprises an amino acid substitution at position 198 of SEQ ID NO: 103, of SEQ ID NO: 104, of SEQ ID NO: 105, or of SEQ ID NO: 106.

In some embodiments, a variant IgG Fc polypeptide comprises an arginine at a position corresponding to position 93 of SEQ ID NO: 54. In some embodiments, a variant IgG Fc polypeptide comprises an arginine at a position corresponding to position 93 of SEQ ID NO: 56. In some embodiments, a variant IgG Fc polypeptide comprises a serine at a position corresponding to position 87 of SEQ ID NO: 87. In some embodiments, a variant IgG Fc polypeptide comprises a serine substitution at a position corresponding to position 87 of SEQ ID NO: 90. In some embodiments, a variant IgG Fc polypeptide comprises a serine at a position corresponding to position 87 of SEQ ID NO: 91. In some embodiments, a variant IgG Fc polypeptide comprises a serine at a position corresponding to position 87 of SEQ ID NO: 94. In some embodiments, a variant IgG Fc polypeptide comprises an alanine at a position corresponding to position 198 of SEQ ID NO: 103, of SEQ ID NO: 104, of SEQ ID NO: 105, or of SEQ ID NO: 106.

In some embodiments, a variant IgG Fc polypeptide comprises an arginine at position 93 of SEQ ID NO: 54. In some embodiments, a variant IgG Fc polypeptide comprises an amino acid substitution at position 93 of SEQ ID NO: 56. In some embodiments, a variant IgG Fc polypeptide comprises a serine at position 87 of SEQ ID NO: 87. In some embodiments, a variant IgG Fc polypeptide comprises a serine at position 87 of SEQ ID NO: 90. In some embodiments, a variant IgG Fc polypeptide comprises a serine at position 87 of SEQ ID NO: 91. In some embodiments, a variant IgG Fc polypeptide comprises a serine at position 87 of SEQ ID NO: 94. In some embodiments, a variant IgG Fc polypeptide comprises an alanine at position 198 of SEQ ID NO: 103, of SEQ ID NO: 104, of SEQ ID NO: 105, or of SEQ ID NO: 106.

### Exemplary EPO and EPOR Polypeptide Expression and Production

Polynucleotide sequences that encode all or part of an EPO polypeptide with or without a signal sequence are provided. If a homologous signal sequence (i.e., a signal sequence of wild-type EPO) is not used in the construction of the nucleic acid molecule, then another signal sequence may be used, for example, any one of the signal sequences described in PCT/US06/02951.

Typically, a nucleotide sequence encoding the polypeptide of interest, such as an EPO polypeptide or another polypeptide described herein, is inserted into an expression vector, suitable for expression in a selected host cell.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A vector may be a DNA plasmid deliverable via non-viral methods (e.g., naked DNA, formulated DNA, or liposome), or via viral methods. In some embodiments, the vector is a viral vector, such as a retroviral vector, a herpesviral vector, an adenoviral vector, an adeno-associated viral vector, or a poxviral vector. The vector may be a bacterial vector.

The term “expression system,” as used herein, refers to a combination of an expression vector and at least one additional vector. The combination may be deliverable via non-viral or via viral methods.

In some embodiments, the expression system comprises an expression vector and a vector comprising a regulatory sequence (e.g., a nucleic acid sequence encoding a transcription factor or microRNA).

Expression of an EPO or EPOR polypeptide described herein may be regulated to prevent excessive production of EPO or EPOR in vivo. Controlled expression may reduce immunogenicity, polycythemia (over production of red blood cells), or other negative effects. There are many known methods of controlling gene regulation in vitro and in vivo, such as tetracycline responsive systems, micro RNA regulated systems, or hypoxia-inducible systems (e.g., use of prolyl hydroxylase to activate hypoxia-inducible promoters or enhancers).

The term “regulatory sequence” (also referred to as a “regulatory region” or “regulatory element”) refers to a nucleic acid sequence that facilitates and/or controls gene expression and/or protein expression, either directly or indirectly. A regulatory sequence may be a promoter, enhancer, silencer, or a nucleic acid sequence encoding a micro RNA (miRNA) or transcription factor. Regulatory sequences may increase or decrease gene expression and/or protein expression.

In some embodiments, a regulatory sequence binds regulatory proteins, such as transcription factors, to control gene expression and/or protein expression. In some embodiments, a regulatory sequence encodes a transcription factor that controls gene expression and/or protein expression. In some embodiments, a regulatory sequence encodes a miRNA that binds to a target mRNA to control protein expression.

In some embodiments, the regulatory sequence is a controllable regulatory sequence. In some embodiments, the regulatory sequence is an uncontrollable regulatory sequence, such as a constitutive promoter (e.g., a CMV promoter). In some embodiments, the regulatory sequence is a positive regulatory sequence, such as a promoter. In some embodiments, the regulatory sequence is a negative regulatory sequence, such as a silencer. In some embodiments, the regulatory sequence provides for transient, inducible (e.g., tetracycline-responsive promoter, or hypoxia-inducible promoter), and/or tissue-specific gene expression and/or protein expression.

In some embodiments, the regulatory sequence is operably linked to the nucleic acids encoding the EPO polypeptides (coding sequence) of the present disclosure. The regulatory sequence need not be contiguous with the coding sequence as long as they function to direct the expression of the encoded polypeptides. Thus, for example, intervening untranslated yet transcribed sequences may be present between a promoter sequence and a coding sequence and the promoter sequence may still be considered “operably linked” to the coding sequence.

In some embodiments, the regulatory sequence is not operably linked to the nucleic acids encoding the EPO polypeptides of the present disclosure. For example, the regulatory sequence may be a microRNA sequence or transcription factor expressed from the same vector or a different vector as the nucleic acids encoding the EPO polypeptides.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 cells, and CHO cells, and their derivatives, such as 293-6E, DG-44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.”

In some embodiments, the EPO polypeptide or another polypeptide described herein is isolated using chromatography, such as size exclusion chromatography, ion exchange chromatography, protein A column chromatography, hydrophobic interaction chromatography, CHT chromatography, and/or synthetic molecule conjugated resin chromatography (e.g., His tag affinity column chromatography). In some embodiments, the EPO polypeptide or another polypeptide described herein is isolated using Capto Butyl column chromatography, cation-exchange column chromatography, anion-exchange column chromatography, and/or mixed-mode column chromatography. In some embodiments, the EPO polypeptide or another polypeptide described herein is isolated using a combination of chromatography methods and/or columns.

In some embodiments, the method of production or isolation further comprises inactivating or removing any viruses.

The term “isoelectric point” or “pI,” as used herein refers to the pH at which a molecule carries no net electrical charge and/or does not migrate further in an electric field, as determined by isoelectric focusing.

The term “range of isoelectric points,” as used herein refers to the range of pHs at which a plurality of molecules carries no net electrical charge and/or do not migrate further in an electric field, as determined by isoelectric focusing.

In some embodiments, a composition comprises EPO polypeptides having a range of isoelectric points of from about 1 to about 3.5, of from about 1.5 to about 3.5, of from about 2 to about 3.5, of from about 2.5 to about 3.5, of from about 3 to about 3.5, of about 3.5 or less, or of about 3 or less, as determined by isoelectric focusing. In some embodiments, a composition comprises an acidic fraction of EPO polypeptides having a range of isoelectric points of from about 1 to about 3.5, of from about 1.5 to about 3.5, of from about 2 to about 3.5, of from about 2.5 to about 3.5, of from about 3 to about 3.5, of about 3.5 or less, or of about 3 or less, as determined by isoelectric focusing. In some embodiments, a composition comprises a high sialylation fraction of EPO polypeptides having a range of isoelectric points of from about 1 to about 3.5, of from about 1.5 to about 3.5, of from about 2 to about 3.5, of from about 2.5 to about 3.5, of from about 3 to about 3.5, of about 3.5 or less, or of about 3 or less, as determined by isoelectric focusing.

In some embodiments, a composition comprises EPO polypeptides having a range of isoelectric points of from about 3.5 to about 6, of from about 4 to about 6, of from about 4.5 to about 6, of from about 5 to about 6, of from about 5.5 to about 6, of from about 3.5 to about 5, of from about 4 to about 5, of from about 4.5 to about 5, of about 3.5 or greater, of about 4 or greater, or of about 4.5 or greater, as determined by isoelectric focusing. In some embodiments, a composition comprises a basic fraction of EPO polypeptides having a range of isoelectric points of from about 3.5 to about 6, of from about 4 to about 6, of from about 4.5 to about 6, of from about 5 to about 6, of from about 5.5 to about 6, of from about 3.5 to about 5, of from about 4 to about 5, of from about 4.5 to about 5, of about 3.5 or greater, of about 4 or greater, or of about 4.5 or greater, as determined by isoelectric focusing. In some embodiments, a composition comprises a low sialylation fraction of EPO polypeptides having a range of isoelectric points of from about 3.5 to about 6, of from about 4 to about 6, of from about 4.5 to about 6, of from about 5 to about 6, of from about 5.5 to about 6, of from about 3.5 to about 5, of from about 4 to about 5, of from about 4.5 to about 5, of about 3.5 or greater, of about 4 or greater, or of about 4.5 or greater, as determined by isoelectric focusing.

### Exemplary EPO Polypeptide Affinity to EPOR

The term “affinity” means the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art, such as, for example, immunoblot, ELISA KD, KinEx A, biolayer interferometry (BLI), or surface plasmon resonance devices.

The terms “K_{D},” “K_{d},” “Kd” or “Kd value” as used interchangeably to refer to the equilibrium dissociation constant of an antibody-antigen interaction. In some embodiments, the K_{d} of the antibody is measured by using biolayer interferometry assays using a biosensor, such as an Octet® System (Pall ForteBio LLC, Fremont, Calif.) according to the supplier's instructions. Briefly, biotinylated antigen is bound to the sensor tip and the association of antibody is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{d}) is calculated as the ratio of k_{off}/kₒₙ. The term “kₒₙ” refers to the rate constant for association of an antibody to an antigen and the term “k_{off}” refers to the rate constant for dissociation of an antibody from the antibody/antigen complex.

The term “binds” to a ligand or receptor is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time.

To “increase” or “stimulate” means to increase, improve, or augment an activity, function, or amount as compared to a reference. In some embodiments, by “reduce” or “inhibit” is meant the ability to cause an overall increase of 20% or greater. In some embodiments, by “increase” or “stimulate” is meant the ability to cause an overall increase of 50% or greater. In some embodiments, by “increase” or “stimulate” is meant the ability to cause an overall increase of 75%, 85%, 90%, 95%, or greater. In some embodiments, the amount noted above is stimulated or increased over a period of time, relative to a control dose (such as a placebo) over the same period of time.

A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

In some embodiments, administration of an EPO polypeptide or nucleic acid of the present invention may result in an increase of the hematocrit percent to increases to at least 25%, or at least 26%, or at least 27%, or at least 28%, or at least 29%, or at least 30%, or at least 32%, or at least 35%, or at least 38%, or at least 40%, or at least 42%, or at least 45%, or at least 48%.

### Exemplary Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

In some embodiments, the pharmaceutically acceptable carrier has a pH of from about 6.2 to about 7, of from about 6 to about 7.2, of from about 6.4 to about 6.8, of about 6, or of about 7 and comprises sodium phosphate and sodium chloride. In some embodiments, the pharmaceutically acceptable carrier has a pH of from about 6.2 to about 7, of from about 6 to about 7.2, of about 6, of from about 6.4 to about 6.8, or of about 7 and comprises sodium citrate and sodium chloride.

In some embodiments, the pharmaceutically acceptable carrier comprises sodium phosphate, sodium chloride, and polysorbate 80. In some embodiments, the pharmaceutically acceptable carrier comprises sodium phosphate, sodium chloride, and polysorbate 20. In some embodiments, the pharmaceutically acceptable carrier comprises sodium citrate, sodium chloride, and polysorbate 20. In some embodiments, the pharmaceutically acceptable carrier comprises sodium citrate, sodium chloride, and polysorbate 80.

In some embodiments, the pharmaceutically acceptable carrier comprises sodium chloride at a concentration of from about 100 nM to about 180 nM, of from about 110 nM to about 170 nM, of from about 120 nM to about 160 nM, of from about 130 nM to about 150 nM, of about 140 nM, of from about 130 nM to about 160 nM, of from about 120 nM to about 150 nM, of about 100 nM, of about 110 nM, of about 120 nM, of about 130 nM, of about 140 nM, of about 150 nM, of about 160 nM, of about 170 nM, or of about 180 nM.

In some embodiments, the pharmaceutically acceptable carrier comprises sodium phosphate at a concentration of from about 100 nM to about 180 nM, of from about 110 nM to about 170 nM, of from about 120 nM to about 160 nM, of from about 130 nM to about 150 nM, of about 140 nM, of from about 130 nM to about 160 nM, of from about 120 nM to about 150 nM, of about 100 nM, of about 110 nM, of about 120 nM, of about 130 nM, of about 140 nM, of about 150 nM, of about 160 nM, of about 170 nM, or of about 180 nM.

In some embodiments, the pharmaceutically acceptable carrier comprises a polysorbate at a concentration of about 550 nM to about 750 nM, of about 570 nM to about 730 nM, of about 590 nM to about 720 nM, of about 600 nM to about 700 nM, of about, 620 nM to about 680 nM, of about 640 nM to about 660 nM, of about 650 nM, of about 570 nM to about 670 nM, of about 550 nM to about 650 nM, of about 650 nM to about 750 nM, of about 630 nm to about 700 nM, or of about 670 nM to about 600 nM. In some embodiments, the polysorbate is polysorbate 80. In some embodiments, the polysorbate is polysorbate 20.

In some embodiments, the pharmaceutically acceptable carrier comprises m-cresol or benzyl alcohol. In some embodiments, the concentration of m-cresol is about 0.2%, of from about 0.1% to about 0.3%, of from about 0.08% to about 0.25%, or of from about 0.05% to about 0.25%. In some embodiments, the concentration of benzyl alcohol is about 1%, of from about 0.5% to about 2%, of from about 0.2% to about 2.5%, of about 1% to about 5%, of about 0.5% to about 5%, or of about 1% to about 3%.

The pharmaceutical composition can be stored in lyophilized form; thus, in some embodiments, the preparation process includes a lyophilization step. The lyophilized composition is then reformulated, typically as an aqueous composition suitable for parenteral administration, prior to administration to the companion animal. In other embodiments, particularly where the protein is highly stable to thermal and oxidative denaturation, the pharmaceutical composition can be stored as a liquid, i.e., aqueous, composition, which may be administered directly, or with appropriate dilution, to the dog, cat, or horse. It can be reconstituted with sterile Water for Injection (WFI), and Bacteriostatic reagents such benzyl alcohol may be included. Thus, the invention provides pharmaceutical compositions in both solid and liquid form.

The pH of the pharmaceutical compositions typically will be in the range of from about pH 6 to pH 8 when administered, for example about 6, about 6.2, about 6.4, about 6.6, about 6.8, about 7, about 7.2. The formulations of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

The pharmaceutical formulations of the invention are useful in the methods of the invention for treating anemia associated conditions in companion animals, such as cats. For example, the methods described herein include administering a therapeutically effective dose of a nucleic acid or polypeptide of the disclosure to a companion animal. In many embodiments, the therapeutically effective dose is administered parenterally, for example by subcutaneous administration, intravenous infusion, intravenous bolus injection, or intramuscular injection.

Thus, in accordance with the methods of the invention, an EPO polypeptide or nucleic acid, other polypeptide or nucleic acid of the present invention, or a pharmaceutical composition is administered in a therapeutically effective dose to a feline, canine, equine, or human.

In some embodiments, the therapeutically effective dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

### Exemplary Uses of EPO and EPOR ECD Polypeptides

The EPO polypeptides comprising one or more additional N-glycosylation site(s) or cysteine residues or pharmaceutical compositions comprising the EPO polypeptides disclosed herein may be useful for treating non-regenerative anemia. A non-regenerative anemia condition may be exhibited in a companion animal, including, but not limited to, canine, feline, or equine.

The polypeptides comprising an extracellular domain of EPOR or pharmaceutical compositions comprising the EPOR ECD polypeptides disclosed herein may be useful for treating polycythemia.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also, encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, an EPO polypeptide, nucleic acid, vector, expression system, or pharmaceutical compositions comprising it can be utilized in accordance with the methods herein to treat EPO deficient or EPO insensitivity-induced conditions. In some embodiments, an EPO polypeptide, nucleic acid, vector, expression system or pharmaceutical composition is administered to a companion animal, such as a canine, a feline, or equine, to treat EPO deficient or EPO insensitivity-induced conditions. In some embodiments, an EPO polypeptide, nucleic acid, vector, expression system, or pharmaceutical compositions is administered to a companion animal, such as a canine, a feline, or equine, to treat anemia.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, an EPO or EPOR polypeptide, nucleic acid, vector, or expression system or pharmaceutical composition is administered parenterally, by subcutaneous administration, intravenous infusion, or intramuscular injection. In some embodiments, an EPO or EPOR polypeptide, nucleic acid, vector, expression system, or pharmaceutical composition is administered as a bolus injection or by continuous infusion over a period of time. In some embodiments, an EPO or EPOR polypeptide, nucleic acid, vector, expression system, or pharmaceutical composition is administered by an intramuscular, an intraperitoneal, an intracerebrospinal, a subcutaneous, an intra-arterial, an intrasynovial, an intrathecal, or an inhalation route.

An EPO or EPOR polypeptide described herein may be administered in an amount in the range of 0.0001 mg/kg body weight to 100 mg/kg body weight per dose. In some embodiments, an EPO or EPOR polypeptide may be administered in an amount in the range of 0.0005 mg/kg body weight to 50 mg/kg body weight per dose. In some embodiments, an EPO polypeptide may be administered in an amount in the range of 0.001 mg/kg body weight to 10 mg/kg body weight per dose. In some embodiments, an EPO or EPOR polypeptide may be administered in an amount in the range of from about 1 μg/kg body weight to about 10 μg/kg body weight, or about 1 μg/kg body weight to about 5 μg/kg body weight, or about 1 μg/kg body weight, or about 3 μg/kg body weight, or about 5 μg/kg body weight, or about 10 μg/kg body weight.

An EPO or EPOR polypeptide, nucleic acid, vector, expression system, or a pharmaceutical composition can be administered to a companion animal at one time or over a series of treatments. For example, an EPO or EPOR polypeptide, nucleic acid, vector, expression system, or pharmaceutical composition may be administered at least once, more than once, at least twice, at least three times, at least four times, or at least five times, or chronically use.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

Provided herein are methods of using the EPO polypeptides and polynucleotides for detection, diagnosis and monitoring of an anemia condition. For example, anemia may be detected, diagnosed, or monitored by measuring hematocrit percentage (HCT %) using standard methods. Provided herein are methods of determining whether a companion animal will respond to EPO polypeptide. In some embodiments, the method comprises detecting whether the animal has cells that express EPOR using an EPO polypeptide. In some embodiments, the method of detection comprises contacting the sample with an EPO polypeptide or polynucleotide and determining whether the level of binding differs from that of a reference or comparison sample (such as a control). In some embodiments, the method may be useful to determine whether the antibodies or polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

Various methods known in the art for detecting specific ligand-receptor binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or p-galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including EPO or EPOR can be labeled with a detectable moiety including but not limited to radioisotopes, fluorescent labels, and various enzyme-substrate labels know in the art. Methods of conjugating labels to a protein are known in the art.

The following examples illustrate particular aspects of the disclosure and are not intended in any way to limit the disclosure.

### EXAMPLES

### Example 1

### Identification of N-Linked Glycosylation Sites for Canine EPO

One approach for generating long acting canine EPO polypeptides is by introducing additional glycosylation site(s). Wild-type canine EPO has three N-linked glycosylation sites—at amino acid positions 50-52, 64-66, and 109-111 of wild-type canine EPO precursor form (SEQ ID NO: 1 or “wild-type canine EPO”).

Additional N-linked glycosylation sites may be introduced into wild-type canine EPO amino acid sequences. For example, one, two, three, four, five, or six additional N-linked glycosylation sites may be introduced into wild-type canine EPO amino acid sequences. The N-linked glycosylation site may have a consensus sequence of Asn-Xaa-Ser/Thr, where Xaa is any amino acid except proline. Addition of one or more glycosylation sites may increase the molecular size of a canine EPO molecule, provide more sialylation sites, provide sites for glycoconjugation, such as pegylation, and/or improve the half-life of the molecule in an animal's serum.

Table 6 lists amino acid substitutions of wild-type canine EPO that may be used to generate one or more additional N-linked glycosylation sites.

TABLE 6

| | Amino acid substitutions for N-linked glycosylation sites | |
|---|---|---|
| | Based on wt canine EPO | Based on wt canine EPO |
| Analog No. | sequence (SEQ ID NO: 1) | sequence (SEQ ID NO: 2) |
| 1 | N47S49 | N21S23 |
| 2 | N47T49 | N21T23 |
| 3 | N55S57 | N29S31 |
| 4 | N55T57 | N29T31 |
| 5 | N56S58 | N30S32 |
| 6 | N56T58 | N30T32 |
| 7 | N60 | N34 |
| 8 | N60T62 | N34T36 |
| 9 | N61S63 | N35S37 |
| 10 | N61T63 | N35T37 |
| 11 | N79S81 | N53S55 |
| 12 | N79T81 | N53T55 |
| 13 | N81S83 | N55S57 |
| 14 | N81T83 | N55T57 |
| 15 | N82S84 | N56S58 |
| 16 | N82T84 | N56T58 |
| 17 | N91S93 | N65S67 |
| 18 | N91T93 | N65T67 |
| 19 | N92S94 | N66S68 |
| 20 | N92T94 | N66T68 |
| 21 | N97S99 | N71S73 |
| 22 | N97T99 | N71T73 |
| 23 | N98S100 | N72S74 |
| 24 | N98T100 | N72T74 |
| 25 | N99S101 | N73S75 |
| 26 | N99T101 | N73T75 |
| 27 | N112*X113 | N86*X87 |
| 28 | N112*X113T114 | N86*X87T88 |
| 29 | N113S115 | N87S89 |
| 30 | N113T115 | N87T89 |
| 31 | *X113N114S116 | *X87N88S90 |
| 32 | *X113N114 | *X87N88 |
| 33 | N115S117 | N89S91 |
| 34 | N115T117 | N89T91 |
| 35 | N116*X117S118 | N90*X91S92 |
| 36 | N116*X117T118 | N90*X91T92 |
| 37 | N137S139 | N111S113 |
| 38 | N137T139 | N111T113 |
| 39 | N138S140 | N112S114 |
| 40 | N138T140 | N112T114 |
| 41 | N140S142 | N114S116 |
| 42 | N140T142 | N114T116 |
| 43 | N141S143 | N115S117 |
| 44 | N141T143 | N115T117 |
| 45 | N142S144 | N116S118 |
| 46 | N142T144 | N116T118 |
| 47 | N143S145 | N117S119 |
| 48 | N143T145 | N117T119 |
| 49 | N144 | N118 |
| 50 | N144T146 | N118T120 |
| 51 | N145S147 | N119S121 |
| 52 | N145T147 | N119T121 |
| 53 | N146S148 | N120S122 |
| 54 | N146T148 | N120T122 |
| 55 | N147*X148S149 | N121*X122S123 |
| 56 | N147*X148T149 | N121*X122T123 |
| 57 | N148S150 | N122S124 |
| 58 | N148T150 | N122T124 |
| 59 | N149S151 | N123S125 |
| 60 | N149T151 | N123T125 |
| 71 | *X148N149S151 | *X122N123S125 |
| 72 | *X148N149T151 | *X122N123T125 |
| 61 | N150 | N124 |
| 62 | N150T152 | N124T126 |
| 63 | N161S163 | N135S137 |
| 64 | N161 | N135 |
| 65 | N162S164 | N136S138 |
| 66 | N162T164 | N136T138 |
| 67 | N184S186 | N158S160 |
| 68 | N184T186 | N158T160 |
| 69 | N186S188 | N162S164 |
| 70 | N186T188 | N162T164 |

*X indicates any amino acid except proline (such as E, V, S, A, etc.).

### Example 2

### Identification of N-Linked Glycosylation Sites for Equine EPO

Long acting equine EPO polypeptides may also be prepared by introducing additional glycosylation site(s). Wild-type equine EPO has three N-linked glycosylation sites—at amino acid positions 50-52, 64-66, and 109-111 of wild-type equine EPO precursor form (SEQ ID NO: 3).

Additional N-linked glycosylation sites may be introduced into wild-type equine EPO amino acid sequences. For example, one, two, three, four, five, or six additional N-linked glycosylation sites may be introduced into wild-type equine EPO amino acid sequences. The N-linked glycosylation site may have a consensus sequence of Asn-Xaa-Ser/Thr, where Xaa is any amino acid except proline. Addition of one or more glycosylation sites may increase the molecular size of an equine EPO molecule, provide more sialylation sites, provide sites for glycoconjugation, such as pegylation, and/or improve the half-life of the molecule in an animal's serum.

Table 7 lists amino acid substitutions of wild-type equine EPO that may be used to generate one or more additional N-linked glycosylation sites.

TABLE 7

| | Amino acid substitutions for N-linked glycosylation sites | |
|---|---|---|
| | Based on wt equine EPO | Based on wt equine EPO |
| Analog No. | sequence (SEQ ID NO: 3) | sequence (SEQ ID NO: 4) |
| 1 | N47S49 | N21S23 |
| 2 | N47T49 | N21T23 |
| 3 | N55S57 | N29S31 |
| 4 | N55T57 | N29T31 |
| 5 | N56S58 | N30S32 |
| 6 | N56T58 | N30T32 |
| 7 | N60S62 | N34S36 |
| 8 | N60T62 | N34T36 |
| 9 | N61S63 | N35S37 |
| 10 | N61T63 | N35T37 |
| 11 | N79S81 | N53S55 |
| 12 | N79T81 | N53T55 |
| 13 | N81S83 | N55S57 |
| 14 | N81T83 | N55T57 |
| 15 | N82S84 | N56S58 |
| 16 | N82T84 | N56T58 |
| 17 | N91S93 | N65S67 |
| 18 | N91T93 | N65T67 |
| 19 | N92S94 | N66S68 |
| 20 | N92T94 | N66T68 |
| 21 | N97S99 | N71S73 |
| 22 | N97T99 | N71T73 |
| 23 | N98S100 | N72S74 |
| 24 | N98T100 | N72T74 |
| 25 | N99S101 | N73S75 |
| 26 | N99T101 | N73T75 |
| 27 | N112*X113 | N86*X87 |
| 28 | N112*X113T114 | N86*X87T88 |
| 29 | N113S115 | N87S89 |
| 30 | N113T115 | N87T89 |
| 31 | *X113N114S116 | *X87N88S90 |
| 32 | *X113N114 | *X87N88 |
| 33 | N115S117 | N89S91 |
| 34 | N115T117 | N89T91 |
| 35 | N116S118 | N90S92 |
| 36 | N116T118 | N90T92 |
| 37 | N137S139 | N111S113 |
| 38 | N137T139 | N111T113 |
| 39 | N138S140 | N112S114 |
| 40 | N138T140 | N112T114 |
| 41 | N140S142 | N114S116 |
| 42 | N140T142 | N114T116 |
| 43 | N141S143 | N115S117 |
| 44 | N141T143 | N115T117 |
| 45 | N142S144 | N116S118 |
| 46 | N142T144 | N116T118 |
| 47 | N143S145 | N117S119 |
| 48 | N143T145 | N117T119 |
| 49 | N144 | N118 |
| 50 | N144T146 | N118T120 |
| 51 | N145S147 | N119S121 |
| 52 | N145T147 | N119T121 |
| 53 | N146*X147S148 | N120*X121S122 |
| 54 | N146*X147T148 | N120*X121T122 |
| 55 | N147*X148S149 | N121*X122S123 |
| 56 | N147*X148T149 | N121*X122T123 |
| 57 | N148S150 | N122S124 |
| 58 | N148T150 | N122T124 |
| 59 | N149S151 | N123S125 |
| 60 | N149T151 | N123T125 |
| 71 | *X148N149S151 | *X122N123S125 |
| 72 | *X148N149T151 | *X122N123T125 |
| 61 | N150 | N124 |
| 62 | N150T152 | N124T126 |
| 63 | N161S163 | N135S137 |
| 64 | N161 | N135 |
| 65 | N162S164 | N136S138 |
| 66 | N162T164 | N136T138 |
| 67 | N184S186 | N158S160 |
| 68 | N184T186 | N158T160 |
| 69 | N186S188 | N162S164 |
| 70 | N186T188 | N162T164 |

*X indicates any amino acid except proline (such as E, V, S, A, etc.).

### Example 3

### Expression of EPO with Additional N-Linked Glycosylation Sites

The nucleotide sequence encoding a EPO polypeptide having additional N-linked glycosylation sites may be inserted into an expression vector and transfected into CHO host cells. The CHO cells are selected for high yield and stability of expression of the EPO polypeptide, for example by using a DHFR gene on the expression vector and methotrexate-mediated gene amplification, as is known in the art.

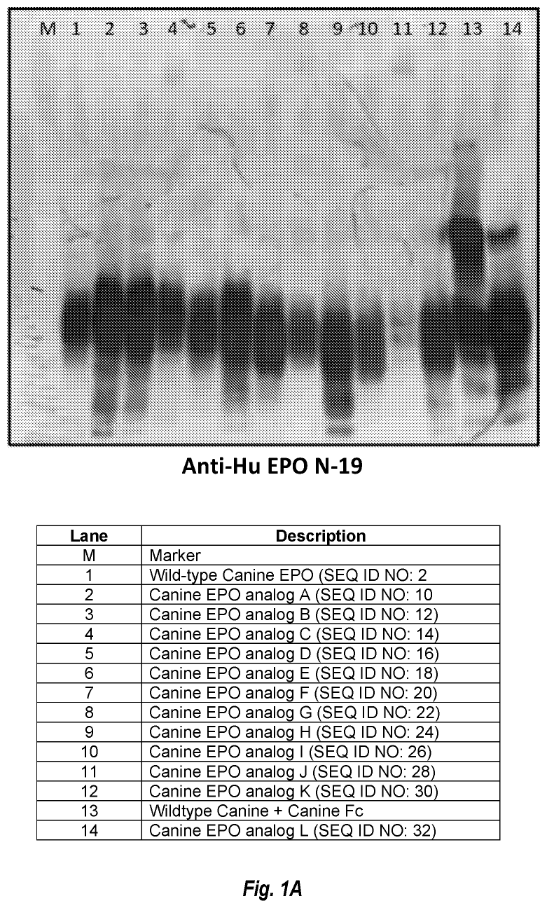

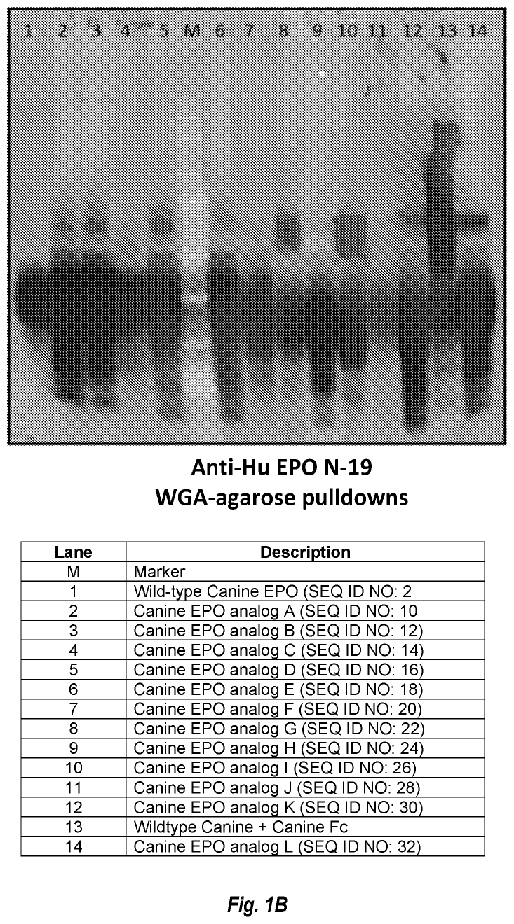

For example, nucleotide sequences encoding various canine EPO analogs having one or more additional N-linked glycosylation sites compared to wild-type canine EPO were chemically synthesized. Wild-type canine EPO and exemplary canine EPO analogs listed in Table 8 (below) were transiently expressed in HEK293 cells and visualized by Western blot using anti-human EPO N-19 antibody (FIGS. 1A and 1B, lane 1 (wild-type canine EPO; SEQ ID NO: 2) and lanes 2-7 (canine EPO analogs; SEQ ID NOs: 10, 12, 14, 16, 18, 20)).

TABLE 8

| | | | Amino acid substitutions |
|---|---|---|---|
| FIG. 1A & 1B | | SEQ ID | based on wt canine |
| Lane No. | Analog ID | NO: | EPO mature (SEQ ID NO: 2) |
| 1 | Wild-type | 2 | None |
| 2 | A | 10 | A30N; G32T; P87E; S88N |
| 3 | B | 12 | A30N; G32T; P87V; S88N |
| 4 | C | 14 | D55N; G57T |
| 5 | D | 16 | L112N; A114T |
| 6 | E | 18 | L121N; P122V; E123T |
| 7 | F | 20 | P122E; E123N; A125T |

### Example 4

### EPO Intramolecular Disulfides

Wild-type canine EPO has two cysteine pairs for forming disulfide bonds. To further increase stability of EPO polypeptides, suitable positions for additional intramolecular disulfide binding were identified by three-dimensional protein modeling and analysis. Additional disulfide binding may prevent EPO from unfolding and enhance protease resistance leading to enhanced product shelf-life stability and enhanced in vivo pharmacokinetics.

Additional cysteines may be incorporated into canine, equine, and feline EPO polypeptides at position(s) 19, 22, 23, 42, 60, 64, 66, 94, 117, 118, and/or 146 of the mature EPO sequence (SEQ ID NO: 2, SEQ ID NO: 4, or SEQ ID NO: 8), which correspond to position(s) 45, 48, 49, 68, 86, 90, 92 120, 143, 144, and/or 172 of the precursor EPO sequence (SEQ ID NO: 1, SEQ ID NO: 3, or SEQ ID NO: 7).

The additional cysteine(s) may be incorporated into canine, equine, and feline EPO polypeptides as one or more pairs at positions 19 and 146, positions 22 and 94, positions 23 and 146, positions 42 and 66, positions 60 and 117, and positions 64 and 118 of the mature EPO sequence (SEQ ID NO: 2, SEQ ID NO: 4, and SEQ ID NO: 8), which correspond to positions 45 and 172, 48 and 120, 49 and 172, 68 and 92, 90 and 144, and 86 and 143 of the precursor EPO sequence (SEQ ID NO: 1, SEQ ID NO: 3, and SEQ ID NO: 7).

For example, nucleotide sequences encoding various canine EPO analogs having an additional cysteine pair compared to wild-type canine EPO were chemically synthesized. Wild-type canine EPO and exemplary canine EPO analogs listed in Table 9 (below) were transiently expressed in HEK293 cells and visualized by Western blot using anti-human EPO N-19 antibody (FIGS. 1A and 1B, lane 1 (wild-type canine EPO; SEQ ID NO: 2) and lanes 8-12 and 14 (canine EPO analogs; SEQ ID NOs 22, 24, 26, 28, 30, and 32)).

TABLE 9

| | | | Cysteine substitutions |
|---|---|---|---|
| FIG. 1A & 1B | | SEQ ID | based on wt canine |
| Lane No. | Analog ID | NO: | EPO mature (SEQ ID NO: 2) |
| 1 | Wild-type | 2 | None |
| 8 | G | 22 | A19C; S146C |
| 9 | H | 24 | A22C; H94C |
| 10 | I | 26 | E23C; S146C |
| 11 | J | 28 | P42C; G66C |
| 12 | K | 30 | W64C; A118C |
| 14 | L | 32 | A60C; E117C |

### Example 5

### Isolation of EPO Polypeptides

Cell lines expressing EPO polypeptides may be cultured until sufficient quantities of the EPO polypeptide are produced. The polypeptide may be isolated by one or more of various steps, including Capto Butyl column chromatography, cation-exchange (CEX) column chromatography, anion-exchange (AEX) column chromatography, or other chromatographic methods. Other chromatographic methods may include ion exchange column chromatography, hydrophobic interaction column chromatography, mixed mode column chromatography (e.g., CHT and/or ultimodal mode column chromatography, such as CaptoMMC). Low pH or other viral inactivation and viral removal steps may be applied. The isolated EPO polypeptide may be admixed with excipients, and sterilized by filtration to prepare a pharmaceutical composition of the invention. The pharmaceutical composition may be administered to a companion animal with anemia in a dose sufficient to stimulate hematopoietic activity.

When cell viabilities dropped below 95%, the supernatant may be harvested by clarifying the conditioned media. For example, a combination of chromatography steps may be used to purify EPO polypeptides. Media from CHO cells expressing the EPO polypeptide may be collected and conditioned with the addition of sodium chloride (NaCl) such that the media would have an NaCl concentration of greater than 1 M NaCl so that the EPO polypeptide can bind to a Capto Butyl column (GE Healthcare Life Sciences) by hydrophobic interaction chromatography (HIC). EPO is understood to bind to a Capto Butyl column at a pH of about 5.75 to about 8.5 with about 1 to about 2.5 M NaCl. The conditioned media may be clarified by centrifugation and filtration and loaded onto the Capto Butyl column. Bound EPO polypeptide may be eluted from the column with 30% isopropanol at a pH of about 5.6.

The host cell proteins fractionated away can be analyzed using CHO host cell protein analysis ELISA kit (Catalog No. CM015; Cygnus Technologies). At least about 95% of host cell proteins may be fractionated away from EPO proteins by this purification method.

The eluate from the Capto Butyl column may be loaded directly onto an SP cation-exchange (CEX) column (GE Healthcare Life Sciences) as a subtraction chromatography step. Under this loading condition of 20-40% isopropanol at a pH of about 5.6, EPO polypeptides flow through the SP CEX column while host cell proteins should bind.

The flow-through from the SP CEX column may be loaded directly onto a Capto Q anion-exchange (AEX) column (GE Healthcare Life Sciences), which binds EPO polypeptides in 30% isopropanol at a pH of about 5.6±0.5. A pH 4 wash may be added to remove a fraction of basic EPO polypeptides while a fraction of acidic EPO polypeptides remains with the solid phase. The EPO polypeptide acidic fraction may be eluted with 0.15 M NaCl at pH 4 and the eluate kept at pH 4 for greater than 90 minutes at ambient temperature to inactivate viruses. This step also increases the concentration of the EPO polypeptide acidic fraction.

The eluate containing the EPO polypeptide acidic fraction may be loaded directly onto an SP CEX column (GE Healthcare Life Sciences) to fractionate away any residual endotoxin and basic EPO polypeptide fraction, along with further concentrating the EPO polypeptide acidic fraction. The EPO polypeptide acidic fraction may be eluted with 0.5 M NaCl at pH 4 and the eluate kept at pH 4 for greater than 90 minutes at ambient temperature to inactivate viruses.

Tangential flow filtration (TFF) may be used to concentrate the acidic and basic fractions EPO polypeptide fractions. A gel filtration step using Sperdex200 may be performed to remove any aggregates and as a buffer exchange to the desired buffer (e.g. a formulation buffer as described below). A nanofiltration step may be performed to remove any residual viral contaminants.

### Example 6

### EPO Buffer Formulations

Thermostability of feline EPO in various buffer formulations was analyzed. Buffers containing 20 mM sodium citrate or 20 mM sodium phosphate at pH 6.2 and pH 7 were considered. Sodium chloride at a final concentration of 140 mM was used in all buffers. Polysorbate 80 and 20 were compared. Bacteriostatic reagents benzyl alcohol and m-cresol were also compared. The melting temperature (Tm) of a feline EPO analog at a concentration of 6 μg/μL in each buffer was measured by differential scanning fluorescence technique from 20° C. to 95° C. Table 10 lists Tm values of the feline EPO analog in the various buffers tested. The thermostability of other EPO polypeptides in the various buffers may be similarly analyzed.

TABLE 10

| Formulation | | Melting temperature |
|---|---|---|
| Designation | Buffer Formulation | (Tm ° C.) |
| A1 | 20 mM sodium citrate | 55 |
| | 140 mM sodium chloride | |
| | pH 6.2 | |
| A2 | A1 + | 54 |
| | 650 nM polysorbate 80 | |
| A3 | A1 + | 52 |
| | 650 nM polysorbate 20 | |
| A4 | A2 + | 42 |
| | 1% benzyl alcohol | |
| A5 | A2 + | 50 |
| | 0.2% m-cresol | |
| A6 | A3 + | no peak* |
| | 1% benzyl alcohol | |
| A7 | A3 + | 35 |
| | 0.2% m-cresol | |
| Bl | 20 mM sodium citrate | 50 |
| | 140 mM sodium chloride | |
| | pH 7 | |
| B2 | B1 + | 51 |
| | 650 nM polysorbate 80 | |
| B3 | B1 + | 50 |
| | 650 nM polysorbate 20 | |
| B4 | B2 + | no peak* |
| | 1% benzyl alcohol | |
| B5 | B2 + | 45 |
| | 0.2% m-cresol | |
| B6 | B3 + | no peak* |
| | 1% benzyl alcohol | |
| B7 | B3 + | 40 |
| | 0.2% m-cresol | |
| C1 | 20 mM sodium phosphate | 51 |
| | 140 mM sodium chloride | |
| | pH 6.2 | |
| C2 | C1 + | 53 |
| | 650 nM polysorbate 80 | |
| C3 | C1 + | 50 |
| | 650 nM polysorbate 20 | |
| C4 | C2 + | 38 |
| | 1% benzyl alcohol | |
| C5 | C2 + | 35 |
| | 0.2% m-cresol | |
| C6 | C3 + | 50 |
| | 1% benzyl alcohol | |
| C7 | C3 + | 43 |
| | 0.2% m-cresol | |
| D1 | 20 mM sodium phosphate | 51 |
| | 140 mM sodium chloride | |
| | pH 7 | |
| D2 | D1 + | 53 |
| | 650 nM polysorbate 80 | |
| D3 | D1 + | 51 |
| | 650 nM polysorbate 20 | |
| D4 | D2 + | 38 |
| | 1% benzyl alcohol | |
| D5 | D2 + | 40 |
| | 0.2% m-cresol | |
| D6 | D3 + | 34 |
| | 1% benzyl alcohol | |
| D7 | D3 + | 40 |
| | 0.2% m-cresol | |

*No peak indicates that no distinct melting point was observed.

Formulations A1, A2, A3, B1, B2, B3, C1, C2, and C3, which do not contain antibacterial agents and have a Tm of 50° C. or above may be more desirable for single dosing. Among the formulations containing antibacterial agents, Formulations A5 and C6, which have a Tm of 50° C. appear to be more desirable for multi-dosing.

### Example 7

### Characterization of EPO Polypeptides

Sialylated glycosylation on a protein may enhance its in vivo pharmacokinetics. Common sialic acids that are expressed as terminal units on all vertebrate glycans typically include N-glycolylneuraminic acid (Neu5Gc) and N-acetylneuraminic acid (Neu5Ac). Sialylation characteristics of basic and/or acidic fractions of EPO polypeptides may be visualized by isoelectric focusing (IEF) or sodium dodecyl sulfate-polyacrylamide gel electrophoresis (SDS-PAGE).

For example, an acidic fraction of EPO polypeptides may be treated with 2 M acetic acid at 80° C. for 3 hours after which the acetic acid is removed under vacuum centrifuge. The treated EPO sample is filtered through a 3K spin filtering unit to remove unhydrolyzed proteins. The flow-through sample is reacted with DMB reagent. The product can be profiled by high-performance liquid chromatography (HPLC) using a C18 column and a fluorescence detector.

For example, sialic acid content of an EPO polypeptide may be determined as follows. Sialic acid is released from EPO polypeptides by mixing with glacial acetic acid. The mixture is incubated at 80° C. for 2 hours. Free sialic acid is labeled with fluorescence dye 1,2-diamino-4,5-methylenoxybenzene (DMB). The florescence labelling is performed by mixing 20 μL of the DMB-thionite solution with 5 μL of the free sialic acid samples. The mixture is incubated at 50° C. for 3 hours. The reaction is stopped by adding 75 μL of distilled, deionized water. The DMB labeled sialic acid is analyzed by HPLC using either a Zorbax SB-C18 column (5μ, 4.6×150 mm) or Extend C18 column (5μ, 4.6×150 mm) (Agilent Technologies), with isocratic mobile phase containing 7% methanol, 9% acetonitrile, and 84% water. All the neuraminic acids, e.g., Neu5Gc (NGNA); Neu5Ac (NANA); Neu5,7Ac2; Neu5,Gc9Ac; Neu5,9Ac2; and Neu5,7(8),9Ac are base line resolved in 30 minutes.

The N-terminal sequence of EPO polypeptides can be confirmed by Edman sequencing.

Isolated EPO polypeptides may be treated with N-Glycanase® (PNGase F) (Catalog No. GKE-5006A, ProZyme, CA) using the manufacturer's instructions to remove N-linked glycans. The deglycosylation process can be monitored by SDS-PAGE until a 19 kD band was visualized, indicating the polypeptide was deglycosylated. The sequences of fragments of the deglycosylated EPO polypeptide may be analyzed using tandem mass spectrometry.

### Example 8

### In Vitro Activity of EPO Polypeptides

The in vitro activity of EPO polypeptides can be analyzed by TF-1 cell proliferation assay. TF-1 cells are factor-dependent human erythroleukemic cells. EPO is one of the factors that promotes TF-1 cell proliferation.

For the proliferation assay, TF-1 cells (ATCC CRL-2003) can be cultivated in RPMI 1640 (Irvine Catalog No. 9160) supplemented with 10% (v/v) Fetal Bovine Serum, 2 mM L-glutamine, 100 units/mL Penicillin, 100 μg/mL Streptomycin, and 2 ng/mL rhGM-CSF (R&D Systems Catalog No. 215-GM). Before treatment with EPO polypeptide, the TF-1 cells are seeded in a 96-flat well plate at 2×10⁵ cells per mL and allowed to attach overnight. The next morning, the cells are treated with different concentrations of acidic and/or basic fractions of EPO polypeptides. Following incubation for 48 hours, MTT reagent (Catalog No. CGD1, Sigma-Aldrich) is added to the cells for another 48-72 hours, according to the manufacturer's instructions. The insoluble purple reaction product is then dissolved with isopropanol, and the plate read at 570 and 690 nm. The proliferation intensity is measured as a difference in optical density between 570 nm and 690 nm (ΔOD) with the background corrected. The concentration of EPO polypeptide that gives half-maximal response (EC50) can be determined for each proliferation curve. The highly acidic fraction of EPO polypeptides may demonstrate lower potency than the basic fraction in the cell-based functional assay due to the shielding effect of glycosylation. Nevertheless, the level of activity may depend on the location of the glycosylation.

### Example 9

### Expression of EPO Receptors

Nucleotide sequences encoding soluble, extracellular domains (ECDs) of feline, canine, or equine EPO receptor polypeptides fused to human Fc can be synthesized, cloned into a mammalian expression vector, and expressed in CHO cells. Supernatant from the cell pellet may analyzed by SD S-PAGE and Western blot using anti-Fc antibody as a probe to confirm expression.

For example, the amino acid sequences of canine and equine EPOR proteins were obtained from the National Center for Biotechnology Information (NCBI) database: SEQ ID NO: 33 (NP 001041576.1) and SEQ ID NO: 37 (XP 023501137.1), respectively. Exemplary ECDs of canine and equine EPOR were identified (SEQ ID NOs: 34, 35, 38, and 39). Canine and equine EPOR ECD polypeptides disclosed herein may be fused to human Fc (e.g., SEQ ID NOs: 36 and 40, respectively).

Exemplary ECDs of feline EPOR are shown as SEQ ID NOs: 42, 43, 45, 46, 48, 49, 51, and 52.

### Example 10

### EPO Polypeptide Binding Assays

EPO polypeptide binding analyses may be performed as follows. Briefly, an EPO receptor ECD fused to human Fc is biotinylated using EZ-Link NHS-LC-biotin (Catalog No. 21336, Thermo Scientific). The free unreacted biotin is removed by dialysis. The biotinylated product is captured on streptavidin sensor tips (Catalog No. 18-509, ForteBio).

The association of different concentrations (e.g., 150, 50, 17, 5.6, and 1.9 nM) of EPO polypeptides may be monitored for a period of time, such as ninety seconds. Dissociation is then monitored for a period of time, such as 600 seconds. A buffer only blank curve is subtracted to correct for any drift. The data are fit to a 1:1 binding model using ForteBio™ data analysis software to determine the kₒₙ (association rate constant), k_{off} (dissociation rate constant) and the K_{d} (dissociation constant).

### Example 11

### EPO Polypeptide Binding to EPOR ELISA Assay

Binding of EPO polypeptides to EPO receptor may be tested by ELISA. For example, a 96-well plate may be coated with a mouse anti-EPO specific antibody (Catalog No. MAB287, clone 9C21D11, R&D Systems) to capture the EPO polypeptides. The EPO-bound wells are incubated with human EPOR-Fc (Catalog No. 963-ER-050, R&D Systems) at a concentration of, for example, 200 ng/mL and the bound EPOR is detected by anti-human Fc HRP conjugated antibody.

As another example, a MaxiSorp 96-well plate may be coated overnight with anti-human EPO antibody (4 μg/mL) at refrigeration temperature (2-8° C.) and blocked with 5% BSA in PBS for 1 hour at room temperature. An EPO polypeptide sample may be prepared in 2-fold serial dilutions starting with a concentration of 500 ng/mL in 1% BSA-PBST (0.05% Tween-20) buffer. The EPO polypeptide dilutions are transferred to each well and incubated at room temperature for 2 hours. An EPO receptor ECD fused to human Fc (e.g., 200 ng/mL in 1% BSA-PBST buffer) is added to each well and binding allowed to proceed for 1 hour at room temperature. A rabbit anti-human Fc antibody and horseradish peroxidase (HRP) conjugate (e.g., 0.2 μg/mL) is used for detection and left in the wells for 1 hour at room temperature. 33,5,5′-Tetramethylbenzidine (TMB) is applied to the wells as the HRP substrate and kept in the well for 5 to 7 minutes for signal development. Binding between EPO polypeptide and the EPO receptor ECD fused to human Fc is determined. The mean detection signal can be plotted against EPO polypeptide concentration and curve fit analysis performed.

### Example 12

### Administration of EPO Polypeptides to Companion Animals

A single dose of any of the EPO polypeptides described herein may be assessed in normal or anemic companion animals, e.g., cats, dogs, and/or horses, after subcutaneous administration of 1 μg/kg, 3 μg/kg, 10 μg/kg, or greater than 10 μg/kg compared to a control. The dose escalation may be used to determine or compare pharmacokinetic, pharmacodynamic, safety and/or efficacy profiles. Absolute reticulocyte percentages may be measured as an indicator of EPO bioactivity. In brief, EPO binds to EPO receptor on erythroid cells and the dimerization of the receptor activates the JAK2 pathway and signaling of erythropoiesis. Erythroid cells differentiate into reticulocytes, then red blood cells. Thus, an increase in EPO bioactivity and erythropoiesis is evidenced by an increase in the percentage of absolute reticulocytes.

### Example 13

### Efficacy Study of EPO Polypeptides in Anemic Companion Animals

An open-label, historical controlled (compared to companion animals' post-treatment and pre-treatment data), pilot efficacy study may be conducted to evaluate the effectiveness of any of the EPO polypeptides described herein on red blood cells (RBC), reticulocytes, and Quality of Life (QoL) in client-owned companion animals with International Renal Interest Society (IRIS) Stage 3 Chronic Kidney Disease (CKD) and anemia. Safety may also be evaluated by the collation of any adverse events (AE) and the presence of neutralizing antibodies.

Inclusion Criteria may include the following:

The companion animal:
1. Is manageable and cooperative with study procedures
2. Has rapidly progressive CKD with a 25% increase in fasting serum creatinine between two consecutive evaluations
3. Is at least 1 year of age on Day 0 and is: any gender; intact or neutered; non-pregnant, non-lactating; any breed and any weight
4. Has IRIS Stage 3 CKD, defined as:
   a) A Screening Visit fasting serum creatinine of 2.9-5.0 mg/dL and a previous medical history of serum creatinine of 2.9-5.0 mg/dL within 6 months of Day 0 (fasting or unfasted); and
   b) Urine specific gravity (USG)<1.035
5. Has non-regenerative anemia and a 15-30% HCT
6. Is receiving standard of care therapy for CKD

Exclusion Criteria may include the following:

The companion animal:
1. Resides mostly outdoors (>60% of each day is spent outside)
2. Has rapidly progressive CKD with a 25% increase in fasting serum creatinine between two consecutive evaluations
3. Has ever been treated with an erythropoietin stimulating agent
4. Has been administered whole or packed red blood cells within 6 weeks of Day 0;
5. Has a urinary tract infection (UTI) with the following exception: companion animals with a UTI may be enrolled post-treatment with the appropriate antibiotic therapy (based on culture/sensitivity) for 3 weeks and repeat negative culture
6. Has any of the following diseases/conditions:
   Neoplasia
   Liver disease
   Feline leukemia virus (FeLV)
   Feline immunodeficiency virus (FIV)
   Diabetes mellitus (DM)
   Hyperthyroidism
   Hematocrit <15%
   Systemic blood pressure >160 mmHg
7. Requires a new prescription or a prescription change (dose or dose frequency) to an existing concurrent medication or therapy for CKD two weeks before Day 0.

Companion animals may be administered a EPO polypeptide subcutaneously twice at a starting dose approximately 7-10 days apart, and followed for six weeks. Companion animals may be concurrently administrated iron dextran.

The following data may be collected and/or evaluated at all visits (scheduled or unscheduled): physical examination with a medical history, quality of life (vitality, comfort, and emotional wellbeing), appetite, activity (Vetrax activity sensor affixed to a neck collar), blood pressure, and owner diary of observed events. At initial Screening and Week 6 Visits, hematology, biochemistry, urinalysis with urine protein to creatinine ratio, and SDMA assessments may be made. Urine culture ±sensitivity may be assessed at baseline and as needed throughout the study. Hematocrit may be assessed in-house at all scheduled and unscheduled visits.

The change in baseline hematocrit, body weight, SDMA, serum creatinine renal biomarker, or any other measure may be determined.

### Example 14

### Variant Canine IgG Fc Polypeptides for Increased Protein A Binding and/or Decreased Complement Binding and/or Decreased CD16 Binding

Purification of antibodies using Protein A affinity is a well-developed process. However, among four subtypes of canine IgG, only IgG-B Fc (e.g., SEQ ID NO: 54 or SEQ ID NO: 55) has Protein A binding affinity. Canine IgG-A Fc (e.g., SEQ ID NO: 53), IgG-C Fc (e.g., SEQ ID NO: 56 or SEQ ID NO: 57), and IgG-D Fc (e.g., SEQ ID NO: 58) have weak or no measurable Protein A binding affinity. Variant canine IgG-A Fc, IgG-C Fc, and IgG-D Fc polypeptides were designed for altered Protein A binding.

In addition, canine IgG-B Fc and IgG-C Fc have complement activity and bind to C1q, while canine IgG-A Fc and IgG-D Fc have weak or no measurable binding affinity to C1q. To potentially reduce the C1q binding and/or potentially reduce complement-mediated immune responses, variant canine IgG-B Fc and IgG-C Fc polypeptides were designed.

Furthermore, canine IgG-B Fc and IgG-C Fc have CD16 binding activity. To potentially reduce the binding of CD16 to IgG-B Fc and IgG-C Fc, and/or potentially reduce ADCC, variant canine IgG-B Fc and IgG-C Fc polypeptides were designed.

Table 11, below summarizes the Protein A and C1q binding characteristics of canine IgG Fc subtypes. Notably, none of the wild-type canine IgG Fc subtypes lacks C1q binding and binds Protein A.

TABLE 11

| Wild-type | Protein A | C1q | CD16 |
|---|---|---|---|
| Canine IgGFc | Binding | Binding | Binding |
| IgG-A Fc | − | − | − |
| IgG-B Fc | + | + | + |
| IgG-C Fc | − | + | + |
| IgG-D Fc | − | − | − |

(−) denotes low or no measurable binding activity.

Using three-dimensional protein modeling and protein sequence analysis, the sequences of canine IgG-B Fc that are likely in contact with Protein A were identified. Two approaches were used to design variant canine IgG-A, IgG-C, and IgG-D Fc polypeptides for increased Protein A binding. For the first approach, variant canine IgG-A, IgG-C, and IgG-D Fc polypeptides were designed to have the same Protein A binding motif sequences as canine IgG-B Fc (e.g., SEQ ID NO: 59, SEQ ID NO: 60 and SEQ ID NO: 61, respectively). For the second approach, variant canine IgG-A Fc I(21)T/Q(207)H (SEQ ID NO: 62), variant canine IgG-C Fc I(21)T (SEQ ID NO: 63), and variant canine IgG-D Fc I(21)T/Q(207)H (SEQ ID NO: 64) were designed with one or two amino acid substitutions in the Protein A binding region to correspond with the canine IgG-B Fc sequence.

In addition, variant canine IgG-A Fc, IgG-C Fc, and IgG-D Fc polypeptides with increased Protein A binding may be prepared having one or more of the amino acid substitutions listed in Table 12.

TABLE 12
Variant Canine IgG Fc Amino Acid Substitutions* (Protein A+)

| Canine IgG-A Fc | Canine IgG-C Fc | Canine IgG-D Fc |
|---|---|---|
| (SEQ ID NO: 53) | (SEQ ID NO: 56) | (SEQ ID NO: 58) |
| Ile (21) Thr | Ile (21) Thr | Ile (21) Thr |
| Arg (23) Leu | Val (23) Leu | Arg (23) Leu |
| Thr (25) Ala | Thr (24) Ile | Thr (25) Ala |
| Glu (80) Gly | | Glu (80) Gly |
| Thr (205) Ala | | Gln (207) His |
| Gln (207) His | | |

*The amino acid positions listed are relative to the SEQ ID NO. indicated.

To potentially reduce the binding of C1q to canine IgG-B Fc and IgG-C Fc, and/or potentially reduce complement-mediated immune responses, variant canine IgG-B Fc and IgG-C Fc polypeptides may be prepared having an amino acid substitution of Lys with any amino acid except Lys at an amino acid position corresponding to position 93 of SEQ ID NO: 54 or of SEQ ID NO: 56, respectively. These amino acid substitutions were identified after analysis of the protein sequence and 3-D structure modeling of canine IgG-B Fc and IgG-C Fc compared to canine IgG-A Fc and IgG-D Fc, which are understood to not exhibit complement activity. For example, variant canine IgG-B Fc K(93)R (SEQ ID NO: 65) and variant canine IgG-C Fc K(93)R (SEQ ID NO: 66) may be prepared. Reduced binding between human C1q and a fusion protein comprising variant canine IgG-B Fc K(93)R was observed when compared to a fusion protein comprising wild-type canine IgG-B Fc.

To potentially reduce the binding of CD16 to IgG-B Fc and IgG-C Fc, and/or potentially reduce ADCC, variant canine IgG-B Fc and IgG-C Fc polypeptides may be prepared having one or more of the amino acid substitutions listed in Table 13 (e.g., SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and/or SEQ ID NO: 81). The amino acid substitution(s) were identified after analysis of the protein sequence and 3-D structure modeling of canine IgG-B and IgG-C compared to IgG-A and IgG-D, which are understood to not exhibit ADCC activity.

TABLE 13

Original residue position*

| Canine IgG-B Fc | Canine IgG-C Fc | |
|---|---|---|
| (SEQ ID NO: 54) | (SEQ ID NO: 56) | Substitution(s) |
| Met (5) | Leu (5) | Any amino acid |
| | | except original |
| | | residue, such as Pro |
| Asp (38) | Asp (38) | Any amino acid |
| | | except original |
| | | residue, such as |
| | | Gly |
| Pro (39) | Pro (39) | Any amino acid |
| | | except original |
| | | residue, such as |
| | | Arg |
| Lys (97) | Lys (97) | Any amino acid |
| | | except original |
| | | residue, such as Ile |
| Ala (98) | Ala (98) | Any amino acid |
| | | except original |
| | | residue, such as |
| | | Gly |

*The amino acid positions listed are relative to the SEQ ID NO. indicated.

Since wild-type canine IgG-C Fc lacks Protein A binding and has C1q binding, a double variant canine IgG-C Fc that binds Protein A and has reduced binding to C1q may be prepared by combining one or more of the amino acid substitutions listed in Table 12 with a K(93)R substitution or K(93)X substitution, wherein X is any amino acid except Lys (e.g., SEQ ID NO: 82). A double variant canine IgG-B Fc or double variant canine IgG-C Fc with reduced binding to C1q and reduced binding to CD16 may be prepared by combining one or more of the amino acid substitutions listed in Table 13 with a K(93)R substitution or K(93)X substitution, wherein X is any amino acid except Lys (e.g., SEQ ID NO: 83, SEQ ID NO: 84, SEQ ID NO: 85, and/or SEQ ID NO: 86). A triple variant canine-IgG-C Fc that binds Protein A and has reduced binding to C1q and CD16 may be prepared by combining one or more of the amino acid substitutions listed in Table 12 and one or more of the amino acid substitutions listed in Table 13 with a K(93)R substitution or K(93)X substitution, wherein X is any amino acid except Lys.

The binding of any variant canine IgG Fc to Protein A, CD16, and/or C1q may be determined and compared to the binding of another IgG Fc to Protein A, CD16, and/or C1q (e.g., the corresponding wild-type canine IgG Fc, another wild-type or variant canine IgG Fc, or a wild-type or variant IgG Fc of another companion animal, etc.).

Binding analysis may be performed using an Octet biosensor. Briefly, the target molecule (e.g., Protein A, C1q, CD16, etc.) may be biotinylated and free unreacted biotin removed (e.g., by dialysis). The biotinylated target molecule is captured on streptavidin sensor tips. Association of the target molecule with various concentrations (e.g., 10 μg/mL) of IgG Fc polypeptide is monitored for a specified time or until steady state is reached. Dissociation is monitored for a specified time or until steady state is reached. A buffer only blank curve may be subtracted to correct for any drift. The data are fit to a 1:1 binding model using ForteBio′ data analysis software to determine the kₒₙ, k_{off}, and the K_{d}.

### Example 15

### Variant Equine IgG Fc Polypeptides for Increased Protein a Binding and/or Decreased Complement Binding

Of the seven subtypes of equine IgG, IgG1 Fc (e.g., SEQ ID NO: 87), IgG3 Fc (e.g., SEQ ID NO: 90), IgG4 Fc (e.g., SEQ ID NO: 91), IgG7 Fc (e.g., SEQ ID NO: 94) have Protein A binding affinity. Equine IgG2 Fc (e.g., SEQ ID NO: 88, SEQ ID NO: 89), IgG5 Fc (e.g., SEQ ID NO: 92), and IgG6 Fc (e.g., SEQ ID NO: 93) have weak or no measurable Protein A binding affinity. Variant equine IgG2 Fc, IgG5 Fc, and IgG6 Fc polypeptides were designed for altered Protein A binding.

In addition, equine IgG2 Fc, IgG5 Fc, and IgG6 Fc have weak or no measurable binding affinity to C1q, while equine IgG1 Fc, IgG3 Fc, IgG4 Fc, and IgG7 Fc bind to C1q. To potentially reduce the C1q binding and/or potentially reduce complement-mediated immune responses, variant equine IgG1 Fc, IgG3 Fc, IgG4 Fc, and IgG7 Fc polypeptides were designed.

Table 14, below summarizes the Protein A and C1q binding characteristics of equine IgG Fc subtypes. Notably, none of the wild-type equine IgG Fc subtypes lacks C1q binding and binds Protein A.

TABLE 14

| Wild-type | Protein A | C1q |
|---|---|---|
| Equine IgG Fc | Binding | Binding |
| IgG1 Fc | + | + |
| IgG2 Fc | − | − |
| IgG3 Fc | + | + |
| IgG4 Fc | + | + |
| IgG5 Fc | − | − |
| IgG6 Fc | − | − |
| IgG7 Fc | + | + |

(−) denotes low or no measurable binding activity.

Using three-dimensional protein modeling and protein sequence analysis, the sequences of equine IgG1 Fc, IgG3 Fc, IgG4 Fc, and IgG7 Fc that are likely in contact with Protein A were identified. Variant equine IgG2 Fc, IgG5 Fc, and IgG6 Fc polypeptides with increased Protein A binding may be prepared having one or more of the amino acid substitutions listed in Table 15.

TABLE 15
Variant Equine IgG Fc Amino Acid Substitutions* (Protein A+)

| Equine IgG2 Fc | Equine IgG5 Fc | Equine Ig6 Fc |
|---|---|---|
| (SEQ ID NO: 88) | (SEQ ID NO: 92) | (SEQ ID NO: 93) |
| Ala (15) Thr | Val (199) Leu | Ile (199) Leu |
| Phe (203) Tyr | Glu (200) Tyr | Arg (200) His |
| | | His (201) Asn |
| | | Thr (202) His |

*The amino acid positions listed are relative to the SEQ ID NO. indicated

For example, variant equine IgG2 Fc, IgG5 Fc, and IgG6 Fc polypeptides were designed with one or multiple amino acid substitutions in the Protein A binding region to correspond with the sequence of wild-type equine IgG Fc, which does bind Protein A. Variant equine IgG2 Fc F(203)Y (SEQ ID NO: 95); variant equine IgG2 Fc A(15)T/F(203)Y (SEQ ID NO: 96); variant equine IgG5 Fc V(199)L/E(200)Y (SEQ ID NO: 97); and variant equine IgG6 Fc I(199)L/R(200)H/H(201)N/T(202)H (SEQ ID NO: 98) with increased Protein A binding may be prepared.

To potentially reduce the binding of C1q to equine IgG1 Fc, IgG3 Fc, IgG4 Fc, and IgG7 Fc, and/or potentially reduce complement-mediated immune responses, variant canine IgG1 Fc, IgG3 Fc, IgG4 Fc, and IgG7 Fc polypeptides may be prepared having an amino acid substitution of Lys with any amino acid except Lys at an amino acid position corresponding to position 87 of SEQ ID NO: 97, of SEQ ID NO: 90, of SEQ ID NO: 91, of SEQ ID NO: 94, respectively. These amino acid substitutions were identified after analysis of the protein sequence and 3-D structure modeling of equine IgG1 Fc, IgG3 Fc, IgG4 Fc, and IgG7 Fc compared to equine IgG2 Fc, IgG5 Fc, and IgG6 Fc, which are understood to not exhibit complement activity. For example, variant equine IgG1 Fc K(87)S (SEQ ID NO: 99), variant equine IgG3 Fc K(87)S (SEQ ID NO: 100), variant equine IgG4 Fc K(87)S (SEQ ID NO: 101), and variant equine IgG7 Fc K(87)S (SEQ ID NO: 102) may be prepared.

The binding of any variant equine IgG Fc to Protein A and/or C1q may be determined and compared to the binding of another IgG Fc to Protein A and/or C1q (e.g., the corresponding wild-type equine IgG Fc, another wild-type or variant equine IgG Fc, or a wild-type or variant IgG Fc of another companion animal, etc.). The binding assay described in Example 14 may be used.

### Example 16

### Variant Feline IgG Fc Polypeptides for Decreased Complement Binding

Each of the three subtypes of feline IgG, IgG1a Fc (SEQ ID NO: 103 or SEQ ID NO: 104), IgG1b Fc (SEQ ID NO: 105 or SEQ ID NO: 106), and IgG2 Fc (SEQ ID NO: 107) have Protein A binding affinity. However, only feline IgG2 Fc has weak or no measurable binding affinity to C1q, while feline IgG1a Fc, IgG1b Fc bind to C1q. To potentially reduce the C1q binding and/or potentially reduce complement-mediated immune responses, variant feline IgG1a Fc and IgG1b Fc polypeptides were designed.

Table 16, below summarizes the Protein A and C1q binding characteristics of feline IgG Fc subtypes. Notably, none of the wild-type equine IgG Fc subtypes lacks C1q binding and binds Protein A.

TABLE 16

| Wild-type | Protein A | C1q |
|---|---|---|
| Feline IgG Fc | Binding | Binding |
| IgG1a Fc | + | + |
| IgG1b Fc | + | + |
| IgG2 Fc | + | − |

(−) denotes low or no measurable binding activity.

To potentially reduce the binding of C1q to feline IgG1a Fc and IgG1b Fc, and/or potentially reduce complement-mediated immune responses, variant feline IgG1a Fc and IgG1b Fc polypeptides may be prepared having an amino acid substitution of Pro with any amino acid except Pro at an amino acid position corresponding to position 198 of SEQ ID NO: 103, of SEQ ID NO: 104, of SEQ ID NO: 105, or of SEQ ID NO: 106. These amino acid substitutions were identified after analysis of the protein sequence and 3-D structure modeling of feline IgG1a Fc and IgG1b Fc compared to feline IgG2 Fc, which is understood to not exhibit complement activity. For example, variant feline IgG1a Fc P(198)A (e.g., SEQ ID NO: 108 or SEQ ID NO: 109) and variant feline IgG1b Fc P(198)A (e.g., SEQ ID NO: 110 or SEQ ID NO: 111) may be prepared.

The binding of any variant feline IgG Fc to C1q may be determined and compared to the binding of another IgG Fc to C1q (e.g., the corresponding wild-type feline IgG Fc, another wild-type or variant feline IgG Fc, or a wild-type or variant IgG Fc of another companion animal, etc.). The binding assay described in Example 14 may be used.

### Example 17

### N-Linked Glycosylation Sites for Feline EPO E44

Wild-type feline EPO E44 precursor form (SEQ ID NO: 7 or “wild-type feline EPO E44”) has three N-linked glycosylation sites at amino acid positions 50-52, 64-66, and 109-111, which correspond to amino acid positions 24-26, 38-40, and 83-85 of wild-type feline EPO E44 mature form (SEQ ID NO: 8 or “wild-type feline EPO E18”).

Additional N-linked glycosylation sites may be also introduced into wild-type feline EPO E44 and wild-type feline EPO E18 amino acid sequences. For example, one, two, three, four, five, or six additional N-linked glycosylation sites may be introduced into wild-type feline EPO E44/E18 amino acid sequences. The N-linked glycosylation site may have a consensus sequence of Asn-Xaa-Ser/Thr, where Xaa is any amino acid except proline. Addition of one or more glycosylation sites may increase the molecular size of a feline EPO molecule, provide more sialylation sites, and/or improve the half-life of the molecule in an animal's serum.

Table 17 lists amino acid substitutions of wild-type feline EPO E44 and E18 that may be used to generate one or more additional N-linked glycosylation sites. Exemplary amino acid sequences of feline EPO polypeptides having at least one additional N-linked glycosylation site include SEQ ID NOs: 112-119.

TABLE 17

| | Amino acid substitutions for N-linked glycosylation sites | |
|---|---|---|
| | Based on wt feline | Based on wt feline |
| | EPO E44 precursor sequence | EPO E18 mature sequence |
| Analog No. | (SEQ ID NO: 7) | (SEQ ID NO: 8) |
| 1 | N47S49 | N21S23 |
| 2 | N47T49 | N21T23 |
| 3 | N55S57 | N29S31 |
| 4 | N55T57 | N29T31 |
| 5 | N56S58 | N30S32 |
| 6 | N56T58 | N30T32 |
| 7 | N60 | N34 |
| 8 | N60T62 | N34T36 |
| 9 | N61S63 | N35S37 |
| 10 | N61T63 | N35T37 |
| 11 | N79S81 | N53S55 |
| 12 | N79T81 | N53T55 |
| 13 | N81S83 | N55S57 |
| 14 | N81T83 | N55T57 |
| 15 | N82S84 | N56S58 |
| 16 | N82T84 | N56T58 |
| 17 | N91S93 | N65S67 |
| 18 | N91T93 | N65T67 |
| 19 | N92S94 | N66S68 |
| 20 | N92T94 | N66T68 |
| 21 | N97S99 | N71S73 |
| 22 | N97T99 | N71T73 |
| 23 | N98S100 | N72S74 |
| 24 | N98T100 | N72T74 |
| 25 | N99S101 | N73S75 |
| 26 | N99T101 | N73T75 |
| 27 | N112*X113 | N86*X87 |
| 28 | N112*X113T114 | N86*X87T88 |
| 29 | N113S115 | N87S89 |
| 30 | N113T115 | N87T89 |
| 31 | *X113N114S116 | *X87N88S90 |
| 32 | *X113N114 | *X87N88 |
| 33 | N115S117 | N89S91 |
| 34 | N115T117 | N89T91 |
| 35 | N116S118 | N90S92 |
| 36 | N116T118 | N90T92 |
| 37 | N137S139 | N111S113 |
| 38 | N137T139 | N111T113 |
| 39 | N138S140 | N112S114 |
| 40 | N138T140 | N112T114 |
| 41 | N140S142 | N114S116 |
| 42 | N140T142 | N114T116 |
| 43 | N141S143 | N115S117 |
| 44 | N141T143 | N115T117 |
| 45 | N142S144 | N116S118 |
| 46 | N142T144 | N116T118 |
| 47 | N143S145 | N117S119 |
| 48 | N143 | N117 |
| 49 | N144 | N118 |
| 50 | N144T146 | N118T120 |
| 51 | N145S147 | N119S121 |
| 52 | N145T147 | N119T121 |
| 53 | N146S148 | N120S122 |
| 54 | N146T148 | N120T122 |
| 55 | N147*X148S149 | N121*X122S123 |
| 56 | N147*X148T149 | N121*X122T123 |
| 57 | N148S150 | N122S124 |
| 58 | N148T150 | N122T124 |
| 59 | N149S151 | N123S125 |
| 71 | *X148N149 | *X122N123 |
| 72 | *X148N149S151 | *X122N123S125 |
| 60 | N149 | N123 |
| 61 | N150 | N124 |
| 62 | N150T152 | N124T126 |
| 63 | N161S163 | N135S137 |
| 64 | N161 | N135 |
| 65 | N162S164 | N136S138 |
| 66 | N162T164 | N136T138 |
| 67 | N184S186 | N158S160 |
| 68 | N184T186 | N158T160 |
| 69 | N186S188 | N162S164 |
| 70 | N186T188 | N162T164 |

*X indicates any amino acid except proline (such as E, V, S, A, etc.)

### Example 18

### Identification and Removal of Unpaired Cysteine of Feline EPO

An unpaired cysteine may cause undesirable effects, such as disulfide scrambling (incorrect disulfide bond formation) and intermolecular covalent disulfide binding. Wild-type feline EPO was determined to have two cysteine pairs and one unpaired cysteine at position 139 of the mature feline EPO sequence (SEQ ID NO: 8), which corresponds to position 165 of the precursor feline EPO sequence (SEQ ID NO: 7). The cysteine at position 139 of the mature sequence may be replaced with any other amino acid, such as threonine, serine, or alanine (see e.g., SEQ ID NOs: 122 and 123).

1. An erythropoietin (EPO) polypeptide comprising the amino acid sequence of SEQ ID NO: 1, SEQ ID NO: 2, SEQ ID NO: 3, SEQ ID NO: 4, SEQ ID NO: 7, or SEQ ID NO: 8, except for the presence of at least one N-linked glycosylation site not present in SEQ ID NO: 1, SEQ ID NO: 2, SEQ ID NO: 3, SEQ ID NO: 4, SEQ ID NO: 7, or SEQ ID NO: 8, wherein the N-linked glycosylation site comprises the sequence asparagine-xaa-serine or asparagine-xaa-threonine, wherein xaa is any amino acid except proline, and wherein one N-linked glycosylation site does not overlap with another N-linked glycosylation site.

2. The EPO polypeptide of claim 1, wherein each of the at least one N-linked glycosylation sites is present at:
a) a position selected from position 47-49, 55-57, 56-58, 60-62, 61-63, 79-81, 81-83, 82-84, 91-93, 92-94, 97-99, 98-100, 99-101, 112-114, 113-115, 114-116, 115-117, 116-118, 137-139, 138-140, 140-142, 141-143, 142-144, 143-145, 144-146, 145-147, 146-148, 147-149, 148-150, 149-151, 150-152, 161-163, 162-164, 184-186, and 186-188 of SEQ ID NO: 1, SEQ ID NO: 3, or SEQ ID NO: 7; or
b) a position selected from position 21-23, 29-31, 30-32, 34-36, 35-37, 53-55, 55-57, 56-58, 65-67, 66-68, 71-73, 72-74, 73-75, 86-88, 87-89, 88-90, 89-91, 90-92, 111-113, 112-114, 114-116, 115-117, 116-118, 117-119, 118-120, 119-121, 120-122, 121-123, 122-124, 123-125, 124-126, 135-137, 136-138, 158-160, and 162-164 of SEQ ID NO: 2, or SEQ ID NO: 4, or SEQ ID NO: 8.

3. The EPO polypeptide of claim 1 or claim 2 comprising an amino acid except proline at a position corresponding to position 113 or position 148 of SEQ ID NO: 1, SEQ ID NO: 3, or SEQ ID NO: 7, or at a position corresponding to position 87 or position 122 of SEQ ID NO: 2, SEQ ID NO: 4, or SEQ ID NO: 8.

4. The EPO polypeptide of any one of claims 1 to 3 comprising valine or glutamic acid at a position corresponding to position 113 or position 148 of SEQ ID NO: 1, SEQ ID NO: 3, or SEQ ID NO: 7, or at a position corresponding to position 87 or position 122 of SEQ ID NO: 2, SEQ ID NO: 4, or SEQ ID NO: 8.

5. The EPO polypeptide of any one of claims 1 to 4 comprising:
a) asparagine at a position corresponding to position 47 of SEQ ID NO: 1, SEQ ID NO: 3, or SEQ ID NO: 7, any amino acid except proline at a position corresponding to position 48 of SEQ ID NO: 1, SEQ ID NO: 3, or SEQ ID NO: 7, and serine or threonine at a position corresponding to position 49 of SEQ ID NO: 1, SEQ ID NO: 3, or SEQ ID NO: 7; or
b) asparagine at a position corresponding to position 21 of SEQ ID NO: 2, SEQ ID NO: 4, or SEQ ID NO: 8, any amino acid except proline at a position corresponding to position 22 of SEQ ID NO: 2, SEQ ID NO: 4, or SEQ ID NO: 8, and serine or threonine at a position corresponding to position 23 of SEQ ID NO: 2, SEQ ID NO: 4, or SEQ ID NO: 8.

6. The EPO polypeptide of any one of claims 1 to 5 comprising:
a) asparagine at a position corresponding to position 55 of SEQ ID NO: 1, SEQ ID NO: 3, or SEQ ID NO: 7, any amino acid except proline at a position corresponding to position 56 of SEQ ID NO: 1, SEQ ID NO: 3, or SEQ ID NO: 7, and serine or threonine at a position corresponding to position 57 of SEQ ID NO: 1, SEQ ID NO: 3, or SEQ ID NO: 7; or
b) asparagine at a position corresponding to position 29 of SEQ ID NO: 2, SEQ ID NO: 4, or SEQ ID NO: 8, any amino acid except proline at a position corresponding to position 30 of SEQ ID NO: 2, SEQ ID NO: 4, or SEQ ID NO: 8, and serine or threonine at a position corresponding to position 31 of SEQ ID NO: 2, SEQ ID NO: 4, or SEQ ID NO: 8.

7. The EPO polypeptide of any one of claims 1 to 6 comprising:
a) asparagine at a position corresponding to position 56 of SEQ ID NO: 1, SEQ ID NO: 3, or SEQ ID NO: 7, any amino acid except proline at a position corresponding to position 57 of SEQ ID NO: 1, SEQ ID NO: 3, or SEQ ID NO: 7, and serine or threonine at a position corresponding to position 58 of SEQ ID NO: 1, SEQ ID NO: 3, or SEQ ID NO: 7; or
b) asparagine at a position corresponding to position 30 of SEQ ID NO: 2, SEQ ID NO: 4, or SEQ ID NO: 8, any amino acid except proline at a position corresponding to position 31 of SEQ ID NO: 2, SEQ ID NO: 4, or SEQ ID NO: 8, and serine or threonine at a position corresponding to position 32 of SEQ ID NO: 2, SEQ ID NO: 4, or SEQ ID NO: 8.

8. The EPO polypeptide of any one of claims 1 to 7 comprising:
a) asparagine at a position corresponding to position 60 of SEQ ID NO: 1, SEQ ID NO: 3, or SEQ ID NO: 7, any amino acid except proline at a position corresponding to position 61 of SEQ ID NO: 1, SEQ ID NO: 3, or SEQ ID NO: 7, and serine or threonine at a position corresponding to position 62 of SEQ ID NO: 1, SEQ ID NO: 3, or SEQ ID NO: 7; or
b) asparagine at a position corresponding to position 34 of SEQ ID NO: 2, SEQ ID NO: 4, or SEQ ID NO: 8, any amino acid except proline at a position corresponding to position 35 of SEQ ID NO: 2, SEQ ID NO: 4, or SEQ ID NO: 8, and serine or threonine at a position corresponding to position 36 of SEQ ID NO: 2, SEQ ID NO: 4, or SEQ ID NO: 8.

9. The EPO polypeptide of any one of claims 1 to 8 comprising:
a) asparagine at a position corresponding to position 61 of SEQ ID NO: 1, SEQ ID NO: 3, or SEQ ID NO: 7, any amino acid except proline at a position corresponding to position 62 of SEQ ID NO: 1, SEQ ID NO: 3, or SEQ ID NO: 7, and serine or threonine at a position corresponding to position 63 of SEQ ID NO: 1, or SEQ ID NO: 3, or SEQ ID NO: 7; or
b) asparagine at a position corresponding to position 35 of SEQ ID NO: 2, SEQ ID NO: 4, or SEQ ID NO: 8, any amino acid except proline at a position corresponding to position 36 of SEQ ID NO: 2, SEQ ID NO: 4 or SEQ ID NO: 8, and serine or threonine at a position corresponding to position 37 of SEQ ID NO: 2, SEQ ID NO: 4, or SEQ ID NO: 8.

10. The EPO polypeptide of any one of claims 1 to 9 comprising:
a) asparagine at a position corresponding to position 79 of SEQ ID NO: 1, SEQ ID NO: 3, or SEQ ID NO: 7, any amino acid except proline at a position corresponding to position 80 of SEQ ID NO: 1, SEQ ID NO: 3, or SEQ ID NO: 7, and serine or threonine at a position corresponding to position 81 of SEQ ID NO: 1, SEQ ID NO: 3, or SEQ ID NO: 7; or
b) asparagine at a position corresponding to position 53 of SEQ ID NO: 2, SEQ ID NO: 4, or SEQ ID NO: 8, any amino acid except proline at a position corresponding to position 54 of SEQ ID NO: 2, SEQ ID NO: 4, or SEQ ID NO: 8, and serine or threonine at a position corresponding to position 55 of SEQ ID NO: 2, SEQ ID NO: 4, or SEQ ID NO: 8.

11. The EPO polypeptide of any one of claims 1 to 10 comprising:
a) asparagine at a position corresponding to position 81 of SEQ ID NO: 1, SEQ ID NO: 3 or SEQ ID NO: 7, any amino acid except proline at a position corresponding to position 82 of SEQ ID NO: 1, SEQ ID NO: 3, or SEQ ID NO: 7, and serine or threonine at a position corresponding to position 83 of SEQ ID NO: 1, SEQ ID NO: 3, or SEQ ID NO: 7; or
b) asparagine at a position corresponding to position 55 of SEQ ID NO: 2, SEQ ID NO: 4, or SEQ ID NO: 8, any amino acid except proline at a position corresponding to position 56 of SEQ ID NO: 2, SEQ ID NO: 4, or SEQ ID NO: 8, and serine or threonine at a position corresponding to position 57 of SEQ ID NO: 2, SEQ ID NO: 4, or SEQ ID NO: 8.

12. The EPO polypeptide of any one of claims 1 to 11 comprising:
a) asparagine at a position corresponding to position 82 of SEQ ID NO: 1, SEQ ID NO: 3, or SEQ ID NO: 7, any amino acid except proline at a position corresponding to position 83 of SEQ ID NO: 1, SEQ ID NO: 3, or SEQ ID NO: 7, and serine or threonine at a position corresponding to position 84 of SEQ ID NO: 1, SEQ ID NO: 3, or SEQ ID NO: 7; or
b) asparagine at a position corresponding to position 56 of SEQ ID NO: 2, SEQ ID NO: 4, or SEQ ID NO: 8, any amino acid except proline at a position corresponding to position 57 of SEQ ID NO: 2, SEQ ID NO: 4, or SEQ ID NO: 8, and serine or threonine at a position corresponding to position 58 of SEQ ID NO: 2, SEQ ID NO: 4, or SEQ ID NO: 8.

13. The EPO polypeptide of any one of claims 1 to 12 comprising:
a) asparagine at a position corresponding to position 91 of SEQ ID NO: 1, SEQ ID NO: 3, or SEQ ID NO: 7, any amino acid except proline at a position corresponding to position 92 of SEQ ID NO: 1, SEQ ID NO: 3, or SEQ ID NO: 7, and serine or threonine at a position corresponding to position 93 of SEQ ID NO: 1, SEQ ID NO: 3, or SEQ ID NO: 8; or
b) asparagine at a position corresponding to position 65 of SEQ ID NO: 2, SEQ ID NO: 4, or SEQ ID NO: 8, any amino acid except proline at a position corresponding to position 66 of SEQ ID NO: 2, SEQ ID NO: 4, or SEQ ID NO: 8, and serine or threonine at a position corresponding to position 67 of SEQ ID NO: 2, SEQ ID NO: 4, or SEQ ID NO: 8.

14. The EPO polypeptide of any one of claims 1 to 13 comprising:
a) asparagine at a position corresponding to position 92 of SEQ ID NO: 1, SEQ ID NO: 3, or SEQ ID NO: 7, any amino acid except proline at a position corresponding to position 93 of SEQ ID NO: 1, SEQ ID NO: 3, or SEQ ID NO: 7, and serine or threonine at a position corresponding to position 94 of SEQ ID NO: 1, SEQ ID NO: 3, or SEQ ID NO: 7; or
b) asparagine at a position corresponding to position 66 of SEQ ID NO: 2, SEQ ID NO: 4, or SEQ ID NO: 8, any amino acid except proline at a position corresponding to position 67 of SEQ ID NO: 2, SEQ ID NO: 4, or SEQ ID NO: 8, and serine or threonine at a position corresponding to position 68 of SEQ ID NO: 2, SEQ ID NO: 4, or SEQ ID NO: 8.

15. The EPO polypeptide of any one of claims 1 to 14 comprising:
a) asparagine at a position corresponding to position 97 of SEQ ID NO: 1, SEQ ID NO: 3, or SEQ ID NO: 7, any amino acid except proline at a position corresponding to position 98 of SEQ ID NO: 1, SEQ ID NO: 3, or SEQ ID NO: 7, and serine or threonine at a position corresponding to position 99 of SEQ ID NO: 1, SEQ ID NO: 3, or SEQ ID NO: 7; or
b) asparagine at a position corresponding to position 71 of SEQ ID NO: 2, SEQ ID NO: 4, or SEQ ID NO: 8, any amino acid except proline at a position corresponding to position 92 of SEQ ID NO: 2, SEQ ID NO: 4, or SEQ ID NO: 8, and serine or threonine at a position corresponding to position 73 of SEQ ID NO: 2, SEQ ID NO: 4, or SEQ ID NO: 8.

16. The EPO polypeptide of any one of claims 1 to 15 comprising:
a) asparagine at a position corresponding to position 98 of SEQ ID NO: 1, SEQ ID NO: 3, or SEQ ID NO: 7, any amino acid except proline at a position corresponding to position 99 of SEQ ID NO: 1, SEQ ID NO: 3, or SEQ ID NO: 7, and serine or threonine at a position corresponding to position 100 of SEQ ID NO: 1, SEQ ID NO: 3, or SEQ ID NO: 7; or
b) asparagine at a position corresponding to position 72 of SEQ ID NO: 2, SEQ ID NO: 4, or SEQ ID NO: 8, any amino acid except proline at a position corresponding to position 73 of SEQ ID NO: 2, SEQ ID NO: 4, or SEQ ID NO: 8, and serine or threonine at a position corresponding to position 74 of SEQ ID NO: 2, SEQ ID NO: 4, or SEQ ID NO: 8.

17. The EPO polypeptide of any one of claims 1 to 16 comprising:
a) asparagine at a position corresponding to position 99 of SEQ ID NO: 1, SEQ ID NO: 3, or SEQ ID NO: 7, any amino acid except proline at a position corresponding to position 100 of SEQ ID NO: 1, SEQ ID NO: 3, or SEQ ID NO: 7, and serine or threonine at a position corresponding to position 101 of SEQ ID NO: 1, SEQ ID NO: 3, or SEQ ID NO: 7; or
b) asparagine at a position corresponding to position 73 of SEQ ID NO: 2, SEQ ID NO: 4, or SEQ ID NO: 8, any amino acid except proline at a position corresponding to position 74 of SEQ ID NO: 2, SEQ ID NO: 4, or SEQ ID NO: 8, and serine or threonine at a position corresponding to position 75 of SEQ ID NO: 2, SEQ ID NO: 4, or SEQ ID NO: 8.

18. The EPO polypeptide of any one of claims 1 to 17 comprising:
a) asparagine at a position corresponding to position 112 of SEQ ID NO: 1, SEQ ID NO: 3, or SEQ ID NO: 7, any amino acid except proline at a position corresponding to position 113 of SEQ ID NO: 1, SEQ ID NO: 3, or SEQ ID NO: 7, and serine or threonine at a position corresponding to position 114 of SEQ ID NO: 1, SEQ ID NO: 3, or SEQ ID NO: 7; or
b) asparagine at a position corresponding to position 86 of SEQ ID NO: 2, SEQ ID NO: 4, or SEQ ID NO: 8, any amino acid except proline at a position corresponding to position 87 of SEQ ID NO: 2, SEQ ID NO: 4, or SEQ ID NO: 8, and serine or threonine at a position corresponding to position 88 of SEQ ID NO: 2, SEQ ID NO: 4, or SEQ ID NO: 8.

19. The EPO polypeptide of any one of claims 1 to 18 comprising:
a) asparagine at a position corresponding to position 113 of SEQ ID NO: 1, SEQ ID NO: 3, or SEQ ID NO: 7, any amino acid except proline at a position corresponding to position 114 of SEQ ID NO: 1, SEQ ID NO: 3, or SEQ ID NO: 7, and serine or threonine at a position corresponding to position 115 of SEQ ID NO: 1, SEQ ID NO: 3, or SEQ ID NO: 7; or
b) asparagine at a position corresponding to position 87 of SEQ ID NO: 2, SEQ ID NO: 4, or SEQ ID NO: 8, any amino acid except proline at a position corresponding to position 88 of SEQ ID NO: 2, SEQ ID NO: 4, or SEQ ID NO: 8, and serine or threonine at a position corresponding to position 89 of SEQ ID NO: 2, SEQ ID NO: 4, or SEQ ID NO: 8.

20. The EPO polypeptide of any one of claims 1 to 19 comprising:
a) an asparagine at a position corresponding to position 114 of SEQ ID NO: 1, SEQ ID NO: 3, or SEQ ID NO: 7, any amino acid except proline at a position corresponding to position 115 of SEQ ID NO: 1, SEQ ID NO: 3, or SEQ ID NO: 7, and serine or threonine at a position corresponding to position 116 of SEQ ID NO: 1, SEQ ID NO: 3, or SEQ ID NO: 7, and optionally any amino acid except proline at a position corresponding to position 113 of SEQ ID NO: 1, SEQ ID NO: 3, or SEQ ID NO: 7; or
b) an asparagine at a position corresponding to position 88 of SEQ ID NO: 2, SEQ ID NO: 4, or SEQ ID NO: 8, any amino acid except proline at a position corresponding to position 89 of SEQ ID NO: 2, SEQ ID NO: 4, or SEQ ID NO: 8, and serine or threonine at a position corresponding to position 90 of SEQ ID NO: 2, SEQ ID NO: 4, or SEQ ID NO: 8, and optionally any amino acid except proline at a position corresponding to position 87 of SEQ ID NO: 2, SEQ ID NO: 4, or SEQ ID NO: 8.

21. The EPO polypeptide of any one of claims 1 to 20 comprising:
a) asparagine at a position corresponding to position 115 of SEQ ID NO: 1, SEQ ID NO: 3, SEQ ID NO: 7, any amino acid except proline at a position corresponding to position 116 of SEQ ID NO: 1, SEQ ID NO: 3, or SEQ ID NO: 7, and serine or threonine at a position corresponding to position 117 of SEQ ID NO: 1, SEQ ID NO: 3, or SEQ ID NO: 7; or
b) asparagine at a position corresponding to position 89 of SEQ ID NO: 2, SEQ ID NO: 4, or SEQ ID NO: 8, any amino acid except proline at a position corresponding to position 90 of SEQ ID NO: 2, SEQ ID NO: 4, or SEQ ID NO: 8, and serine or threonine at a position corresponding to position 91 of SEQ ID NO: 2, SEQ ID NO: 4, or SEQ ID NO: 8.

22. The EPO polypeptide of any one of claims 1 to 21 comprising:
a) asparagine at a position corresponding to position 116 of SEQ ID NO: 1, SEQ ID NO: 3, or SEQ ID NO: 7, any amino acid except proline at a position corresponding to position 117 of SEQ ID NO: 1, SEQ ID NO: 3, or SEQ ID NO: 7, and serine or threonine at a position corresponding to position 118 of SEQ ID NO: 1, SEQ ID NO: 3, or SEQ ID NO: 7; or
b) asparagine at a position corresponding to position 90 of SEQ ID NO: 2, SEQ ID NO: 4, or SEQ ID NO: 8, any amino acid except proline at a position corresponding to position 91 of SEQ ID NO: 2, SEQ ID NO: 4, or SEQ ID NO: 8, and serine or threonine at a position corresponding to position 92 of SEQ ID NO: 2, SEQ ID NO: 4, or SEQ ID NO: 8.

23. The EPO polypeptide of any one of claims 1 to 22 comprising:
a) asparagine at a position corresponding to position 137 of SEQ ID NO: 1, SEQ ID NO: 3, or SEQ ID NO: 7, any amino acid except proline at a position corresponding to position 138 of SEQ ID NO: 1, SEQ ID NO: 3, or SEQ ID NO: 7, and serine or threonine at a position corresponding to position 139 of SEQ ID NO: 1, or SEQ ID NO: 3, or SEQ ID NO: 7; or
b) asparagine at a position corresponding to position 111 of SEQ ID NO: 2, SEQ ID NO: 4, or SEQ ID NO: 8, any amino acid except proline at a position corresponding to position 112 of SEQ ID NO: 2, SEQ ID NO: 4, or SEQ ID NO: 8, and serine or threonine at a position corresponding to position 113 of SEQ ID NO: 2, SEQ ID NO: 4, or SEQ ID NO: 8.

24. The EPO polypeptide of any one of claims 1 to 23 comprising:
a) asparagine at a position corresponding to position 138 of SEQ ID NO: 1, SEQ ID NO: 3, or SEQ ID NO: 7, any amino acid except proline at a position corresponding to position 139 of SEQ ID NO: 1, SEQ ID NO: 3, or SEQ ID NO: 7, and serine or threonine at a position corresponding to position 140 of SEQ ID NO: 1, SEQ ID NO: 3, or SEQ ID NO: 7; or
b) asparagine at a position corresponding to position 112 of SEQ ID NO: 2, SEQ ID NO: 4, or SEQ ID NO: 8, any amino acid except proline at a position corresponding to position 113 of SEQ ID NO: 2, SEQ ID NO: 4, or SEQ ID NO: 8, and serine or threonine at a position corresponding to position 114 of SEQ ID NO: 2, SEQ ID NO: 4, or SEQ ID NO: 8.

25. The EPO polypeptide of any one of claims 1 to 24 comprising:
a) asparagine at a position corresponding to position 140 of SEQ ID NO: 1, SEQ ID NO: 3, or SEQ ID NO: 7, any amino acid except proline at a position corresponding to position 141 of SEQ ID NO: 1, SEQ ID NO: 3, or SEQ ID NO: 7, and serine or threonine at a position corresponding to position 142 of SEQ ID NO: 1, SEQ ID NO: 3, or SEQ ID NO: 7; or
b) asparagine at a position corresponding to position 114 of SEQ ID NO: 2, SEQ ID NO: 4, or SEQ ID NO: 8, any amino acid except proline at a position corresponding to position 115 of SEQ ID NO: 2, SEQ ID NO: 4, or SEQ ID NO: 8, and serine or threonine at a position corresponding to position 116 of SEQ ID NO: 2, SEQ ID NO: 4, or SEQ ID NO: 8.

26. The EPO polypeptide of any one of claims 1 to 25 comprising:
a) asparagine at a position corresponding to position 141 of SEQ ID NO: 1, SEQ ID NO: 3, or SEQ ID NO: 7, any amino acid except proline at a position corresponding to position 142 of SEQ ID NO: 1, SEQ ID NO: 3, or SEQ ID NO: 7, and serine or threonine at a position corresponding to position 143 of SEQ ID NO: 1, SEQ ID NO: 3, or SEQ ID NO: 7; or
b) asparagine at a position corresponding to position 115 of SEQ ID NO: 2, SEQ ID NO: 4, or SEQ ID NO: 8, any amino acid except proline at a position corresponding to position 116 of SEQ ID NO: 2, SEQ ID NO: 4, or SEQ ID NO: 8, and serine or threonine at a position corresponding to position 117 of SEQ ID NO: 2, SEQ ID NO: 4, or SEQ ID NO: 8.

27. The EPO polypeptide of any one of claims 1 to 26 comprising:
a) asparagine at a position corresponding to position 142 of SEQ ID NO: 1, SEQ ID NO: 3, or SEQ ID NO: 7, any amino acid except proline at a position corresponding to position 143 of SEQ ID NO: 1, SEQ ID NO: 3, or SEQ ID NO: 7, and serine or threonine at a position corresponding to position 144 of SEQ ID NO: 1, SEQ ID NO: 3, or SEQ ID NO: 7; or
b) asparagine at a position corresponding to position 116 of SEQ ID NO: 2, SEQ ID NO: 4, or SEQ ID NO: 8, any amino acid except proline at a position corresponding to position 117 of SEQ ID NO: 2, SEQ ID NO: 4, or SEQ ID NO: 8, and serine or threonine at a position corresponding to position 118 of SEQ ID NO: 2, SEQ ID NO: 4, or SEQ ID NO: 8.

28. The EPO polypeptide of any one of claims 1 to 27 comprising:
a) asparagine at a position corresponding to position 143 of SEQ ID NO: 1, SEQ ID NO: 3, or SEQ ID NO: 7, any amino acid except proline at a position corresponding to position 144 of SEQ ID NO: 1, SEQ ID NO: 3, or SEQ ID NO: 7, and serine or threonine at a position corresponding to position 145 of SEQ ID NO: 1, SEQ ID NO: 3, or SEQ ID NO: 7; or
b) asparagine at a position corresponding to position 117 of SEQ ID NO: 2, SEQ ID NO: 4, or SEQ ID NO: 8, any amino acid except proline at a position corresponding to position 118 of SEQ ID NO: 2, SEQ ID NO: 4, or SEQ ID NO: 8, and serine or threonine at a position corresponding to position 119 of SEQ ID NO: 2, SEQ ID NO: 4, or SEQ ID NO: 8.

29. The EPO polypeptide of any one of claims 1 to 28 comprising:
a) asparagine at a position corresponding to position 144 of SEQ ID NO: 1, SEQ ID NO: 3, or SEQ ID NO: 7, any amino acid except proline at a position corresponding to position 145 of SEQ ID NO: 1, SEQ ID NO: 3, or SEQ ID NO: 7, and serine or threonine at a position corresponding to position 146 of SEQ ID NO: 1, SEQ ID NO: 3, or SEQ ID NO: 7; or
a) asparagine at a position corresponding to position 118 of SEQ ID NO: 2, SEQ ID NO: 4, or SEQ ID NO: 8, any amino acid except proline at a position corresponding to position 119 of SEQ ID NO: 2, SEQ ID NO: 4, or SEQ ID NO: 8, and serine or threonine at a position corresponding to position 120 of SEQ ID NO: 2, SEQ ID NO: 4, or SEQ ID NO: 8.

30. The EPO polypeptide of any one of claims 1 to 29 comprising:
a) asparagine at a position corresponding to position 145 of SEQ ID NO: 1, SEQ ID NO: 3, or SEQ ID NO: 7, any amino acid except proline at a position corresponding to position 146 of SEQ ID NO: 1, SEQ ID NO: 3, or SEQ ID NO: 7, and serine or threonine at a position corresponding to position 147 of SEQ ID NO: 1, SEQ ID NO: 3, or SEQ ID NO: 7; or
b) asparagine at a position corresponding to position 119 of SEQ ID NO: 2, SEQ ID NO: 4, or SEQ ID NO: 8, any amino acid except proline at a position corresponding to position 120 of SEQ ID NO: 2, SEQ ID NO: 4, or SEQ ID NO: 8, and serine or threonine at a position corresponding to position 121 of SEQ ID NO: 2, SEQ ID NO: 4, or SEQ ID NO: 8.

31. The EPO polypeptide of any one of claims 1 to 30 comprising:
a) asparagine at a position corresponding to position 146 of SEQ ID NO: 1, SEQ ID NO: 3, or SEQ ID NO: 7, any amino acid except proline at a position corresponding to position 147 of SEQ ID NO: 1, SEQ ID NO: 3, or SEQ ID NO: 7, and serine or threonine at a position corresponding to position 148 of SEQ ID NO: 1, SEQ ID NO: 3, or SEQ ID NO: 7; or
b) asparagine at a position corresponding to position 120 of SEQ ID NO: 2, SEQ ID NO: 4, or SEQ ID NO: 8, any amino acid except proline at a position corresponding to position 121 of SEQ ID NO: 2, SEQ ID NO: 4, or SEQ ID NO: 8, and serine or threonine at a position corresponding to position 122 of SEQ ID NO: 2, SEQ ID NO: 4, or SEQ ID NO: 8.

32. The EPO polypeptide of any one of claims 1 to 31 comprising:
a) asparagine at a position corresponding to position 147 of SEQ ID NO: 1, SEQ ID NO: 3, or SEQ ID NO: 7, any amino acid except proline at a position corresponding to position 148 of SEQ ID NO: 1, SEQ ID NO: 3, or SEQ ID NO: 7, and serine or threonine at a position corresponding to position 149 of SEQ ID NO: 1, SEQ ID NO: 3, or SEQ ID NO: 7; or
b) asparagine at a position corresponding to position 121 of SEQ ID NO: 2, SEQ ID NO: 4, or SEQ ID NO: 8, any amino acid except proline at a position corresponding to position 122 of SEQ ID NO: 2, SEQ ID NO: 4, or SEQ ID NO: 8, and serine or threonine at a position corresponding to position 123 of SEQ ID NO: 2, SEQ ID NO: 4, or SEQ ID NO: 8.

33. The EPO polypeptide of any one of claims 1 to 32 comprising:
a) asparagine at a position corresponding to position 148 of SEQ ID NO: 1, SEQ ID NO: 3, or SEQ ID NO: 7, any amino acid except proline at a position corresponding to position 149 of SEQ ID NO: 1, SEQ ID NO: 3, or SEQ ID NO: 7, and serine or threonine at a position corresponding to position 150 of SEQ ID NO: 1, SEQ ID NO: 3, or SEQ ID NO: 7; or
b) asparagine at a position corresponding to position 122 of SEQ ID NO: 2, SEQ ID NO: 4, or SEQ ID NO: 8, any amino acid except proline at a position corresponding to position 123 of SEQ ID NO: 2, SEQ ID NO: 4, or SEQ ID NO: 8, and serine or threonine at a position corresponding to position 124 of SEQ ID NO: 2, SEQ ID NO: 4, or SEQ ID NO: 8.

34. The EPO polypeptide of any one of claims 1 to 33 comprising:
a) asparagine at a position corresponding to position 149 of SEQ ID NO: 1, SEQ ID NO: 3, or SEQ ID NO: 7, any amino acid except proline at a position corresponding to position 150 of SEQ ID NO: 1, SEQ ID NO: 3, or SEQ ID NO: 7, and serine or threonine at a position corresponding to position 151 of SEQ ID NO: 1, SEQ ID NO: 3, or SEQ ID NO: 7; or
b) asparagine at a position corresponding to position 123 of SEQ ID NO: 2, SEQ ID NO: 4, or SEQ ID NO: 8, any amino acid except proline at a position corresponding to position 124 of SEQ ID NO: 2, SEQ ID NO: 4, or SEQ ID NO: 8, and serine or threonine at a position corresponding to position 125 of SEQ ID NO: 2, SEQ ID NO: 4, or SEQ ID NO: 8.

35. The EPO polypeptide of any one of claims 1 to 34 comprising:
a) asparagine at a position corresponding to position 150 of SEQ ID NO: 1, SEQ ID NO: 3, or SEQ ID NO: 7, any amino acid except proline at a position corresponding to position 151 of SEQ ID NO: 1, SEQ ID NO: 3, or SEQ ID NO: 7, and serine or threonine at a position corresponding to position 152 of SEQ ID NO: 1, SEQ ID NO: 3, or SEQ ID NO: 7; or
b) asparagine at a position corresponding to position 124 of SEQ ID NO: 2, SEQ ID NO: 4, or SEQ ID NO: 8, any amino acid except proline at a position corresponding to position 125 of SEQ ID NO: 2, SEQ ID NO: 4, or SEQ ID NO: 8, and serine or threonine at a position corresponding to position 126 of SEQ ID NO: 2, SEQ ID NO: 4, or SEQ ID NO: 8.

36. The EPO polypeptide of any one of claims 1 to 35 comprising:
a) asparagine at a position corresponding to position 161 of SEQ ID NO: 1, SEQ ID NO: 3, or SEQ ID NO: 7, any amino acid except proline at a position corresponding to position 162 of SEQ ID NO: 1, SEQ ID NO: 3, or SEQ ID NO: 7, and serine or threonine at a position corresponding to position 163 of SEQ ID NO: 1, SEQ ID NO: 3, or SEQ ID NO: 7; or
b) asparagine at a position corresponding to position 135 of SEQ ID NO: 2, SEQ ID NO: 4, or SEQ ID NO: 8, any amino acid except proline at a position corresponding to position 136 of SEQ ID NO: 2, SEQ ID NO: 4, or SEQ ID NO: 8, and serine or threonine at a position corresponding to position 137 of SEQ ID NO: 2, SEQ ID NO: 4, or SEQ ID NO: 8.

37. The EPO polypeptide of any one of claims 1 to 36 comprising:
a) asparagine at a position corresponding to position 162 of SEQ ID NO: 1, SEQ ID NO: 3, or SEQ ID NO: 7, any amino acid except proline at a position corresponding to position 163 of SEQ ID NO: 1, SEQ ID NO: 3, or SEQ ID NO: 7, and serine or threonine at a position corresponding to position 164 of SEQ ID NO: 1, SEQ ID NO: 3, or SEQ ID NO: 7; or
b) asparagine at a position corresponding to position 136 of SEQ ID NO: 2, SEQ ID NO: 4, or SEQ ID NO: 8, any amino acid except proline at a position corresponding to position 137 of SEQ ID NO: 2, SEQ ID NO: 4, or SEQ ID NO: 8, and serine or threonine at a position corresponding to position 138 of SEQ ID NO: 2, SEQ ID NO: 4, or SEQ ID NO: 8.

38. The EPO polypeptide of any one of claims 1 to 37 comprising:
a) asparagine at a position corresponding to position 184 of SEQ ID NO: 1, SEQ ID NO: 3, or SEQ ID NO: 7, any amino acid except proline at a position corresponding to position 185 of SEQ ID NO: 1, SEQ ID NO: 3, or SEQ ID NO: 7, and serine or threonine at a position corresponding to position 186 of SEQ ID NO: 1, SEQ ID NO: 3, or SEQ ID NO: 7; or
b) asparagine at a position corresponding to position 158 of SEQ ID NO: 2, SEQ ID NO: 4, or SEQ ID NO: 8, any amino acid except proline at a position corresponding to position 159 of SEQ ID NO: 2, SEQ ID NO: 4, or SEQ ID NO: 8, and serine or threonine at a position corresponding to position 160 of SEQ ID NO: 2, SEQ ID NO: 4, or SEQ ID NO: 8.

39. The EPO polypeptide of any one of claims 1 to 38 comprising:
a) asparagine at a position corresponding to position 186 of SEQ ID NO: 1, SEQ ID NO: 3, or SEQ ID NO: 7, any amino acid except proline at a position corresponding to position 187 of SEQ ID NO: 1, SEQ ID NO: 3, or SEQ ID NO: 7, and serine or threonine at a position corresponding to position 188 of SEQ ID NO: 1, SEQ ID NO: 3, or SEQ ID NO: 7; or
b) asparagine at a position corresponding to position 162 of SEQ ID NO: 2, SEQ ID NO: 4, or SEQ ID NO: 8, any amino acid except proline at a position corresponding to position 163 of SEQ ID NO: 2, SEQ ID NO: 4, or SEQ ID NO: 8, and serine or threonine at a position corresponding to position 164 of SEQ ID NO: 2, SEQ ID NO: 4, or SEQ ID NO: 8.

40. The EPO polypeptide of any one of claims 1 to 39 comprising the amino acid sequence of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121.

41. An EPO polypeptide comprising the amino acid sequence of SEQ ID NO: 1, SEQ ID NO: 2, SEQ ID NO: 3, SEQ ID NO: 4, SEQ ID NO: 7, or SEQ ID NO: 8 except for the presence of at least one cysteine not present in SEQ ID NO: 1, SEQ ID NO: 2, SEQ ID NO: 3, SEQ ID NO: 4, SEQ ID NO: 7, or SEQ ID NO: 8.

42. The EPO polypeptide of any one of claims 1 to 41 comprising:
a) a cysteine at position 45, 48, 49, 68, 86, 90, 92 120, 143, 144, and/or 172 of SEQ ID NO: 1, SEQ ID NO: 3, or SEQ ID NO: 7; or
b) a cysteine at position 19, 22, 23, 42, 60, 64, 66, 94, 117, 118, and/or 146 of SEQ ID NO: 2, SEQ ID NO: 4, or SEQ ID NO: 8.

43. The EPO polypeptide of claim 41 or claim 42 comprising:
a) a cysteine at position 45 and 172 of SEQ ID NO: 1, SEQ ID NO: 3, or SEQ ID NO: 7; or
b) a cysteine at position 19 and 146 of SEQ ID NO: 2, SEQ ID NO: 4, or SEQ ID NO: 8.

44. The EPO polypeptide of any one of claims 41 to 43 comprising:
a) a cysteine at position 48 and 120 of SEQ ID NO: 1, SEQ ID NO: 3, or SEQ ID NO: 7; or
b) a cysteine at position 22 and 94 of SEQ ID NO: 2, SEQ ID NO: 4, or SEQ ID NO: 8.

45. The EPO polypeptide of any one of claims 41 to 44 comprising:
a) a cysteine at position 49 and 172 of SEQ ID NO: 1, SEQ ID NO: 3, or SEQ ID NO: 7; or
b) a cysteine at position 23 and 146 of SEQ ID NO: 2, SEQ ID NO: 4, or SEQ ID NO: 8.

46. The EPO polypeptide of any one of claims 41 to 45 comprising:
a) a cysteine at position 68 and 92 of SEQ ID NO: 1, SEQ ID NO: 3, or SEQ ID NO: 7; or
b) a cysteine at position 42 and 66 of SEQ ID NO: 2, SEQ ID NO: 4, or SEQ ID NO: 8.

47. The EPO polypeptide of any one of claims 41 to 46 comprising:
a) a cysteine at position 90 and 144 of SEQ ID NO: 1, SEQ ID NO: 3, or SEQ ID NO: 7; or
b) a cysteine at position 64 and 118 of SEQ ID NO: 2, SEQ ID NO: 4, or SEQ ID NO: 8.

48. The EPO polypeptide of any one of claims 41 to 47 comprising:
a) a cysteine at position 86 and 143 of SEQ ID NO: 1, SEQ ID NO: 3, or SEQ ID NO: 7; or
b) a cysteine at position 60 and 117 of SEQ ID NO: 2, SEQ ID NO: 4, or SEQ ID NO: 8.

49. The EPO polypeptide of any one of claims 1 to 48 comprising the amino acid sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, or SEQ ID NO: 32.

50. The EPO polypeptide of any one of claims 1 to 49 comprising an amino acid other than a cysteine at a position corresponding to position 165 of SEQ ID NO: 7 or at a position corresponding to position 139 of SEQ ID NO: 8.

51. An EPO polypeptide comprising the amino acid sequence of SEQ ID NO: 7 or SEQ ID NO: 8 except for the presence of an amino acid other than a cysteine at position 165 of SEQ ID NO: 7 or at position 139 of SEQ ID NO: 8.

52. The EPO polypeptide of claim 50 or 51, wherein the amino acid other than a cysteine is a threonine, a serine, or an alanine.

53. The EPO polypeptide of any one of claims 1 to 52, wherein the N-linked glycosylation site comprises an amino acid derivative.

54. The EPO polypeptide of claim 53, wherein the amino acid derivative is an asparagine derivative, a serine derivative, or a threonine derivative.

55. The EPO polypeptide of any one of claims 1 to 54, wherein the EPO polypeptide is glycosylated.

56. The EPO polypeptide of any one of claims 1 to 55 comprising at least one glycan moiety attached to the N-linked glycosylation site.

57. The EPO polypeptide of any one of claims 1 to 56, wherein the EPO polypeptide is PEGylated.

58. The EPO polypeptide of any one of claims 1 to 57, wherein the EPO polypeptide is PEGylated at a glycan.

59. The EPO polypeptide of any one of claims 1 to 58, wherein the EPO polypeptide is PEGylated at a primary amine.

60. The EPO polypeptide of any one of claims 1 to 59, wherein the EPO polypeptide is PEGylated at the N-terminal alpha-amine.

61. A contiguous polypeptide comprising the EPO polypeptide of any one of claims 1 to 60, wherein the contiguous polypeptide comprises an IgG Fc polypeptide.

62. The contiguous polypeptide of claim 61, wherein the IgG Fc polypeptide is a wild-type IgG Fc polypeptide.

63. The contiguous polypeptide of claim 61, wherein the IgG Fc polypeptide is a variant IgG Fc polypeptide.

64. The contiguous polypeptide of any one of claims 60 to 63, wherein the IgG Fc polypeptide is a variant IgG Fc polypeptide comprising:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide; and/or
c) at least one amino acid modification relative to a wild-type IgG Fc polypeptide of a companion animal species, wherein the variant IgG Fc polypeptide has reduced binding affinity to CD16 relative to the wild-type IgG Fc polypeptide.

65. The contiguous polypeptide of any one of the claims 60 to 64, wherein the variant IgG Fc polypeptide binds to C1q and/or CD16 with a dissociation constant (K_{d}) of greater than 5×10⁻⁶ M, greater than 1×10⁻⁵M, greater than 5×10⁻⁵M, greater than 1×10⁻⁴ M, greater than 5×10⁻⁴ M, or greater than 1×10⁻³M, as measured by biolayer interferometry.

66. The contiguous polypeptide of any one of the claims 60 to 65, wherein the variant IgG Fc polypeptide binds to Protein A with a dissociation constant (K_{d}) of less than 5×10⁻⁶ M, less than 1×10⁻⁶ M, less than 5×10⁻⁷ M, less than 1×10⁻⁷M, less than 5×10⁻⁸M, less than 1×10⁻⁸ M, less than 5×10⁻⁹M, less than 1×10⁻⁹ M, less than 5×10⁻¹⁰ M, less than 1×10⁻¹⁰ M, less than 5×10⁻¹¹ M, less than 1×10⁻¹¹M, less than 5×10⁻¹² M, or less than 1×10⁻¹²M, as measured by biolayer interferometry.

67. The contiguous polypeptide of any one of the claims 60 to 66, wherein the companion animal species is canine, feline, or equine.

68. The contiguous polypeptide of any one of the claims 60 to 67, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1a Fc, IgG1b Fc, or IgG2 Fc.

69. The contiguous polypeptide of any one of the claims 60 to 68, wherein the variant IgG Fc polypeptide comprises:
a) an amino acid substitution at a position corresponding to position 21, position 23, position 25, position 80, position 205, and/or position 207 of SEQ ID NO: 53;
b) an amino acid substitution at a position corresponding to position 21, position 23, and/or position 24 of SEQ ID NO: 56;
c) an amino acid substitution at a position corresponding to position 21, position 23, position 25, position 80, and/or position 207 of SEQ ID NO: 58;
d) an amino acid substitution at a position corresponding to position 15 and/or position 203 of SEQ ID NO: 88;
e) an amino acid substitution at a position corresponding to position 199 and/or position 200 of SEQ ID NO: 92; and/or
f) an amino acid substitution at a position corresponding to position 199, position 200, position 201, and/or position 202 of SEQ ID NO: 93.

70. The contiguous polypeptide of any one of the claims 60 to 69, wherein the variant IgG Fc polypeptide comprises:
a) an amino acid substitution at position 21, position 23, position 25, position 80, position 205, and/or position 207 of SEQ ID NO: 53;
b) an amino acid substitution at position 21, position 23, and/or position 24 of SEQ ID NO: 56;
c) an amino acid substitution at position 21, position 23, position 25, position 80, and/or position 207 of SEQ ID NO: 58;
d) an amino acid substitution at position 15 and/or position 203 of SEQ ID NO: 88;
e) an amino acid substitution at position 199 and/or position 200 of SEQ ID NO: 92; and/or
f) an amino acid substitution at position 199, position 200, position 201, and/or position 202 of SEQ ID NO: 93.

71. The contiguous polypeptide of any one of the claims 60 to 70, wherein the variant IgG Fc polypeptide comprises:
a) a threonine at a position corresponding to position 21, a leucine at a position corresponding to position 23, an alanine at a position corresponding to position 25, a glycine at a position corresponding to position 80, an alanine at a position corresponding to position 205, and/or a histidine at a position corresponding to position 207 of SEQ ID NO: 53;
b) a threonine at a position corresponding to position 21, a leucine at a position corresponding to position 23, and/or an isoleucine at a position corresponding to position 24 of SEQ ID NO: 56;
c) a threonine at a position corresponding to position 21, a leucine at a position corresponding to position 23, an alanine at a position corresponding to position 25, a glycine at a position corresponding to position 80, and/or a histidine at a position corresponding to position 207 of SEQ ID NO: 58;
d) a threonine or a valine at a position corresponding to position 15 and/or a tyrosine or a valine at a position corresponding to position 203 of SEQ ID NO: 88;
e) a leucine at a position corresponding to position 199 and/or a histidine at a position corresponding to position 200 of SEQ ID NO: 92; and/or
f) a leucine at a position corresponding to position 199, a histidine at a position corresponding to position 200, an asparagine at a position corresponding to position 201, and/or a histidine at a position corresponding to position 202 of SEQ ID NO: 93.

72. The contiguous polypeptide of any one of the claims 60 to 71, wherein the variant IgG Fc polypeptide comprises:
a) a threonine at position 21, a leucine at position 23, an alanine at position 25, a glycine at position 80, an alanine at position 205, and/or a histidine at position 207 of SEQ ID NO: 53;
b) a threonine at position 21, a leucine at position 23, and/or an isoleucine at position 24 of SEQ ID NO: 56;
c) a threonine at a position 21, a leucine at position 23, an alanine at position 25, a glycine at position 80, and/or a histidine at position 207 of SEQ ID NO: 58;
d) a threonine or a valine at position 15, and/or a tyrosine or a valine at position 203 of SEQ ID NO: 88;
e) a leucine at position 199 and/or a histidine at position 200 of SEQ ID NO: 92; and/or
f) a leucine at position 199, a histidine at position 200, an asparagine at position 201, and/or a histidine at position 202 of SEQ ID NO: 93.

73. The contiguous polypeptide of any one of the claims 60 to 72, wherein the variant IgG Fc polypeptide comprises:
a) an amino acid substitution at a position corresponding to position 93 of SEQ ID NO: 54 or SEQ ID NO: 56;
b) an amino acid substitution at a position corresponding to position 87 of SEQ ID NO: 87, SEQ ID NO: 90, SEQ ID NO: 91, or SEQ ID NO: 94; or
c) an amino acid substitution at a position corresponding to position 198 of SEQ ID NO: 103, SEQ ID NO: 104, SEQ ID NO: 105, or SEQ ID NO: 106.

74. The contiguous polypeptide of any one of the claims 60 to 73, wherein the variant IgG Fc polypeptide comprises:
a) an amino acid substitution at position 93 of SEQ ID NO: 54 or SEQ ID NO: 56;
b) an amino acid substitution at position 87 of SEQ ID NO: 87, SEQ ID NO: 90, SEQ ID NO: 91, or SEQ ID NO: 94; or
c) an amino acid substitution at position 198 of SEQ ID NO: 103, SEQ ID NO: 104, SEQ ID NO: 105, or SEQ ID NO: 106.

75. The contiguous polypeptide of any one of the claims 60 to 74, wherein the variant IgG Fc polypeptide comprises:
a) an arginine at a position corresponding to position 93 of SEQ ID NO: 54 or SEQ ID NO: 56;
b) a serine at a position corresponding to position 87 of SEQ ID NO: 87, SEQ ID NO: 90, SEQ ID NO: 91, or SEQ ID NO: 94; or
c) an alanine at a position corresponding to position 198 of SEQ ID NO: 103, SEQ ID NO: 104, SEQ ID NO: 105, or SEQ ID NO: 106.

76. The contiguous polypeptide of any one of the claims 60 to 75, wherein the variant IgG Fc polypeptide comprises:
a) an arginine at position 93 of SEQ ID NO: 54 or SEQ ID NO: 56;
b) a serine at position 87 of SEQ ID NO: 87, SEQ ID NO: 90, SEQ ID NO: 91, or SEQ ID NO: 94; or
c) an alanine at position 198 of SEQ ID NO: 103, SEQ ID NO: 104, SEQ ID NO: 105, or SEQ ID NO: 106.

77. The contiguous polypeptide of any one of the claims 60 to 76, wherein the variant IgG Fc polypeptide comprises:
a) an amino acid substitution at a position corresponding to position 5, position 38, position 39, position 97, and/or position 98 of SEQ ID NO: 54; or
b) an amino acid substitution at a position corresponding to position 5, position 38, position 39, position 97, and/or position 98 of SEQ ID NO: 56.

78. The contiguous polypeptide of any one of the claims 60 to 77, wherein the variant IgG Fc polypeptide comprises:
a) an amino acid substitution at position 5, position 38, position 39, position 97, and/or position 98 of SEQ ID NO: 54; or
b) an amino acid substitution at position 5, position 38, position 39, position 97, and/or position 98 of SEQ ID NO: 56.

79. The contiguous polypeptide of any one of the claims 60 to 78, wherein the variant IgG Fc polypeptide comprises:
a) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 54; or
b) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 56.

80. The contiguous polypeptide of any one of the claims 60 to 80, wherein the variant IgG Fc polypeptide comprises:
a) a proline at position 5, a glycine at position 38, an arginine at position 39, an isoleucine at position 97, and/or a glycine at position 98 of SEQ ID NO: 54; or
b) a proline at position 5, a glycine at position 38, an arginine at position 39, an isoleucine at position 97, and/or a glycine at position 98 of SEQ ID NO: 56.

81. The contiguous polypeptide of any one of claims 60 to 81, wherein the variant IgG Fc polypeptide comprises the amino acid sequence of SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, or SEQ ID NO: 111.

82. A composition comprising a plurality of EPO polypeptides of any one of claims 1 to 81 having a range of isoelectric points of from about 1 to about 3.5, of from about 1.5 to about 3.5, of from about 2 to about 3.5, of from about 2.5 to about 3.5, of from about 3 to about 3.5, of about 3.5 or less, or of about 3 or less, as determined by isoelectric focusing.

83. A composition comprising a plurality of EPO polypeptides of any one of claims 1 to 81 having a range of isoelectric points of from about 3.5 to about 6, of from about 4 to about 6, of from about 4.5 to about 6, of from about 5 to about 6, of from about 5.5 to about 6, of from about 3.5 to about 5, of from about 4 to about 5, of from about 4.5 to about 5, of about 3.5 or greater, of about 4 or greater, or of about 4.5 or greater, as determined by isoelectric focusing.

84. A combination comprising the composition of claim 82 and the composition of claim 83.

85. An isolated nucleic acid encoding the EPO polypeptide of any one of claims 1 to 81.

86. The nucleic acid of claim 85, wherein the nucleic acid comprises a regulatory sequence.

87. The nucleic acid of claim 86, wherein the regulatory sequence is a constitutive promoter; an inducible regulatory sequence, such as a tetracycline response element or a hypoxia-inducible promoter; a tissue specific promoter; an enhancer; a silencer; or encodes a micro RNA or transcription factor.

88. An isolated nucleic acid encoding an EPO polypeptide comprising the amino acid sequence of SEQ ID NO: 1, SEQ ID NO: 2, SEQ ID NO: 3, or SEQ ID NO: 4; and a heterologous regulatory sequence, wherein the heterologous regulatory sequence is not a constitutive promoter.

89. The nucleic acid of claim 64, wherein the heterologous regulatory sequence is an inducible regulatory sequence, such as a tetracycline response element or a hypoxia-inducible promoter; a tissue specific promoter; an enhancer; a silencer; or encodes a micro RNA or transcription factor.

90. A vector comprising the nucleic acid of any one of claims 86 to 89.

91. The vector of claim 90, wherein the vector is a viral vector or a bacterial vector.

92. The vector of claim 90 or claim 91, wherein the vector is a retroviral vector, a herpesviral vector, an adenoviral vector, an adeno-associated viral vector, or a pox viral vector.

93. An expression system comprising a first vector comprising a nucleic acid encoding the EPO polypeptide of any one of claims 1 to 81; and a second vector comprising a regulatory sequence.

94. An expression system comprising a first vector comprising a nucleic acid encoding an EPO polypeptide comprising the amino acid sequence of SEQ ID NO: 1, SEQ ID NO: 2, SEQ ID NO: 3, or SEQ ID NO: 4; and a second vector comprising a regulatory sequence.

95. The expression system of claim 93 or claim 94, wherein the regulatory sequence encodes a micro RNA or transcription factor.

96. The expression system of any one of claims 93 to 95, wherein the first vector and/or second vector is a viral vector or a bacterial vector.

97. The expression system of any one of claims 93 to 96, wherein the first vector and/or second vector is a retroviral vector, a herpesviral vector, an adenoviral vector, an adeno-associated viral vector, or a pox viral vector.

98. A host cell comprising the nucleic acid of any one of claims 85 to 89, the vector of any one of claims 90 to 92, or the expression system of any one of claims 93 to 97.

99. A method of producing a composition comprising EPO polypeptides comprising culturing the host cell of claim 98 and isolating the EPO polypeptides.

100. The method of claim 99, wherein the EPO polypeptides are isolated by column chromatography.

101. The method of claim 99 or claim 100, wherein the EPO polypeptides are isolated by ion exchange column chromatography.

102. The method of any one of claims 99 to 101, wherein the EPO polypeptides are isolated by Capto Butyl column chromatography, cation-exchange column chromatography, or anion-exchange column chromatography.

103. The method of any one of claims 99 to 102, wherein the EPO polypeptides are isolated by mixed-mode column chromatography.

104. The method of any one of claims 99 to 103, wherein the EPO polypeptides are isolated by hydrophobic interaction column chromatography.

105. The method of any one of claims 99 to 104, wherein the EPO polypeptides are isolated by a combination of chromatography columns.

106. The method of any one of claims 99 to 105, wherein the method further comprises inactivating and/or removing viruses.

107. The method of any one of claims 99 to 106, wherein the EPO polypeptides have a range of isoelectric points of from about 1 to about 3.5, of from about 1.5 to about 3.5, of from about 2 to about 3.5, of from about 2.5 to about 3.5, of from about 3 to about 3.5, of about 3.5 or less, or of about 3 or less, as determined by isoelectric focusing.

108. The method of any one of claims 99 to 106, wherein the EPO polypeptides have a range of isoelectric points of from about 3.5 to about 6, of from about 4 to about 6, of from about 4.5 to about 6, of from about 5 to about 6, of from about 5.5 to about 6, of from about 3.5 to about 5, of from about 4 to about 5, of from about 4.5 to about 5, of about 3.5 or greater, of about 4 or greater, or of about 4.5 or greater, as determined by isoelectric focusing.

109. A pharmaceutical composition comprising the EPO polypeptide of any one of claims 1 to 81, the composition of claim 82 or claim 83, the combination of claim 84, the nucleic acid of any one of claims 85 to 89, the vector of any one of claims 90 to 92, or the expression system of any one of claims 93 to 97, and a pharmaceutically acceptable carrier.

110. A pharmaceutical composition comprising the EPO polypeptide of any one of claims 1 to 81, the composition of claim 82 or claim 83, or the combination of claim 84 and a pharmaceutically acceptable carrier, wherein the pharmaceutically acceptable carrier comprises a) sodium phosphate, sodium chloride, and polysorbate 80; b) sodium phosphate, sodium chloride, and polysorbate 20; c) sodium citrate, sodium chloride, and polysorbate 80; or d) sodium citrate, sodium chloride, and polysorbate 20.

111. A pharmaceutical composition comprising the EPO polypeptide of any one of claims 1 to 81, the composition of claim 82 or claim 83, or the combination of claim 84 and a pharmaceutically acceptable carrier, wherein the pharmaceutically acceptable carrier comprises sodium citrate, sodium chloride, polysorbate 80, and m-cresol.

112. A pharmaceutical composition comprising the EPO polypeptide of any one of claims 1 to 81, the composition of claim 82 or claim 83, or the combination of claim 84 and a pharmaceutically acceptable carrier, wherein the pharmaceutically acceptable carrier comprises sodium phosphate, sodium chloride, polysorbate 20, and benzyl alcohol.

113. The pharmaceutical composition of any one of claims 110 to 112, wherein the concentration of sodium chloride is about 140 mM.

114. The pharmaceutical composition of any one of claims 110 to 112, wherein the concentration of sodium phosphate or sodium citrate is about 20 mM.

115. The pharmaceutical composition of any one of claims 110 to 112, wherein the concentration of polysorbate 20 or polysorbate 80 is about 650 nM.

116. The pharmaceutical composition of any one of claims 111, or 113 to 115, wherein the concentration of m-cresol is about 0.2%.

117. The pharmaceutical composition of any one of claims 112 to 116, wherein the concentration of benzyl alcohol is about 1%.

118. The pharmaceutical composition of any one of claims 110 to 117, wherein the pharmaceutically acceptable carrier comprises:
a) sodium phosphate at a concentration of about 20 mM, sodium chloride at a concentration of about 140 mM, polysorbate 80 at a concentration of about 650 nM or
b) sodium phosphate at a concentration of about 20 mM, sodium chloride at a concentration of about 140 mM, polysorbate 20 at a concentration of about 650 nM.

119. The pharmaceutical composition of any one of claims 110 to 118, wherein the pharmaceutically acceptable carrier comprises sodium citrate at a concentration of about 20 mM, sodium chloride at a concentration of about 140 nM, polysorbate 80 at a concentration of about 650 nM, and m-cresol at a concentration of about 0.2%.

120. The pharmaceutical composition of any one of claims 110 to 119, wherein the pharmaceutically acceptable carrier comprises sodium phosphate at a concentration of about 20 mM, sodium chloride at a concentration of about 140 nM, polysorbate 20 at a concentration of about 650 nM, and benzyl alcohol at a concentration of about 1%.

121. A method of delivering an EPO polypeptide to a companion animal species comprising administering the EPO polypeptide of any one of claims 1 to 81, the composition of claim 82 or claim 83, the combination of claim 84, or the pharmaceutical composition of any one of claims 109 to 120 parenterally.

122. A method of delivering an EPO polypeptide to a companion animal species comprising administering the EPO polypeptide of any one of claims 1 to 81, the composition of claim 82 or claim 83, the combination of claim 84, or the pharmaceutical composition of any one of claims 109 to 120 by an intramuscular route, an intraperitoneal route, an intracerebrospinal route, a subcutaneous route, an intra-arterial route, an intrasynovial route, an intrathecal route, or an inhalation route.

123. A method of delivering an isolated nucleic acid encoding an EPO polypeptide to a companion animal species comprising administering the nucleic acid of any one of claims 85 to 89, the vector of any one of claims 90 to 92, or the expression system of any one of claims 93 to 97 parenterally.

124. A method of treating a companion animal species having anemia comprising administering to the companion animal species a therapeutically effective amount of the EPO polypeptide of any one of claims 1 to 81, the composition of claim 82 or 83, the combination of claim 84, or the pharmaceutical composition of any one of claims 109 to 120.

125. A method of treating a companion animal species having anemia, the method comprising administering to the companion animal species a therapeutically effective amount of the nucleic acid of any one of claims 85 to 89, the vector of any one of claims 90 to 92, or the expression system of any one of claims 93 to 97.

126. The method of claim 124 or claim 125, wherein the EPO polypeptide, composition, nucleic acid, vector, expression system, or pharmaceutical composition is administered parenterally.

127. The method of any one of claims 124 to 126, wherein the EPO polypeptide, composition, nucleic acid, vector, expression system, or pharmaceutical composition is administered by an intramuscular route, an intraperitoneal route, an intracerebrospinal route, a subcutaneous route, an intra-arterial route, an intrasynovial route, an intrathecal route, or an inhalation route.

128. The method of any one of claims 121 to 127, wherein the companion animal species is feline, canine, or equine.

129. The method of any one of claims 124 to 128, wherein the anemia is caused by chronic kidney disease, inflammatory bowel disease, or myelodysplasia.

130. The method of any one of claims 121 to 129, wherein the EPO polypeptide is administered in an amount of from about 1 μg/kg body weight to about 10 μg/kg body weight, or about 1 μg/kg body weight to about 5 μg/kg body weight, or about 1 μg/kg body weight, or about 3 μg/kg body weight, or about 5 μg/kg body weight, or about 10 μg/kg body weight.

131. The method of any one of claims 121 to 130, wherein the EPO polypeptide, composition, nucleic acid, vector, expression system, or pharmaceutical composition is administered every 7 to 10 days.

132. The method of any one of claims 121 to 131, wherein the method comprises administering iron dextran.

133. The method of any one of claims 121 to 132, wherein the companion animal species has a baseline hematocrit percentage of from about 15% to about 30%, of from about 15% to about 25%, of from about 20% to about 25%, of from about 25% to about 30%, of below about 15%, of below about 18%, of below about 20%, of below about 25%, of below about 29%, or of below about 30% prior to administration of the EPO polypeptide, composition, nucleic acid, vector, expression system, or pharmaceutical composition.

134. The method of any one of claims 121 to 133, wherein the hematocrit percentage of the companion animal species increases to at least 25%, or at least 26%, or at least 27%, or at least 28%, or at least 29%, or at least 30%, or at least 32%, or at least 35%, or at least 38%, or at least 40%, or at least 42%, or at least 45%, or at least 48% following administration of the EPO polypeptide, composition, nucleic acid, vector, expression system, or pharmaceutical composition.

135. The method of claim 134, wherein the hematocrit percentage of the companion animal species increases to at least 25%, or at least 27%, or at least 30%, or at least 32%, or at least 35% at 1 week, 2 weeks, 3 weeks, 4 weeks, 5 weeks, or 6 weeks after a first administration of the EPO polypeptide, composition, nucleic acid, vector, expression system, or pharmaceutical composition.

136. The method of any one of claims 121 to 135, wherein the body weight of the companion animal species is maintained or increased compared to baseline following administration of the EPO polypeptide, composition, nucleic acid, vector, expression system, or pharmaceutical composition.

137. The method of claim 136, wherein the body weight of the companion animal species is maintained or increased at 1 week, 2 weeks, 3 weeks, 4 weeks, 5 weeks, or 6 weeks after a first administration of the EPO polypeptide, composition, nucleic acid, vector, expression system, or pharmaceutical composition.

138. The method of any one of claims 121 to 137, wherein the level of symmetric dimethylarginine or serum creatine renal biomarker is decreased compared to baseline following administration of the EPO polypeptide, composition, nucleic acid, vector, expression system, or pharmaceutical composition.

139. A method of expressing an EPO polypeptide in a target cell, comprising
a) transferring a nucleic acid, vector, or expression system into the target cell, wherein the nucleic acid, vector, or expression system comprises:
i) a nucleic acid encoding the EPO polypeptide of any one of claims 1 to 81, and
ii) a regulatory sequence; and
b) culturing the cell under conditions supportive for expression of the EPO polypeptide.

140. A method of expressing an EPO polypeptide in a target cell, comprising
a) transferring a nucleic acid, a vector, or an expression system into the target cell, wherein the nucleic acid, vector, or expression system comprises:
i) a nucleic acid encoding an EPO polypeptide having the amino acid sequence of SEQ ID NO: 1, SEQ ID NO: 2, SEQ ID NO: 3, or SEQ ID NO: 4, and
ii) a regulatory sequence, wherein the regulatory sequence is not a constitutive promoter; and
b) culturing the cell under conditions supportive for expression of the EPO polypeptide.

141. The method of claim 139 or claim 140, wherein the regulatory sequence is an inducible regulatory sequence, such as a tetracycline response element or a hypoxia-inducible promoter; a tissue specific promoter; an enhancer; a silencer; or encodes a micro RNA or transcription factor.

142. The method of any one of claims 139 to 141, wherein the vector is a viral vector or a bacterial vector.

143. The method of any one of claims 139 to 142, wherein the vector is a retroviral vector, a herpesviral vector, an adenoviral vector, an adeno-associated viral vector, or a pox viral vector.

144. The method of any one of claims 139 to 143, wherein the cell is a cell of a companion animal species.

145. The method of any one of claims 139 to 144, wherein the cell is located in a living companion animal species.

146. The method of claim 144 or claim 145, wherein the companion animal species is a canine, feline, or equine.

147. A polypeptide comprising an extracellular domain of a canine, equine, or feline erythropoietin receptor (EPOR) polypeptide, wherein the canine, equine, or feline EPOR polypeptide comprises the amino acid sequence of SEQ ID NO: 33, SEQ ID NO: 37, SEQ ID NO: 41, SEQ ID NO: 44, SEQ ID NO: 47, or SEQ ID NO: 50; and a heterologous polypeptide sequence.

148. A polypeptide comprising the amino acid sequence of SEQ ID NO: 34, SEQ ID NO: 35, SEQ ID NO: 38, SEQ ID NO: 39, SEQ ID NO: 42, SEQ ID NO: 43, SEQ ID NO: 45, SEQ ID NO: 46, SEQ ID NO: 48, SEQ ID NO: 49, SEQ ID NO: 51, or SEQ ID NO: 52; and a heterologous polypeptide sequence.

149. A contiguous polypeptide comprising the polypeptide of claim 147 or claim 148, wherein the contiguous polypeptide comprises an IgG Fc polypeptide.

150. The contiguous polypeptide of claim 149, wherein the IgG Fc polypeptide is a wild-type IgG Fc polypeptide.

151. The contiguous polypeptide of claim 149, wherein the IgG Fc polypeptide is a variant IgG Fc polypeptide.

152. The contiguous polypeptide of any one of claims 149 to 151, wherein the IgG Fc polypeptide is a variant IgG Fc polypeptide comprising:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide; and/or
c) at least one amino acid modification relative to a wild-type IgG Fc polypeptide of a companion animal species, wherein the variant IgG Fc polypeptide has reduced binding affinity to CD16 relative to the wild-type IgG Fc polypeptide.

153. The contiguous polypeptide of any one of the claims 149 to 152, wherein the variant IgG Fc polypeptide binds to C1q and/or CD16 with a dissociation constant (K_{d}) of greater than 5×10⁻⁶M, greater than 1×10⁻⁵M, greater than 5×10⁻⁵M, greater than 1×10⁻⁴M, greater than 5×10⁻⁴M, or greater than 1×10⁻³M, as measured by biolayer interferometry.

154. The contiguous polypeptide of any one of the claims 149 to 153, wherein the variant IgG Fc polypeptide binds to Protein A with a dissociation constant (K_{d}) of less than 5×10⁻⁶M, less than 1×10⁻⁶M, less than 5×10⁻⁷ M, less than 1×10⁻⁷M, less than 5×10⁻⁸M, less than 1×10⁻⁸ M, less than 5×10⁻⁹M, less than 1×10⁻⁹ M, less than 5×10⁻¹⁰ M, less than 1×10⁻¹⁰ M, less than 5×10⁻¹¹ M, less than 1×10⁻¹¹M, less than 5×10⁻¹² M, or less than 1×10⁻¹²M, as measured by biolayer interferometry.

155. The contiguous polypeptide of any one of the claims 149 to 154, wherein the companion animal species is canine, feline, or equine.

156. The contiguous polypeptide of any one of the claims 149 to 155, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1a Fc, IgG1b Fc, or IgG2 Fc.

157. The contiguous polypeptide of any one of the claims 149 to 156, wherein the variant IgG Fc polypeptide comprises:
a) an amino acid substitution at a position corresponding to position 21, position 23, position 25, position 80, position 205, and/or position 207 of SEQ ID NO: 53;
b) an amino acid substitution at a position corresponding to position 21, position 23, and/or position 24 of SEQ ID NO: 56;
c) an amino acid substitution at a position corresponding to position 21, position 23, position 25, position 80, and/or position 207 of SEQ ID NO: 58;
d) an amino acid substitution at a position corresponding to position 15 and/or position 203 of SEQ ID NO: 88;
e) an amino acid substitution at a position corresponding to position 199 and/or position 200 of SEQ ID NO: 92; and/or
f) an amino acid substitution at a position corresponding to position 199, position 200, position 201, and/or position 202 of SEQ ID NO: 93.

158. The contiguous polypeptide of any one of the claims 149 to 157, wherein the variant IgG Fc polypeptide comprises:
a) an amino acid substitution at position 21, position 23, position 25, position 80, position 205, and/or position 207 of SEQ ID NO: 53;
b) an amino acid substitution at position 21, position 23, and/or position 24 of SEQ ID NO: 56;
c) an amino acid substitution at position 21, position 23, position 25, position 80, and/or position 207 of SEQ ID NO: 58;
d) an amino acid substitution at position 15 and/or position 203 of SEQ ID NO: 88;
e) an amino acid substitution at position 199 and/or position 200 of SEQ ID NO: 92; and/or
f) an amino acid substitution at position 199, position 200, position 201, and/or position 202 of SEQ ID NO: 93.

159. The contiguous polypeptide of any one of the claims 149 to 158, wherein the variant IgG Fc polypeptide comprises:
a) a threonine at a position corresponding to position 21, a leucine at a position corresponding to position 23, an alanine at a position corresponding to position 25, a glycine at a position corresponding to position 80, an alanine at a position corresponding to position 205, and/or a histidine at a position corresponding to position 207 of SEQ ID NO: 53;
b) a threonine at a position corresponding to position 21, a leucine at a position corresponding to position 23, and/or an isoleucine at a position corresponding to position 24 of SEQ ID NO: 56;
c) a threonine at a position corresponding to position 21, a leucine at a position corresponding to position 23, an alanine at a position corresponding to position 25, a glycine at a position corresponding to position 80, and/or a histidine at a position corresponding to position 207 of SEQ ID NO: 58;
d) a threonine or a valine at a position corresponding to position 15 and/or a tyrosine or a valine at a position corresponding to position 203 of SEQ ID NO: 88;
e) a leucine at a position corresponding to position 199 and/or a histidine at a position corresponding to position 200 of SEQ ID NO: 92; and/or
f) a leucine at a position corresponding to position 199, a histidine at a position corresponding to position 200, an asparagine at a position corresponding to position 201, and/or a histidine at a position corresponding to position 202 of SEQ ID NO: 93.

160. The contiguous polypeptide of any one of the claims 149 to 159, wherein the variant IgG Fc polypeptide comprises:
a) a threonine at position 21, a leucine at position 23, an alanine at position 25, a glycine at position 80, an alanine at position 205, and/or a histidine at position 207 of SEQ ID NO: 53;
b) a threonine at position 21, a leucine at position 23, and/or an isoleucine at position 24 of SEQ ID NO: 56;
c) a threonine at a position 21, a leucine at position 23, an alanine at position 25, a glycine at position 80, and/or a histidine at position 207 of SEQ ID NO: 58;
d) a threonine or a valine at position 15, and/or a tyrosine or a valine at position 203 of SEQ ID NO: 88;
e) a leucine at position 199 and/or a histidine at position 200 of SEQ ID NO: 92; and/or
f) a leucine at position 199, a histidine at position 200, an asparagine at position 201, and/or a histidine at position 202 of SEQ ID NO: 93.

161. The contiguous polypeptide of any one of the claims 149 to 160, wherein the variant IgG Fc polypeptide comprises:
a) an amino acid substitution at a position corresponding to position 93 of SEQ ID NO: 54 or SEQ ID NO: 56;
b) an amino acid substitution at a position corresponding to position 87 of SEQ ID NO: 87, SEQ ID NO: 90, SEQ ID NO: 91, or SEQ ID NO: 94; or
c) an amino acid substitution at a position corresponding to position 198 of SEQ ID NO: 103, SEQ ID NO: 104, SEQ ID NO: 105, or SEQ ID NO: 106.

162. The contiguous polypeptide of any one of the claims 149 to 161, wherein the variant IgG Fc polypeptide comprises:
a) an amino acid substitution at position 93 of SEQ ID NO: 54 or SEQ ID NO: 56;
b) an amino acid substitution at position 87 of SEQ ID NO: 87, SEQ ID NO: 90, SEQ ID NO: 91, or SEQ ID NO: 94; or
c) an amino acid substitution at position 198 of SEQ ID NO: 103, SEQ ID NO: 104, SEQ ID NO: 105, or SEQ ID NO: 106.

163. The contiguous polypeptide of any one of the claims 149 to 162, wherein the variant IgG Fc polypeptide comprises:
a) an arginine at a position corresponding to position 93 of SEQ ID NO: 54 or SEQ ID NO: 56;
b) a serine at a position corresponding to position 87 of SEQ ID NO: 87, SEQ ID NO: 90, SEQ ID NO: 91, or SEQ ID NO: 94; or
c) an alanine at a position corresponding to position 198 of SEQ ID NO: 103, SEQ ID NO: 104, SEQ ID NO: 105, or SEQ ID NO: 106.

164. The contiguous polypeptide of any one of the claims 149 to 163, wherein the variant IgG Fc polypeptide comprises:
a) an arginine at position 93 of SEQ ID NO: 54 or SEQ ID NO: 56;
b) a serine at position 87 of SEQ ID NO: 87, SEQ ID NO: 90, SEQ ID NO: 91, or SEQ ID NO: 94; or
c) an alanine at position 198 of SEQ ID NO: 103, SEQ ID NO: 104, SEQ ID NO: 105, or SEQ ID NO: 106.

165. The contiguous polypeptide of any one of the claims 149 to 164, wherein the variant IgG Fc polypeptide comprises:
a) an amino acid substitution at a position corresponding to position 5, position 38, position 39, position 97, and/or position 98 of SEQ ID NO: 54; or
b) an amino acid substitution at a position corresponding to position 5, position 38, position 39, position 97, and/or position 98 of SEQ ID NO: 56.

166. The contiguous polypeptide of any one of the claims 149 to 165, wherein the variant IgG Fc polypeptide comprises:
a) an amino acid substitution at position 5, position 38, position 39, position 97, and/or position 98 of SEQ ID NO: 54; or
b) an amino acid substitution at position 5, position 38, position 39, position 97, and/or position 98 of SEQ ID NO: 56.

167. The contiguous polypeptide of any one of the claims 149 to 166, wherein the variant IgG Fc polypeptide comprises:
a) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 54; or
b) a proline at a position corresponding to position 5, a glycine at a position corresponding to position 38, an arginine at a position corresponding to position 39, an isoleucine at a position corresponding to position 97, and/or a glycine at a position corresponding to position 98 of SEQ ID NO: 56.

168. The contiguous polypeptide of any one of the claims 149 to 167, wherein the variant IgG Fc polypeptide comprises:
a) a proline at position 5, a glycine at position 38, an arginine at position 39, an isoleucine at position 97, and/or a glycine at position 98 of SEQ ID NO: 54; or
b) a proline at position 5, a glycine at position 38, an arginine at position 39, an isoleucine at position 97, and/or a glycine at position 98 of SEQ ID NO: 56.

169. The contiguous polypeptide of any one of claims 149 to 168, wherein the variant IgG Fc polypeptide comprises the amino acid sequence of SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, or SEQ ID NO: 111.

170. An isolated nucleic acid encoding the polypeptide of any one of claims 147 to 169.

171. A host cell comprising the nucleic acid of claim 170.

172. A method of producing a polypeptide comprising culturing the host cell of claim 171 and isolating the polypeptide.

173. A pharmaceutical composition comprising the polypeptide of any one of claims 147 to 169 and a pharmaceutically acceptable carrier.

174. A method of treating a companion animal having polycythemia, the method comprising administering to the subject a therapeutically effective amount of the polypeptide of any one of any one of claims 147 to 169, the nucleic acid of claim 170, or the pharmaceutical composition of claim 173.

175. The method of claim 174, wherein the polypeptide, nucleic acid, or pharmaceutical composition is administered parenterally.

176. The method of claim 174 or claim 175, wherein the polypeptide, nucleic acid, or pharmaceutical composition is administered by an intramuscular route, an intraperitoneal route, an intracerebrospinal route, a subcutaneous route, an intra-arterial route, an intrasynovial route, an intrathecal route, or an inhalation route.

177. The method of any one of claims 174 to 176, wherein the companion animal species is feline, canine, or equine.

178. The method of any one of claims 174 to 177, wherein the polycythemia is caused by a mutation in JAK2, overproduction and/or secretion of EPO from a tumor.
